(19) United States
(12) Patent Application Publication
(10) Pub. No.: US 2012/0202799 A1
(43) Pub. Date: Aug. 9, 2012

(54) Condensed Azepine Derivatives As Bromodomain Inhibitors

(76) Inventors: Miriam Crowe, Stevenage (GB); Alain Claude-Marie Daugan, Les Ulis (FR); Romain Luc Marie Gosmini, Les Ulis (FR); Richard Martin Grimes, Research Triangle Park, NC (US); Olivier Mirguet, Les Ulis (FR); Jacqueline Elizabeth Mordaunt, Stevenage (GB)

(21) Appl. No.: 13/501,557

(22) PCT Filed: Nov. 3, 2010
(86) PCT No.: PCT/EP2010/066696

§ 371 Date: Apr. 12, 2012

(30) Foreign Application Priority Data
Nov. 5, 2009 (GB) 0919426.7

(51) Int. Cl. A61K 31/5517; A61P 29/00; A61P 37/06; C07D 491/147; C12N 5/02; C07D 495/14; A61K 31/551; C07D 487/04; A61P 35/00

(52) U.S. Cl. 514/220; 540/566; 435/375; 540/560; 540/555; 514/219

(57) ABSTRACT

Benzodiazepine compounds of formula (I)

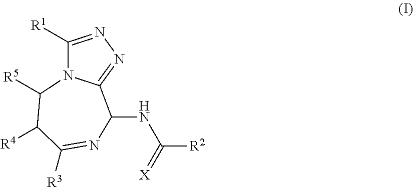

and salts thereof, pharmaceutical compositions containing such compounds and their use in therapy.

### FIELD OF THE INVENTION

The present invention relates to benzodiazepine compounds, pharmaceutical compositions containing such compounds and to their use in therapy.

### BACKGROUND OF THE INVENTION

The genomes of eukaryotic organisms are highly organised within the nucleus of the cell. The long strands of duplex DNA are wrapped around an octomer of histone proteins (most usually comprising two copies of histones H2A, H2B H3 and H4) to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. Chromatin structure plays a critical role in regulating gene transcription, which cannot occur efficiently from highly condensed chromatin. The chromatin structure is controlled by a series of post translational modifications to histone proteins, notably histones H3 and H4, and most commonly within the histone tails which extend beyond the core nucleosome structure. These modifications include acetylation, methylation, phosphorylation, ubiquitinylation, SUMOylation. These epigenetic marks are written and erased by specific enzymes, which place the tags on specific residues within the histone tail, thereby forming an epigenetic code, which is then interpreted by the cell to allow gene specific regulation of chromatin structure and thereby transcription.

Histone acetylation is most usually associated with the activation of gene transcription, as the modification loosens the interaction of the DNA and the histone octomer by changing the electrostatics. In addition to this physical change, specific proteins bind to acetylated lysine residues within histones to read the epigenetic code. Bromodomains are small (˜110 amino acid) distinct domains within proteins that bind to acetylated lysine resides commonly but not exclusively in the context of histones. There is a family of around 50 proteins known to contain bromodomains, and they have a range of functions within the cell.

The BET family of bromodomain containing proteins comprises 4 proteins (BRD2, BRD3, BRD4 and BRD-t) which contain tandem bromodomains capable of binding to two acetylated lysine residues in close proximity, increasing the specificity of the interaction. BRD2 and BRD3 are reported to associate with histones along actively transcribed genes and may be involved in facilitating transcriptional elongation (Leroy et al, Mol. Cell. 2008 30(1):51-60), while BRD4 appears to be involved in the recruitment of the pTEF-β complex to inducible genes, resulting in phosphorylation of RNA polymerase and increased transcriptional output (Hargreaves et al, Cell, 2009 138(1): 129-145). It has also been reported that BRD4 or BRD3 may fuse with NUT (nuclear protein in testis) forming novel fusion oncogenes, BRD4-NUT or BRD3-NUT, in a highly malignant form of epithelial neoplasia (French et al. Cancer Research, 2003, 63, 304-307 and French et al. Journal of Clinical Oncology, 2004, 22 (20), 4135-4139). Data suggests that BRD-NUT fusion proteins contribute to carcinogenesis (Oncogene, 2008, 27, 2237-2242). BRD-t is uniquely expressed in the testes and ovary. All family members have been reported to have some function in controlling or executing aspects of the cell cycle, and have been shown to remain in complex with chromosomes during cell division—suggesting a role in the maintenance of epigenetic memory. In addition some viruses make use of these proteins to tether their genomes to the host cell chromatin, as part of the process of viral replication (You et al Cell, 2004 117(3):349-60).

Japanese patent application JP2008-156311 discloses a benzimidazole derivative which is said to be a BRD2 bromodomain binding agent which has utility with respect to virus infection/proliferation.

Patent application WO2009/084693A1 discloses a series of thienotriazolodiazepiene derivatives that are said to inhibit the binding between an acetylated histone and a bromodomain containing protein which are said to be useful as anti-cancer agents.

A novel class of compounds have been found which inhibit the binding of bromodomains with its cognate acetylated proteins, more particularly a class of compounds that inhibit the binding of BET family bromodomains to acetylated lysine residues. Such compounds will hereafter be referred to as “bromodomain inhibitors”.

### SUMMARY OF THE INVENTION

In a first aspect of the present invention, there is provided a compound of formula (I) or a salt thereof, more particularly a compound of formula (I) or a pharmaceutically acceptable salt thereof.

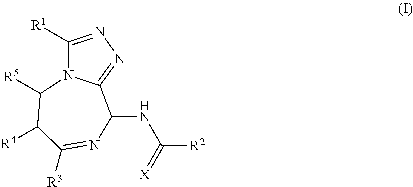

In a second aspect of the present invention, there is provided a pharmaceutical composition comprising a compound of formula (I) or a pharmaceutically acceptable salt thereof and one or more pharmaceutically acceptable carriers, diluents or excipients.

In a third aspect of the present invention, there is provided a compound of formula (I), or a pharmaceutically acceptable salt thereof for use in therapy, in particular in the treatment of diseases or conditions for which a bromodomain inhibitor is indicated.

In a fourth aspect of the present invention, there is provided a method of treating diseases or conditions for which a bromodomain inhibitor is indicated in a subject in need thereof which comprises administering a therapeutically effective amount of compound of formula (I) or a pharmaceutically acceptable salt thereof.

In a fifth aspect of the present invention, there is provided the use of a compound of formula (I), or a pharmaceutically acceptable salt thereof in the manufacture of a medicament for the treatment of diseases or conditions for which a bromodomain inhibitor is indicated.

### DETAILED DESCRIPTION OF THE INVENTION

In one embodiment, the present invention relates to compounds of formula (I) or a salt thereof.

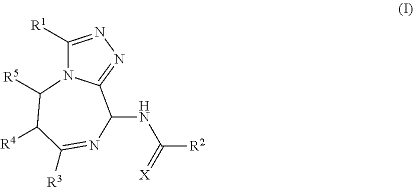

where
X is O or S;
   R¹ is C₁₋₆alkyl, haloC₁₋₆alkyl, —(CH₂)ₙOR^{1a} or —(CH₂)ₘNR^{1b}R^{1c}; wherein R^{1a} is hydrogen, C₁₋₆alkyl or haloC₁₋₆alkyl; R^{1b} and R^{1c}, which may be the same or different, are hydrogen, C₁₋₆alkyl or haloC₁₋₆alkyl; and m and n, which may be the same or different, are 1, 2 or 3;
R² is R^{2a}, —OR^{2b} or —NR^{2c}R^{2d}; wherein R^{2a} and R^{2b} are carbocyclyl, carbocyclylC₁₋₄alkyl, heterocyclyl or heterocyclylC₁₋₄alkyl, or R^{2a} is carbocyclylethenyl or heterocyclylethenyl, wherein any of the carbocyclyl or heterocyclyl groups defined for R^{2a} or R^{2b} are optionally substituted by one or more groups independently selected from halogen, C₁₋₆alkyl, haloC₁₋₆alkyl, C₁₋₆alkoxy, haloC₁₋₆alkoxy, nitro, cyano, dimethylamino, benzoyl and azido; or two adjacent groups on any of the carbocyclyl or heterocyclyl groups defined for R^{2a} or R^{2b} together with the interconnecting atoms form a 5 or 6-membered ring which ring may contain 1 or 2 heteroatoms independently selected from O, S and N; or R^{2a} and R^{2b} are C₁₋₆alkyl or haloC₁₋₆alkyl; and R^{2c} and R^{2d}, which may be the same or different, are carbocyclyl, carbocyclylC₁₋₄alkyl, heterocyclyl or heterocyclylC₁₋₄alkyl, wherein any of the carbocyclyl or heterocyclyl groups defined for R^{2c} and R^{2d} are optionally substituted by one or more groups independently selected from: halogen, C₁₋₆alkyl, haloC₁₋₆alkyl, C₁₋₆alkoxy, haloC₁₋₆alkoxy, nitro, cyano and —CO₂C₁₋₄alkyl; or two adjacent groups on any of the carbocyclyl or heterocyclyl groups defined for R^{2c} and R^{2d} together with the interconnecting atoms form a 5 or 6-membered ring which ring may contain 1 or 2 heteroatoms independently selected from: O, S and N; or R^{2c} and R^{2d} are hydrogen, C₁₋₆alkyl or haloC₁₋₆alkyl;
R³ is carbocyclyl or heterocyclyl, either of which is optionally substituted independently by one or more halogen, C₁₋₆alkyl, haloC₁₋₆alkyl, C₁₋₆alkoxy, haloC₁₋₆alkoxy, nitro or cyano; or R³ is C₁₋₆alkyl; and
R⁴ and R⁵ together with the interconnection carbon atoms form a benzene or aromatic heterocyclic ring, each of which is optionally substituted.

In another embodiment, the present invention relates to compounds of formula (I) or a salt thereof

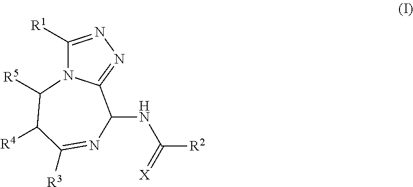

where
X is O or S;
R¹ is C₁₋₆alkyl, haloC₁₋₆alkyl, —(CH₂)ₙOR^{1a} or —(CH₂)ₘNR^{1b}R^{1c}; wherein R^{1a} is hydrogen, C₁₋₆alkyl or haloC₁₋₆alkyl; R^{1b} and R^{1c}, which may be the same or different, are hydrogen, C₁₋₆alkyl or haloC₁₋₆alkyl; and m and n, which may be the same or different, are 1, 2 or 3;
R² is R^{2a}, —OR^{2b} or —NR^{2c}R^{2d}; wherein R^{2a} and R^{2b} are carbocyclyl, carbocyclylC₁₋₄alkyl, heterocyclyl or heterocyclylC₁₋₄alkyl, or R^{2a} is carbocyclylethenyl or heterocyclylethenyl, wherein any of the carbocyclyl or heterocyclyl groups defined for R^{2a} or R^{2b} are optionally substituted by one or more groups independently selected from halogen, C₁₋₆alkyl, haloC₁₋₆alkyl, C₁₋₆alkoxy, haloC₁₋₆alkoxy, nitro, cyano, dimethylamino, benzoyl and azido; or two adjacent groups on any of the carbocyclyl or heterocyclyl groups defined for R^{2a} or R^{2b} together with the interconnecting atoms form a 5 or 6-membered ring which ring may contain 1 or 2 heteroatoms independently selected from O, S and N; or R^{2a} and R^{2b} are C₁₋₆alkyl or haloC₁₋₆alkyl; and R^{2c} and R^{2d}, which may be the same or different, are carbocyclyl, carbocyclylC₁₋₄alkyl, heterocyclyl or heterocyclylC₁₋₄alkyl, wherein any of the carbocyclyl or heterocyclyl groups defined for R^{2c} and R^{2d} are optionally substituted by one or more groups independently selected from: halogen, C₁₋₆alkyl, haloC₁₋₆alkyl, C₁₋₆alkoxy, haloC₁₋₆alkoxy, nitro and cyano; or two adjacent groups on any of the carbocyclyl or heterocyclyl groups defined for R^{2c} and R^{2d} together with the interconnecting atoms form a 5 or 6-membered ring which ring may contain 1 or 2 heteroatoms independently selected from: O, S and N; or R^{2c} and R^{2d} are hydrogen, C₁₋₆alkyl or haloC₁₋₆alkyl;
R³ is carbocyclyl or heterocyclyl, either of which is optionally substituted independently by one or more halogen, C₁₋₆alkyl, haloC₁₋₆alkyl, C₁₋₆alkoxy, haloC₁₋₆alkoxy, nitro or cyano; or R³ is C₁₋₆alkyl; and
R⁴ and R⁵ together with the interconnection carbon atoms form a benzene or aromatic heterocyclic ring, each of which is optionally substituted.

In one embodiment of the invention the compound of formula (I) is the S-enantiomer.

Unless otherwise indicated, any alkyl group may be straight or branched and is of 1 to 6 carbon atoms, preferably 1 to 4 and particularly 1 to 3 carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

Unless otherwise indicated, any carbocyclyl group contains 3 to 14 ring-atoms for example, 3 to 10 ring-atoms, or in a further example, 3 to 8 ring-atoms and may be saturated, unsaturated or aromatic. Preferred saturated carbocyclyl groups are cyclopropyl, cyclopentyl or cyclohexyl. Preferred unsaturated carbocyclyl groups contain up to 3 double bonds. A preferred aromatic carbocyclyl group is phenyl. The term carbocylic should be similarly construed. In addition, the term carbocyclyl includes any fused combination of carbocyclyl groups, for example naphthyl, phenanthryl, indanyl and indenyl.

Unless otherwise indicated, any heterocyclyl group contains 5 to 9 ring-atoms for example, 5 to 7 ring-atoms, up to 4 of which may be hetero-atoms such as nitrogen, oxygen and sulfur, and may be saturated, unsaturated or aromatic. Examples of heterocyclyl groups are furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl and thiazolinyl. In addition, the term heterocyclyl includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl. The term heterocyclic should be similarly construed.

Halo is fluoro, chloro, bromo or iodo.

In one embodiment the invention provides a compound of formula (I) wherein R² is —OR^{2b}. In one embodiment, R^{2b} is C₁₋₆alkyl, benzyl or phenylC₁₋₆alkyl wherein benzyl is optionally substituted by fluoro. In another embodiment, R^{2b} is ethyl, isopropyl, benzyl, 4-fluorobenzyl or —CH(CH₃)phenyl.

In one embodiment the invention provides a compound of formula (I) or a salt thereof wherein R² is —OR^{2b}, with the proviso that the compound of formula (I) is not:
a) phenylmethyl [6-(4-chlorophenyl)-1-methyl-4H-[1,2,4]triazolo[4,3-a][1,4]benzodiazepin-4-yl]carbamate;
b) phenyl [4-(2-chlorophenyl)-2-ethyl-9-methyl-6H-thieno[3,2-f][1,2,4]triazolo[4,3-a][1,4]diazepin-6-yl]carbamate;
c) phenylmethyl [6-phenyl-1-methyl-4H-[1,2,4]triazolo[4,3-a][1,4]benzodiazepin-4-yl]carbamate.

Preferably X is O.

In one embodiment there is provided a compound or a salt thereof in which
X is O;
R¹ is C₁₋₆alkyl, haloC₁₋₆alkyl, —(CH₂)ₙOR^{1a} or —(CH₂)ₘNR^{1b}R^{1c}; wherein R^{1a} is hydrogen, C₁₋₆alkyl or haloC₁₋₆alkyl; R^{1b} and R^{1c}, which may be the same or different, are hydrogen, C₁₋₆alkyl or haloC₁₋₆alkyl; and m and n, which may be the same or different, are 1, 2 or 3;
R² is R^{2a}, —OR^{2b} or —NR^{2c}R^{2d}; wherein R^{2a} and R^{2b} are carbocyclyl, carbocyclylC₁₋₄alkyl, heterocyclyl or heterocyclylC₁₋₄alkyl, wherein any of the carbocyclyl or heterocyclyl groups are optionally substituted by halogen, C₁₋₆alkyl, haloC₁₋₆alkyl, C₁₋₆alkoxy, haloC₁₋₆alkoxy, nitro or cyano; or two adjacent groups on any of the carbocyclyl or heterocyclyl groups together with the interconnecting atoms form a 5 or 6-membered ring which ring may contain 1 or 2 heteroatoms independently selected from O, S or N; or R^{2a} and R^{2b} are C₁₋₆alkyl or haloC₁₋₆alkyl; and R^{2c} and R^{2d}, which may be the same or different, are carbocyclyl, carbocyclylC₁₋₄alkyl, heterocyclyl or heterocyclylC₁₋₄alkyl, wherein any of the carbocyclyl or heterocyclyl groups are optionally substituted by halogen, C₁₋₆alkyl, haloC₁₋₆alkyl, C₁₋₆alkoxy, haloC₁₋₆alkoxy, nitro or cyano; or two adjacent groups on any of the carbocyclyl or heterocyclyl groups together with the interconnecting atoms form a 5 or 6-membered ring which ring may contain 1 or 2 heteroatoms independently selected from O, S or N; or R^{2c} and R^{2d} are hydrogen, C₁₋₆alkyl or haloC₁₋₆alkyl;
R³ is carbocyclyl or heterocyclyl, either of which is optionally substituted by halogen, C₁₋₆alkyl, haloC₁₋₆alkyl, C₁₋₆alkoxy, haloC₁₋₆alkoxy, nitro or cyano; or R³ is C₁₋₆alkyl; and
R⁴ and R⁵ together with the interconnection carbon atoms form a benzene or aromatic heterocyclic ring, each of which is optionally substituted.

In one embodiment R¹ is C₁₋₆alkyl. In a particular embodiment R¹ is methyl.

In one embodiment R² is R^{2a}, —OR^{2b} or —NR^{2c}R^{2d}; wherein
R^{2a} and R^{2b} are phenyl, benzyl or C₃₋₆cycloalkyl, any ring of which is optionally substituted by one or more groups independently selected from halogen, C₁₋₆alkyl, haloC₁₋₆alkyl, C₁₋₆alkoxy, haloC₁₋₆alkoxy, nitro and cyano; or two adjacent groups on any of the rings together with the interconnecting atoms form a methylenedioxy group; or R^{2a} and R^{2b} are C₁₋₆alkyl or haloC₁₋₆alkyl; and
R^{2d} and R^{2d}, which may be the same or different, are phenyl, benzyl or C₁₋₆cycloalkyl, any ring of which is optionally substituted by one or more groups independently selected from halogen, C₁₋₆alkyl, haloC₁₋₆alkyl, C₁₋₆alkoxy, haloC₁₋₆alkoxy, nitro and cyano; or two adjacent groups on any of the rings together with the interconnecting atoms form a methylenedioxy group; or R^{2d} and R^{2d} are hydrogen, C₁₋₆alkyl or haloC₁₋₆alkyl.

In a particular embodiment R² is —OR^{2b}. R^{2b} is preferably C₁₋₆alkyl or benzyl.

In one embodiment, there is provided a compound of formula (I) or a salt thereof wherein R² is R^{2a}. In one embodiment R^{2a} is carbocycylethenyl optionally substituted by one or more groups independently selected from halogen, C₁₋₆alkyl, haloC₁₋₆alkyl, C₁₋₆alkoxy, haloC₁₋₆alkoxy, nitro, cyano, dimethylamino, benzoyl and azido. In another embodiment, R^{2a} is carbocycylethenyl optionally substituted by one group selected from fluoro, chloro and methoxy. In another embodiment, R^{2a} is carbocycyl or heterocyclyl optionally substituted by one or more groups independently selected from C₁₋₆alkyl, C₁₋₆alkoxy and benzoyl. In a further embodiment, R^{2a} is phenyl, napthylenyl or indolyl optionally substituted by one group selected from methyl, methoxy and benzoyl.

In one embodiment, there is provided a compound of formula (I) or a salt thereof wherein R² is —NR^{2d}R^{2d}. In one embodiment, R^{2d} is hydrogen and R^{2d} is phenyl or benzyl optionally substituted by one group selected from halogen, C₁₋₆alkyl, C₁₋₆alkoxy and —CO₂C₁₋₄alkyl. In another embodiment, R^{2d} is hydrogen and R^{2d} is substituted by one group selected from bromine, ethyl, methoxy and —CO₂CH₂CH₃.

In one embodiment R³ is phenyl, thienyl, furyl or pyridyl, any of which are optionally substituted by one or more groups independently selected from halogen, C₁₋₆alkyl, haloC₁₋₆alkyl, C₁₋₆alkoxy, haloC₁₋₆alkoxy, nitro and cyano; or R³ is C₁₋₆alkyl. In another embodiment, R³ is phenyl optionally substituted by one or more groups independently selected from halogen, C₁₋₆alkyl, haloC₁₋₆alkyl, C₁₋₆alkoxy, haloC₁₋₆alkoxy, nitro and cyano. In another embodiment, R³ is phenyl optionally substituted by one group selected from methyl, chloro and methoxy. In another embodiment, R³ is phenyl substituted at the para position by one or more groups independently selected from halogen, C₁₋₆alkyl, haloC₁₋₆alkyl, C₁₋₆alkoxy, haloC₁₋₆alkoxy, nitro and cyano. In a further embodiment R³ is unsubstituted phenyl.

In one embodiment R⁴ and R⁵, together with the interconnecting atoms, form a benzene, a thiophene, a furan or a benzofuran ring (more preferably a benzene, a thiophene or a furan ring), any of which are optionally substituted by one or more groups independently selected from halogen, C₁₋₆alkyl, C₂₋₆alkenyl, haloC₁₋₆alkyl, C₁₋₆alkoxy, haloC₁₋₆alkoxy, nitro, cyano and heterocyclyl. A preferred heterocyclyl group is furyl or thienyl. In one embodiment, R⁴ and R⁵ together with the interconnecting atoms form an optionally substituted benzene ring. In another embodiment, R⁴ and R⁵ together with the interconnecting atoms form a benzene ring, which is optionally substituted by iodine.

In a particular embodiment
X is O;
R¹ is C₁₋₆alkyl;
R² is R^{2a}, —OR^{2b} or —NR^{2d}R^{2d}; wherein R^{2a} and R^{2b} are phenyl, benzyl or C₁₋₆cycloalkyl, any ring of which is optionally substituted by one or more groups independently selected from halogen, C₁₋₆alkyl, haloC₁₋₆alkyl, C₁₋₆alkoxy, haloC₁₋₆alkoxy, nitro and cyano; or two adjacent groups on any of the rings together with the interconnecting atoms form a methylenedioxy group; or R^{2a} and R^{2b} are C₁₋₆alkyl or haloC₁₋₆alkyl; and R^{2d} and R^{2d}, which may be the same or different, are phenyl, benzyl or C₃₋₆cycloalkyl, any ring of which is optionally substituted by one or more groups independently selected from halogen, C₁₋₆alkyl, haloC₁₋₆alkyl, C₁₋₆alkoxy, haloC₁₋₆alkoxy, nitro and cyano; or two adjacent groups on any of the rings together with the interconnecting atoms form a methylenedioxy group; or R^{2d} and R^{2d} are hydrogen, C₁₋₆alkyl or haloC₁₋₆alkyl;
R³ is phenyl, thienyl, furyl or pyridyl, any of which are optionally substituted by one or more groups independently selected from halogen, C₁₋₆alkyl, haloC₁₋₆alkyl, C₁₋₆alkoxy, haloC₁₋₆alkoxy, nitro and cyano; or R³ is C₁₋₆alkyl; and
R⁴ and R⁵, together with the interconnecting atoms, form a benzene, a thiophene or a furan ring, any of which are optionally substituted by one or more groups independently selected from halogen, C₁₋₆alkyl, C₂₋₆alkenyl, haloC₁₋₆alkyl, C₁₋₆alkoxy, haloC₁₋₆alkoxy, nitro, cyano and heterocyclyl.

While the embodiments for each variable have generally been listed above separately for each variable this invention includes those compounds in which several or each embodiment in formula (I) is selected from each of the embodiments listed above. Therefore, this invention is intended to include all combinations of embodiments for each variable described hereinabove including salts thereof.

In one embodiment the compound of formula (I) is selected from:
phenylmethyl [6-(4-fluorophenyl)-1-methyl-4H-[1,2,4]triazolo[4,3-a][1,4]benzodiazepin-4-yl]carbamate (Example 1);
phenylmethyl [6-(4-chlorophenyl)-1-ethyl-4H-[1,2,4]triazolo[4,3-a][1,4]benzodiazepin-4-yl]carbamate (Example 3);
ethyl [6-(4-chlorophenyl)-1-methyl-4H-[1,2,4]triazolo[4,3-a][1,4]benzodiazepin-4-yl]carbamate (Example 4);
ethyl [6-(4-chlorophenyl)-1-ethyl-4H-[1,2,4]triazolo[4,3-a][1,4]benzodiazepin-4-yl]carbamate (Example 5);
phenylmethyl [1-methyl-8-(methyloxy)-6-phenyl-4H-[1,2,4]triazolo[4,3-a][1,4]benzodiazepin-4-yl]carbamate (Example 6);
phenylmethyl {1-methyl-6-[4-(methyloxy)phenyl]-4H-[1,2,4]triazolo[4,3-a][1,4]benzodiazepin-4-yl}carbamate (Example 7);
phenylmethyl [1-methyl-6-(4-methylphenyl)-4H-[1,2,4]triazolo[4,3-a][1,4]benzodiazepin-4-yl]carbamate (Example 8);
phenylmethyl {1-methyl-6-[3-(methyloxy)phenyl]-4H-[1,2,4]triazolo[4,3-a][1,4]benzodiazepin-4-yl}carbamate (Example 9);
phenylmethyl (1,9-dimethyl-6-phenyl-4H-[1,2,4]triazolo[4,3-a][1,4]benzodiazepin-4-yl)carbamate (Example 10);
phenylmethyl (8-chloro-1-methyl-6-phenyl-4H-[1,2,4]triazolo[4,3-a][1,4]benzodiazepin-4-yl)carbamate (Example 11);
phenylmethyl (9-methyl-4-phenyl-6H-thieno[3,2-f][1,2,4]triazolo[4,3-a][1,4]diazepin-6-yl)carbamate (Example 12);
phenylmethyl (1-methyl-6-phenyl-4H-thieno[2,3-f][1,2,4]triazolo[4,3-a][1,4]diazepin-4-yl)carbamate (Example 13);
phenylmethyl [6-(4-chlorophenyl)-1-methyl-8-(methyloxy)-4H-[1,2,4]triazolo[4,3-a][1,4]benzodiazepin-4-yl]carbamate (Example 14);
phenylmethyl (1-ethyl-6-phenyl-4H-[1,2,4]triazolo[4,3-a][1,4]benzodiazepin-4-yl)carbamate (Example 15);
phenylmethyl (1-methyl-6-phenyl-4H-[1]benzofuro[2,3-f][1,2,4]triazolo[4,3-a][1,4]diazepin-4-yl)carbamate (Example 18);
phenylmethyl [6-(4-chlorophenyl)-1-methyl-4H-[1]-benzofuro[2,3-f][1,2,4]triazolo[4,3-a][1,4]diazepin-4-yl]carbamate (Example 20);
phenylmethyl {1-ethyl-6-[4-(methyloxy)phenyl]-4H-[1,2,4]triazolo[4,3-a][1,4]benzodiazepin-4-yl}carbamate (Example 21);
phenylmethyl [1-ethyl-6-(4-fluorophenyl)-4H-[1,2,4]triazolo[4,3-a][1,4]benzodiazepin-4-yl]carbamate (Example 22);
ethyl [6-(4-fluorophenyl)-1-methyl-4H-[1,2,4]triazolo[4,3-a][1,4]benzodiazepin-4-yl]carbamate (Example 23);
phenylmethyl [6-(2-fluorophenyl)-8-(2-furanyl)-1-methyl-4H-[1,2,4]triazolo[4,3-a][1,4]benzodiazepin-4-yl]carbamate (Example 26);
(+)-phenylmethyl (1-methyl-6-phenyl-4H-[1,2,4]triazolo[4,3-a][1,4]benzodiazepin-4-yl)carbamate (Example 27);
(+)-ethyl [6-(4-chlorophenyl)-1-methyl-4H-[1,2,4]triazolo[4,3-a][1,4]benzodiazepin-4-yl]carbamate (Example 28);
ethyl (1-methyl-6-phenyl-4H-[1,2,4]triazolo[4,3-a][1,4]benzodiazepin-4-yl)carbamate (Example 29);
ethyl [1-ethyl-6-(4-fluorophenyl)-4H-[1,2,4]triazolo[4,3-a][1,4]benzodiazepin-4-yl]carbamate (Example 30);
ethyl {1-methyl-6-[4-(methyloxy)phenyl]-4H-[1,2,4]triazolo[4,3-a][1,4]benzodiazepin-4-yl}carbamate 5Example 31);
(+)-ethyl 1-methyl-6-phenyl-4H-[1,2,4]triazolo[4,3-a][1,4]benzodiazepin-4-ylcarbamate (Example 32);
cyclohexyl (1-methyl-6-phenyl-4H-[1,2,4]triazolo[4,3-a][1,4]benzodiazepin-4-yl)carbamate (Example 37);
methyl (1-methyl-6-phenyl-4H-[1,2,4]triazolo[4,3-a][1,4]benzodiazepin-4-yl)carbamate (Example 39);
2,2,2-trifluoroethyl (1-methyl-6-phenyl-4H-[1,2,4]triazolo[4,3-a][1,4]benzodiazepin-4-yl)carbamate (Example 40);
2-(1H-imidazol-1-yl)ethyl (1-methyl-6-phenyl-4H-[1,2,4]triazolo[4,3-a][1,4]benzodiazepin-4-yl)carbamate (Example 41);
2-(4-methyl-1,3-thiazol-5-yl)ethyl(1-methyl-6-phenyl-4H-[1,2,4]triazolo[4,3-a][1,4]benzodiazepin-4-yl)carbamate (Example 42);
2-thienylmethyl (1-methyl-6-phenyl-4H-[1,2,4]triazolo[4,3-a][1,4]benzodiazepin-4-yl)carbamate (Example 43);
2-furanylmethyl (1-methyl-6-phenyl-4H-[1,2,4]triazolo[4,3-a][1,4]benzodiazepin-4-yl)carbamate (Example 44);
[4-(methyloxy)phenyl]methyl (1-methyl-6-phenyl-4H-[1,2,4]triazolo[4,3-a][1,4]benzodiazepin-4-yl)carbamate (Example 45);
2-pyridinylmethyl (1-methyl-6-phenyl-4H-[1,2,4]triazolo[4,3-a][1,4]benzodiazepin-4-yl)carbamate (Example 46);
(4-chlorophenyl)methyl (1-methyl-6-phenyl-4H-[1,2,4]triazolo[4,3-a][1,4]benzodiazepin-4-yl)carbamate (Example 47);
cyclopentylmethyl (1-methyl-6-phenyl-4H-[1,2,4]triazolo[4,3-a][1,4]benzodiazepin-4-yl)carbamate (Example 48);
cyclopentyl (1-methyl-6-phenyl-4H-[1,2,4]triazolo[4,3-a][1,4]benzodiazepin-4-yl)carbamate (Example 49);
2-cyclopropylethyl (1-methyl-6-phenyl-4H-[1,2,4]triazolo[4,3-a][1,4]benzodiazepin-4-yl)carbamate (Example 50); and
cyclobutylmethyl (1-methyl-6-phenyl-4H-[1,2,4]triazolo[4,3-a][1,4]benzodiazepin-4-yl)carbamate (Example 52); or a salt thereof.

In another embodiment the compound of formula (I) is selected from:
ethyl [6-(4-chlorophenyl)-1-methyl-4H-[1,2,4]triazolo[4,3-a][1,4]benzodiazepin-4-yl]carbamate (Example 4);
phenylmethyl [1-methyl-8-(methyloxy)-6-phenyl-4H-[1,2,4]triazolo[4,3-a][1,4]benzodiazepin-4-yl]carbamate (Example 6);
phenylmethyl {1-methyl-6-[4-(methyloxy)phenyl]-4H-[1,2,4]triazolo[4,3-a][1,4]benzodiazepin-4-yl}carbamate (Example 7);
phenylmethyl [1-methyl-6-(4-methylphenyl)-4H-[1,2,4]triazolo[4,3-a][1,4]benzodiazepin-4-yl]carbamate (Example 8);
phenylmethyl {1-methyl-6-[3-(methyloxy)phenyl]-4H-[1,2,4]triazolo[4,3-a][1,4]benzodiazepin-4-yl}carbamate (Example 9);
phenylmethyl (9-methyl-4-phenyl-6H-thieno[3,2-f][1,2,4]triazolo[4,3-a][1,4]diazepin-6-yl)carbamate (Example 12);
phenylmethyl (8-iodo-1-methyl-6-phenyl-4H-[1,2,4]triazolo[4,3-a][1,4]benzodiazepin-4-yl)carbamate (Example 24);
(+)-phenylmethyl (1-methyl-6-phenyl-4H-[1,2,4]triazolo[4,3-a][1,4]benzodiazepin-4-yl)carbamate (Example 27);
(+)-ethyl [6-(4-chlorophenyl)-1-methyl-4H-[1,2,4]triazolo[4,3-a][1,4]benzodiazepin-4-yl]carbamate (Example 28);
ethyl (1-methyl-6-phenyl-4H-[1,2,4]triazolo[4,3-a][1,4]benzodiazepin-4-yl)carbamate (Example 29);
ethyl {1-methyl-6-[4-(methyloxy)phenyl]-4H-[1,2,4]triazolo[4,3-a][1,4]benzodiazepin-4-yl}carbamate (Example 31);
(+)-ethyl 1-methyl-6-phenyl-4H-[1,2,4]triazolo[4,3-a][1,4]benzodiazepin-4-ylcarbamate (Example 32);
(4-fluorophenyl)methyl (1-methyl-6-phenyl-4H-[1,2,4]triazolo[4,3-a][1,4]benzodiazepin-4-yl)carbamate (Example 34);
(1S)-1-phenylethyl (1-methyl-6-phenyl-4H-[1,2,4]triazolo[4,3-a][1,4]benzodiazepin-4-yl)carbamate (Example 36);
6-(methyloxy)-N-(1-methyl-6-phenyl-4H-[1,2,4]triazolo[4,3-a][1,4]benzodiazepin-4-yl)-1H-indole-2-carboxamide (Example 53);
N-(1-methyl-6-phenyl-4H-[1,2,4]triazolo[4,3-a][1,4]benzodiazepin-4-yl)-4-(phenylcarbonyl)benzamide (Example 54);
(2E)-3-[4-(methyloxy)phenyl]-N-(1-methyl-6-phenyl-4H-[1,2,4]triazolo[4,3-a][1,4]benzodiazepin-4-yl)-2-propenamide (Example 56);
(2E)-3-(4-chlorophenyl)-N-(1-methyl-6-phenyl-4H-[1,2,4]triazolo[4,3-a][1,4]benzodiazepin-4-yl)-2-propenamide (Example 57);
(2E)-N-(1-methyl-6-phenyl-4H-[1,2,4]triazolo[4,3-a][1,4]benzodiazepin-4-yl)-3-(2-thienyl)-2-propenamide (Example 58);
5-methyl-N-(1-methyl-6-phenyl-4H-[1,2,4]triazolo[4,3-a][1,4]benzodiazepin-4-yl)-1H-indole-2-carboxamide (Example 61);
(2E)-N-(1-methyl-6-phenyl-4H-[1,2,4]triazolo[4,3-a][1,4]benzodiazepin-4-yl)-3-phenyl-2-propenamide (Example 64);
(2E)-3-(4-fluorophenyl)-N-(1-methyl-6-phenyl-4H-[1,2,4]triazolo[4,3-a][1,4]benzodiazepin-4-yl)-2-propenamide (Example 65);
N-(1-methyl-6-phenyl-4H-[1,2,4]triazolo[4,3-a][1,4]benzodiazepin-4-yl)-N′-phenyl urea (Example 70);
N-(1-methyl-6-phenyl-4H-[1,2,4]triazolo[4,3-a][1,4]benzodiazepin-4-yl)-N′-(phenylmethyl)urea (Example 71);
N-{[4-(methyloxy)phenyl]methyl}-N′-(1-methyl-6-phenyl-4H-[1,2,4]triazolo[4,3-a][1,4]benzodiazepin-4-yl)urea (Example 75);
3-bromo-N-(1-methyl-6-phenyl-4H-benzo[f][1,2,4]triazolo[4,3-a][1,4]diazepin-4-yl)benzamide (Example 85);
N-(1-methyl-6-phenyl-4H-benzo[f][1,2,4]triazolo[4,3-a][1,4]diazepin-4-yl)-2-naphthamide (Example 86);
phenylmethyl (1-methyl-6-phenyl-4H-[1,2,4]triazolo[4,3-a][1,4]benzodiazepin-4-yl)carbamate (Example 87);
ethyl 4-({[(1-methyl-6-phenyl-4H-[1,2,4]triazolo[4,3-a][1,4]benzodiazepin-4-yl)amino]carbonyl}amino)benzoate (Example 88);
1-methylethyl (1-methyl-6-phenyl-4H-[1,2,4]triazolo[4,3-a][1,4]benzodiazepin-4-yl)carbamate (Example 89); and
4-ethyl-N-(1-methyl-6-phenyl-4H-[1,2,4]triazolo[4,3-a][1,4]benzodiazepin-4-yl)benzamide (Example 97); or a salt thereof.

In a further embodiment the compound of formula (I) is selected from:
(+)-phenylmethyl (1-methyl-6-phenyl-4H-[1,2,4]triazolo[4,3-a][1,4]benzodiazepin-4-yl)carbamate (Example 27);
(+)-ethyl [6-(4-chlorophenyl)-1-methyl-4H-[1,2,4]triazolo[4,3-a][1,4]benzodiazepin-4-yl]carbamate (Example 28);
(+)-ethyl 1-methyl-6-phenyl-4H-[1,2,4]triazolo[4,3-a][1,4]benzodiazepin-4-ylcarbamate (Example 32);
6-(methyloxy)-N-(1-methyl-6-phenyl-4H-[1,2,4]triazolo[4,3-a][1,4]benzodiazepin-4-yl)-1H-indole-2-carboxamide (Example 53);
(2E)-N-(1-methyl-6-phenyl-4H-[1,2,4]triazolo[4,3-a][1,4]benzodiazepin-4-yl)-3-phenyl-2-propenamide (Example 64); and
(2E)-3-(4-fluorophenyl)-N-(1-methyl-6-phenyl-4H-[1,2,4]triazolo[4,3-a][1,4]benzodiazepin-4-yl)-2-propenamide (Example 65);
   or a salt thereof.

It will be appreciated that the present invention covers compounds of formula (I) as the free base and as salts thereof, for example as a pharmaceutically acceptable salt thereof. In one embodiment the invention relates to compounds of formula (I) and pharmaceutically acceptable salts thereof.

Because of their potential use in medicine, salts of the compounds of formula (I) are desirably pharmaceutically acceptable. Suitable pharmaceutically acceptable salts can include acid or base addition salts. As used herein, the term ‘pharmaceutically acceptable salt’ means any pharmaceutically acceptable salt or solvate of a compound of formula (I), which upon administration to the recipient is capable of providing (directly or indirectly). For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent.

A pharmaceutically acceptable base addition salt can be formed by reaction of a compound of formula (I) with a suitable inorganic or organic base, (e.g. triethylamine, ethanolamine, triethanolamine, choline, arginine, lysine or histidine), optionally in a suitable solvent, to give the base addition salt which is usually isolated, for example, by crystallisation and filtration. Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

A pharmaceutically acceptable acid addition salt can be formed by reaction of a compound of formula (I) with a suitable inorganic or organic acid (such as hydrobromic, hydrochloric, sulphuric, nitric, phosphoric, succinc, maleic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

Other non-pharmaceutically acceptable salts, e.g. formates, oxalates or trifluoroacetates, may be used, for example in the isolation of the compounds of formula (I), and are included within the scope of this invention.

The invention includes within its scope all possible stoichiometric and non-stoichiometric forms of the salts of the compounds of formula (I).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or capable of forming hydrogen bonds such as water, xylene, N-methylpyrrolidinone, methanol and ethanol may be used to form solvates. Methods for identification of solvates include, but are not limited to, NMR and microanalysis. Solvates of the compounds of formula (I) are within the scope of the invention.

The invention includes within its scope all possible stoichiometric and non-stoichiometric forms of the solvates of the compounds of formula (I).

The invention encompasses all prodrugs, of the compounds formula (I) and pharmaceutically acceptable salts thereof, which upon administration to the recipient are capable of providing (directly or indirectly) a compound of formula (I) or a pharmaceutically acceptable salt thereof, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives.

The compounds of formula (I) may be in crystalline or amorphous form. Furthermore, some of the crystalline forms of the compounds of formula (I) may exist as polymorphs, which are included within the scope of the present invention. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (SSNMR).

Certain of the compounds described herein may contain one or more chiral atoms so that optical isomers, e.g. enantiomers or diastereoisomers, may be formed. Accordingly, the present invention encompasses all isomers of the compounds of formula (I) whether as individual isomers isolated such as to be substantially free of the other isomer (i.e. pure) or as mixtures (i.e. racemates and racemic mixtures).

Similarly the invention also extends to conformational isomers of compounds of formula (I) and any geometric (cis and/or trans) isomers of said compounds.

An individual isomer isolated such as to be substantially free of the other isomer (i.e. pure) may be isolated such that less than 10%, particularly less than about 1%, for example less than about 0.1% of the other isomer is present.

Separation of isomers may be achieved by conventional techniques known to those skilled in the art, e.g. by fractional crystallisation, chromatography or HPLC.

Certain compounds of formula (I) may exist in one of several tautomeric forms. It will be understood that the present invention encompasses all tautomers of the compounds of formula (I) whether as individual tautomers or as mixtures thereof.

It will be appreciated from the foregoing that included within the scope of the invention are solvates, isomers and polymorphic forms of the compounds of formula (I) and salts thereof.

The compounds of formula (I) may be made by a variety of methods, including standard chemistry. Any previously defined variable will continue to have the previously defined meaning unless otherwise indicated. Illustrative general synthetic methods are set out below and then specific compounds of formula (I) are prepared in the working Examples. These processes form further aspects of the present invention.

Throughout the specification, general formulae are designated by Roman numerals (I), (II), (III), (IV) etc. Subsets of these general formulae are defined as (Ia), (Ib), (Ic) etc . . . (IVa), (IVb), (IVc) etc.

Compounds of formula (Ia), i.e. compounds of general formula (I) where R² is OR^{2b} and X is O, may be prepared according to reaction scheme 1 by reacting compounds of formula (III) with hydrazine hydrate followed by reaction of the resulting hydrazone (II) with R¹COCl or R¹C(OR)₃. Preferably hydrazone (II) is used without further purification and is reacted with R¹COCl at room temperature.

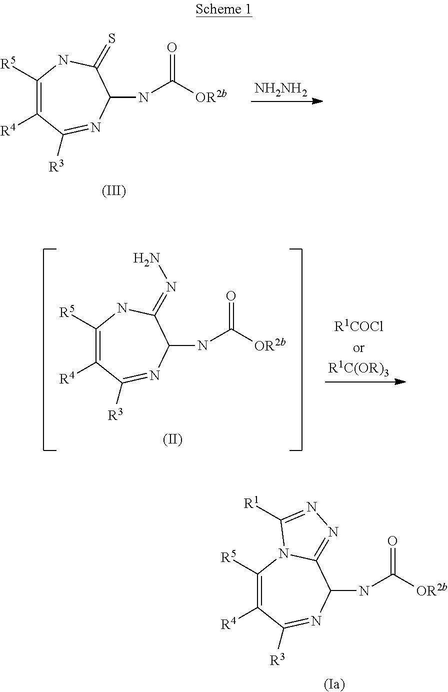

Alternatively compounds of formula (Ia), i.e. compounds of formula (I) where R² is OR^{2a} and X is O may be prepared according to reaction scheme 2, by reacting compounds of formula (IV) either with a) compounds of formula ClCOOR^{2b}, b) compounds of formula (X), c) a combination of 1,1-carbonyldiimidazole and compounds of formula R^{2b}OH, or d) compounds of formula R^{2b}OH, triphosgene and pyridine. Preferred conditions comprise reacting compounds of formula (IV) with ClCOOR^{2b} in the presence of triethylamine at room temperature. Compounds of formula (X) may be prepared by reacting 4-nitrophenylchloroformate with the R^{2b}OH in dichloromethane and pyridine.

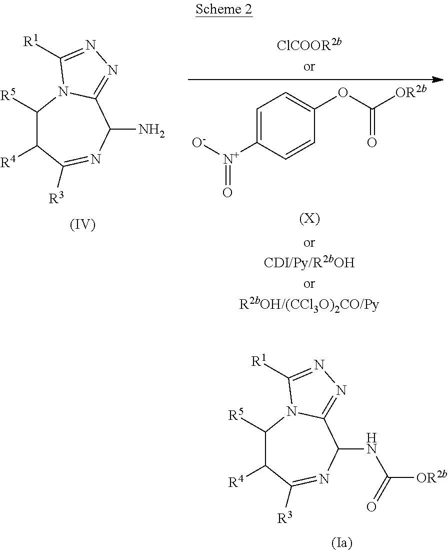

Compounds of formula (Ib), i.e. compounds of formula (I) where R² is R^{2a} and X is O may be prepared according to reaction scheme 3. Preferred reaction conditions comprise reacting compounds of formula (IV) with carboxylic acid R^{2a}CO₂H in the presence of EDC and HOBt. Alternatively compounds of formula (Ib) may be prepared by reacting compounds of formula (IV) with acid chloride R^{2a}COCl in the presence of triethylamine.

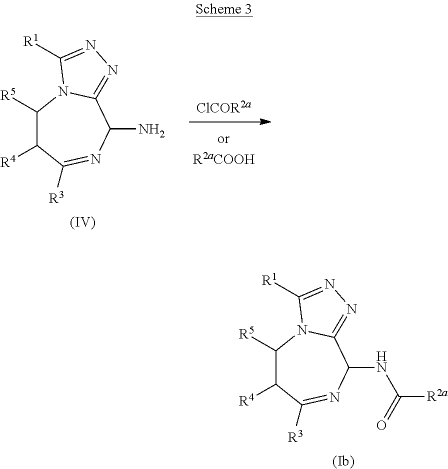

Compounds of formula (Ic), i.e. compounds of formula (I) where R² is —NHR^{2c} and X is O may be prepared according to reaction scheme 4, by reacting compounds of formula (IV) with R^{2c}NCO.

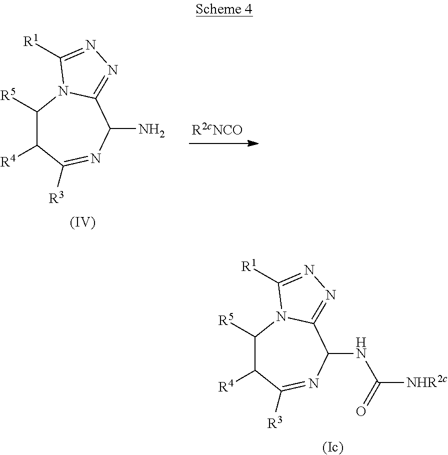

Compounds of formula (Id), i.e. compounds of formula (I) where R² is —NHR^{2c} and X is S may be prepared according to reaction scheme 5, by reacting compounds of formula (IV) with R^{2c}NCS.

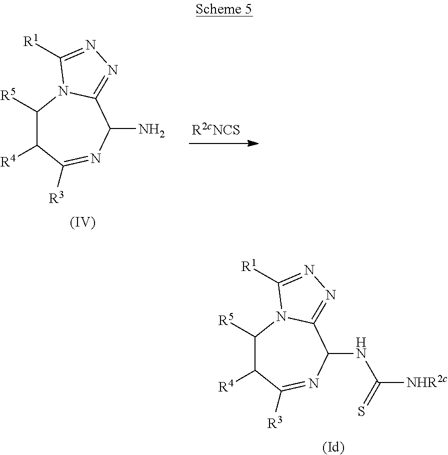

Compounds of formula (IV) may be prepared according to reaction scheme 6, by reacting compounds of formula (Ie), i.e. compounds of formula (I) where R² is benzyloxy and X is O with palladium on charcoal in either a hydrogen atmosphere or in presence of cyclohexadiene.

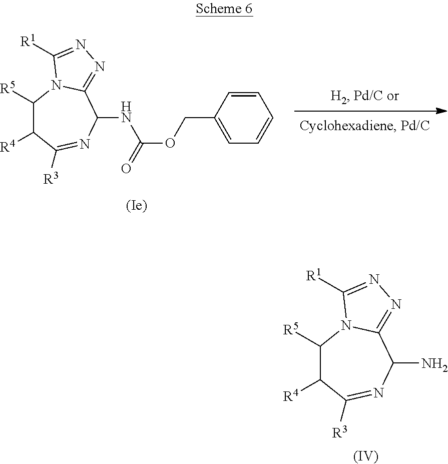

Compounds of formula (IV) may also be prepared according to reaction scheme 7, by reacting compounds of formula (If), i.e. compounds of formula (I) where R² is tert-butoxy and X is O with trifluoroacetic acid in refluxing dicholoromethane.

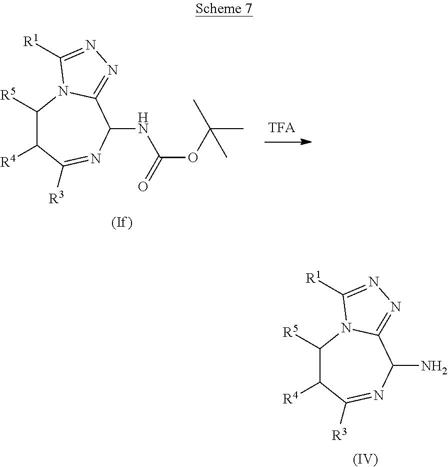

Compounds of formula (III) may be prepared according to reaction scheme 8 from compounds of formula (V) by treatment with Lawesson's reagent or P₄S₁₀. Preferred reaction conditions comprise reacting Intermediate (V) with Lawesson's reagent in refluxing toluene.

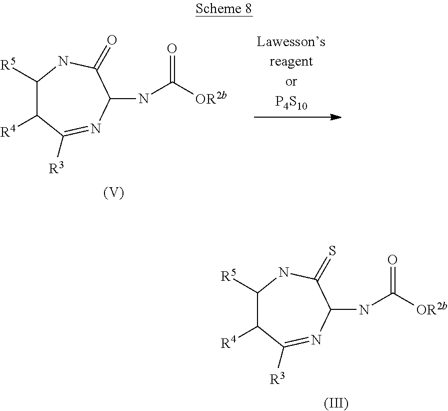

Alternatively compounds of formula (V) may be prepared according to reaction scheme 9, by reacting compounds of formula (VI) with compounds of formula ClCOOR^{2b} or compounds of formula O(COOR^{2b})₂ in the presence of triethylamine.

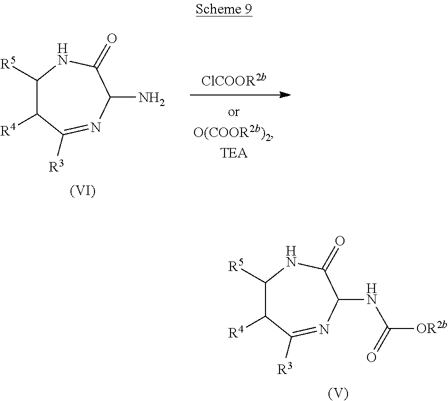

Compounds of formula (VI), may be prepared according to reaction scheme 10 from compounds of formula (Va), i.e. compounds of formula (V) where R² is benzyloxy, at 80° C., by treatment with hydrogen bromide in acetic acid.

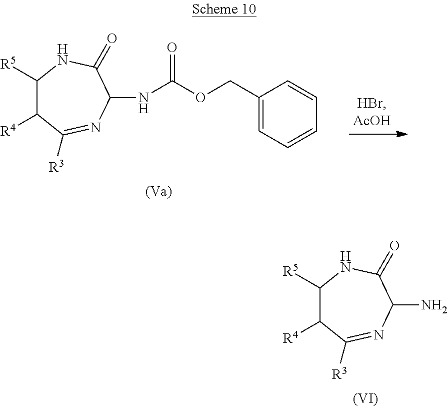

Compounds of formula (V), may be prepared according to reaction scheme 11 from compounds of formula (VII), at room temperature, by treatment with ammonium acetate in acetic acid.

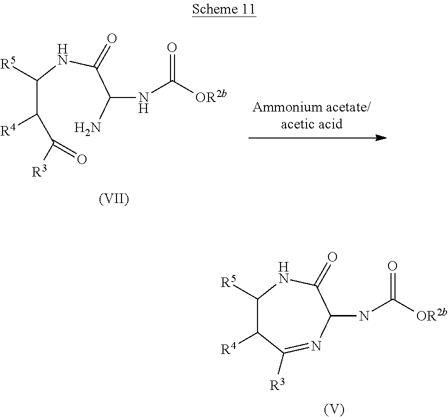

Compounds of formula (VII), may be prepared according to reaction scheme 12 from compounds of formula (VIII) at room temperature, by treatment with a methanolic solution of ammonia.

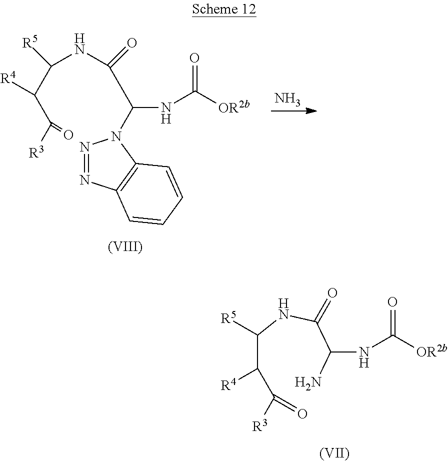

Compounds of formula (VIII), may be prepared according to reaction scheme 13 from Intermediates (IX) upon treatment with EDC and HOBt or with oxalyl chloride and subsequent coupling reaction with compounds of formula (X) at room temperature.

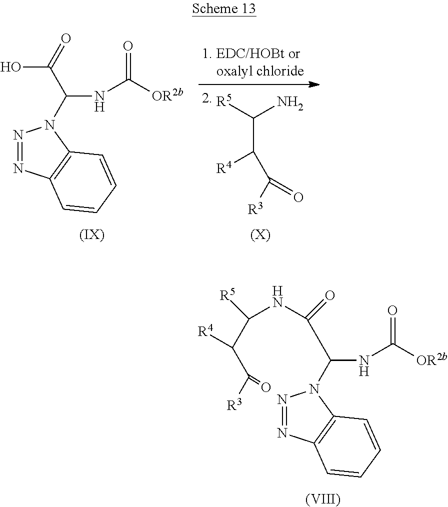

Compounds of formula (IX) may be prepared according to procedures described in J. Org. Chem. 1990, 55, 2206.

It will be appreciated by those skilled in the art that it may be advantageous to protect one or more functional groups of the compounds described in the above processes. Examples of protecting groups and the means for their removal can be found in T. W. Greene ‘Protective Groups in Organic Synthesis’ (4th edition, J. Wiley and Sons, 2006). Suitable amine protecting groups include acyl (e.g. acetyl, carbamate (e.g. 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxane or trifluoroacetic acid in dichloromethane) or reductively (e.g. hydrogenolysis of a benzyl or benzyloxycarbonyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis.

It will be appreciated that in any of the routes described above, the precise order of the synthetic steps by which the various groups and moieties are introduced into the molecule may be varied. It will be within the skill of the practitioner in the art to ensure that groups or moieties introduced at one stage of the process will not be affected by subsequent transformations and reactions, and to select the order of synthetic steps accordingly.

Certain intermediate compounds described above are believed to be novel and therefore form a yet further aspect of the invention.

The compounds of formula (I) and salts thereof are bromodomain inhibitors, and thus are believed to have potential utility in the treatment of diseases or conditions for which a bromodomain inhibitor is indicated.

The present invention thus provides a compound of formula (I) or a pharmaceutically acceptable salt thereof for use in therapy. The compound of formula (I) or pharmaceutically salt thereof can be for use in the treatment of diseases or conditions for which a bromodomain inhibitor indicated.

In one embodiment there is provided a compound of formula (I) or a pharmaceutically acceptable salt thereof for use in the treatment of any diseases or conditions for which a bromodomain inhibitor is indicated. In another embodiment, there is provided a compound or a pharmaceutically acceptable salt thereof for use in the treatment of a chronic autoimmune and/or inflammatory condition. In a further embodiment, there is provided a compound or a pharmaceutically acceptable salt thereof for use in the treatment of cancer, such as midline carcinoma.

In one embodiment there is provided the use of a compound of formula (I) or a pharmaceutically acceptable salt thereof in the manufacture of a medicament for the treatment of diseases or conditions for which a bromodomain inhibitor is indicated. In another embodiment, there is provided the use of a compound of formula (I) or a pharmaceutically acceptable salt thereof in the manufacture of a medicament for the treatment of a chronic autoimmune and/or inflammatory condition. In a further embodiment, there is provided the use of a compound of formula (I) or a pharmaceutically acceptable salt thereof in the manufacture of a medicament for the treatment of cancer, such as midline carcinoma.

In one embodiment there is provided a method of treating diseases or conditions for which a bromodomain inhibitor is indicated, in a subject in need thereof which comprises administering a therapeutically effective amount of compound of formula (I) or a pharmaceutically acceptable salt thereof. In another embodiment there is provided a method for treatment of a chronic autoimmune and/or inflammatory condition, in a subject in need thereof which comprises administering a therapeutically effective amount of compound of formula (I) or a pharmaceutically acceptable salt thereof. In a further embodiment there is provided a method for treatment of cancer, such as midline carcinoma, in a subject in need thereof which comprises administering a therapeutically effective amount of compound of formula (I) or a pharmaceutically acceptable salt thereof.

In one embodiment the subject in need thereof is a mammal, particularly a human.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Bromodomain inhibitors are believed to be useful in the treatment of a variety of diseases or conditions related to systemic or tissue inflammation, inflammatory responses to infection or hypoxia, cellular activation and proliferation, lipid metabolism, fibrosis and in the prevention and treatment of viral infections.

Bromodomain inhibitors may be useful in the treatment of a wide variety of chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), asthma, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, depression, retinitis, uveitis, scleritis, hepatitis, pancreatitis, primary biliary cirrhosis, sclerosing cholangitis, Addison's disease, hypophysitis, thyroiditis, type I diabetes, acute rejection of transplanted organs.

Bromodomain inhibitors may be useful in the treatment of a wide variety of acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement, acute rejection of transplanted organs.

Bromodomain inhibitors may be useful in the prevention or treatment of diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria, SIRS associated with viral infections such as influenza, herpes zoster, herpes simplex, coronavirus.

Bromodomain inhibitors may be useful in the prevention or treatment of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

Bromodomain inhibitors may be useful in the treatment of disorders of lipid metabolism via the regulation of APO-A1 such as hypercholesterolemia, atherosclerosis and Alzheimer's disease.

Bromodomain inhibitors may be useful in the treatment of fibrotic conditions such as idiopathic pulmonary fibrosis, renal fibrosis, post-operative stricture, keloid formation, scleroderma, cardiac fibrosis.

Bromodomain inhibitors may be useful in the prevention and treatment of viral infections such as herpes virus, human papilloma virus, adenovirus and poxvirus and other DNA viruses.

Bromodomain inhibitors may be useful in the treatment of cancer, including hematological, epithelial including lung, breast and colon carcinomas, mesenchymal, hepatic, renal and neurological tumours.

In one embodiment the disease or condition for which a bromodomain inhibitor is indicated is selected from diseases associated with systemic inflammatory response syndrome, such as sepsis, burns, pancreatitis, major trauma, haemorrhage and ischaemia. In this embodiment the bromodomain inhibitor would be administered at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality. In another embodiment the bromodomain inhibitor would be administered prior to surgical or other procedures associated with a high risk of sepsis, haemorrhage, extensive tissue damage, SIRS or MODS (multiple organ dysfunction syndrome). In a particular embodiment the disease or condition for which a bromodomain inhibitor is indicated is sepsis, sepsis syndrome, septic shock and endotoxaemia. In another embodiment, the bromodomain inhibitor is indicated for the treatment of acute or acute on chronic pancreatitis. In another embodiment the bromodomain inhibitor is indicated for the treatment of burns.

In one embodiment the disease or condition for which a bromodomain inhibitor is indicated is selected from herpes simplex infections and reactivations, cold sores, herpes zoster infections and reactivations, chickenpox, shingles, human papilloma virus, cervical neoplasia, adenovirus infections, including acute respiratory disease, poxvirus infections such as cowpox and smallpox and African swine fever virus. In one particular embodiment a bromodomain inhibitor is indicated for the treatment of Human papilloma virus infections of skin or cervical epithelia.

The term “diseases or conditions for which a bromodomain inhibitor is indicated”, is intended to include any of or all of the above disease states.

In one embodiment, there is provided a method for inhibiting a bromodomain which comprises contacting the bromodomain with a compound of formula (I) or a pharmaceutically acceptable salt thereof.

While it is possible that for use in therapy, a compound of formula (I) as well as pharmaceutically acceptable salts thereof may be administered as the raw chemical, it is common to present the active ingredient as a pharmaceutical composition.

The present invention therefore provides in a further aspect a pharmaceutical composition comprising a compound of formula (I) or a pharmaceutically acceptable salt and one or more or pharmaceutically acceptable carriers, diluents or excipients. The compounds of the formula (I) and pharmaceutically acceptable salts thereof, are as described above. The carrier(s), diluent(s) or excipient(s) must be acceptable in the sense of being compatible with the other ingredients of the composition and not deleterious to the recipient thereof. In accordance with another aspect of the invention there is also provided a process for the preparation of a pharmaceutical composition including admixing a compound of the formula (I), or a pharmaceutically acceptable salt thereof, with one or more pharmaceutically acceptable carriers, diluents or excipients. The pharmaceutical composition can be for use in the treatment of any of the conditions described herein.

Since the compounds of formula (I) are intended for use in pharmaceutical compositions it will be readily understood that they are each preferably provided in substantially pure form, for example, at least 60% pure, more suitably at least 75% pure and preferably at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

In one embodiment there is provided a pharmaceutical composition comprising a compound of formula (I) or a pharmaceutically acceptable salt thereof adapted for oral administration.

In one embodiment the pharmaceutical composition is adapted for parenteral administration, particularly intravenous administration.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The compounds of formula (I) and pharmaceutically acceptable salts thereof can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Dosage forms for nasal or inhaled administration may conveniently be formulated as aerosols, solutions, suspensions, gels or dry powders.

For compositions suitable and/or adapted for inhaled administration, it is preferred that the compound of formula (I) or a pharmaceutically acceptable salt thereof, is in a particle-size-reduced form e.g. obtained by micronisation. The preferable particle size of the size-reduced (e.g. micronised) compound or salt is defined by a D50 value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the compound of formula (I) or salt thereof (preferably in particle-size-reduced form, e.g. in micronised form), and optionally a performance modifier such as L-leucine or another amino acid and/or metals salts of stearic acid such as magnesium or calcium stearate. Preferably, the dry powder inhalable composition comprises a dry powder blend of lactose e.g. lactose monohydrate and the compound of formula (I) or salt thereof. Such compositions can be administered to the patient using a suitable device such as the DISKUS® device, marketed by GlaxoSmithKline which is for example described in GB 2242134A.

The compounds of formula (I) thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO2005/044354 A1.

A therapeutically effective amount of a compound formula (I) or a pharmaceutically acceptable salt thereof will depend upon a number of factors including, for example, the age and weight of the animal, the precise condition requiring treatment and its severity, the nature of the formulation, and the route of administration, and will ultimately be at the discretion of the attendant physician or veterinarian. In the pharmaceutical composition, each dosage unit for oral or parenteral administration preferably contains from 0.01 to 3000 mg, more preferably 0.5 to 1000 mg, of a compound of formula (I) or a pharmaceutically acceptable salt thereof, calculated as the free base. Each dosage unit for nasal or inhaled administration preferably contains from 0.001 to 50 mg, more preferably 0.01 to 5 mg, of a compound of the formula (I) or a pharmaceutically acceptable salt thereof, calculated as the free base.

The pharmaceutically acceptable compounds the invention can be administered in a daily dose (for an adult patient) of, for example, an oral or parenteral dose of 0.01 mg to 3000 mg per day or 0.5 to 1000 mg per day, or a nasal or inhaled dose of 0.001 to 50 mg per day or 0.01 to 5 mg per day, of the compound of the formula (I) or a pharmaceutically acceptable salt thereof, calculated as the free base. This amount may be given in a single dose per day or more usually in a number (such as two, three, four, five or six) of sub-doses per day such that the total daily dose is the same. An effective amount of a pharmaceutically acceptable salt thereof, may be determined as a proportion of the effective amount of the compound of formula (I) per se.

The compounds of formula (I) and pharmaceutically acceptable salts thereof may be employed alone or in combination with other therapeutic agents. Combination therapies according to the present invention thus comprise the administration of at least one compound of formula (I) or a pharmaceutically acceptable salt thereof, and the use of at least one other pharmaceutically active agent. Preferably, combination therapies according to the present invention comprise the administration of at least one compound of formula (I) or a pharmaceutically acceptable salt thereof, and at least one other pharmaceutically active agent. The compound(s) of formula (I) and pharmaceutically acceptable salts thereof, and the other pharmaceutically active agent(s) may be administered together in a single pharmaceutical composition or separately and, when administered separately this may occur simultaneously or sequentially in any order. The amounts of the compound(s) of formula (I) and pharmaceutically acceptable salts thereof, and the other pharmaceutically active agent(s) and the relative timings of administration will be selected in order to achieve the desired combined therapeutic effect. Thus in a further aspect, there is provided a combination comprising a compound of formula (I) or a pharmaceutically acceptable salt thereof, and at least one other pharmaceutically active agent. In one embodiment there is provided a combination pharmaceutical product comprising a compound of formula (I) or a pharmaceutically acceptable salt thereof together with one or more other therapeutically active agents.

Thus in one aspect, the compound and pharmaceutical compositions according to the invention may be used in combination with or include one or more other therapeutic agents, for example selected from antibiotics, anti-virals, glucocorticosteroids, muscarinic antagonists and beta-2 agonists.

It will be appreciated that when the compound of the present invention is administered in combination with other therapeutic agents normally administered by the inhaled, intravenous, oral or intranasal route, that the resultant pharmaceutical composition may be administered by the same routes. Alternatively the individual components of the composition may be administered by different routes.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The compounds of formula (I) and salts thereof may be prepared by the methods described below or by similar methods. Thus the following Intermediates and Examples serve to illustrate the preparation of the compounds of formula (I) and salts thereof, and are not to be considered as limiting the scope of the invention in any way.

### General Experimental Details

All temperatures referred to are in ° C.

### ABBREVIATIONS

SPE—solid phase extraction
TLC—thin layer chromatography
AcOH—acetic acid
DCM—dichloromethane
4-DMAP—4-dimethylaminopyridine
DMF—N,N-dimethylformamide
EDC—1-(3-dimethylaminopropyl)-3-ethylcarbodiimide hydrochloride
Et₂O—diethyl ether
EtOAc—ethyl acetate
HOBt—1-hydroxybenzotriazole
Lawesson's—2,4-bis(4-methoxyphenyl)-1,3-dithia-2,4-diphosphetane-2,4-disulphide Reagent
MeCN—acetonitrile
MeOH—methanol
NMM—N-methylmorpholine
Rt—retention time
TBTU—2-[1H-benzotriazol-1-yl]-1,1,3,3-tetramethyluronium terafluoroborate
THF—tetrahydrofuran
RT—room temperature
TEA—triethylamine
DIPEA—N,N-diisopropylethylamine

LC/MS refers to analyses by analytical HPLC which were conducted on two kinds of apparatus:
a) On a Supelcosil LCABZ+PLUS column (3 μm, 3.3 cm×4.6 mm ID) eluting with 0.1% HCO₂H and 0.01 M ammonium acetate in water (solvent A), and 95% acetonitrile and 0.05% HCO₂H in water (solvent B), using the following elution gradient 0-0.7 minutes 0% B, 0.7-4.2 minutes 0→100%/0B, 4.2-5.3 minutes 100% B, 5.3-5.5 minutes 100→0% B at a flow rate of 3 mL/minute. The mass spectra (MS) were recorded on a Fisons VG Platform mass spectrometer using electrospray positive ionisation [(ES+ve to give [M+H]⁺ and [M+NH₄]⁺ molecular ions] or electrospray negative ionisation [(ES−ve to give [M−H]⁻ molecular ion] modes. Analytical data from this apparatus are given with the following format: [M−H]⁻ or [M−H]⁻.
b) On a Chromolith Performance RP 18 column (100×4.6 mm id) eluting with 0.01 M ammonium acetate in water (solvent A) and 100% acetonitrile (solvent B), using the following elution gradient 0-4 minutes 0Π 100% B, 4-5 minutes 100% B at a flow rate of 5 mL/minute. The mass spectra (MS) were recorded on a micromass Platform-LC mass spectrometer using atmospheric pressure chemical positive ionisation [AP+ve to give MH⁺ molecular ions] or atmospheric pressure chemical negative ionisation [AP−ve to give (M−H)⁻ molecular ions] modes. Analytical data from this apparatus are given with the following format: [M+H]⁺ or [M−H]⁻ preceded by the acronym APCI to specify between both mass spectrometry analyses sources.
c) On a Waters Acquity HPLC BEH C18 column (2 mm×50 mm id, 1.7 μm packing diameter) at 50° C., eluting with 0.2% v/v solution of formic acid in water (solvent system A) and 0.15% v/v solution of formic acid in acetonitrile (solvent system B). The gradient employed was:

| | Flow Rate | Solvent System | |
|---|---|---|---|
| Time (min) | (ml/min) | % A | % B |
| 0 | 1 | 95 | 5 |
| 1.1 | 1 | 1 | 99 |
| 1.5 | 1 | 1 | 99 |

The UV detection system was an averaged signal from wavelength of 210 nm to 350 nm and mass spectra were recorded on a mass spectrometer using alternate-scan positive and negative mode electrospray ionization.

LC/HRMS: Analytical HPLC was conducted on a Uptisphere-hsc column (3 μm 33×3 mm id) eluting with 0.01M ammonium acetate in water (solvent A) and 100% acetonitrile (solvent B), using the following elution gradient 0-0.5 minutes 5% B, 0.5-3.75 minutes 5Π100% B, 3.75-4.5 100% B, 4.5-5 100Π5% B, 5-5.5 5% B at a flow rate of 1.3 mL/minute. The mass spectra (MS) were recorded on a micromass LCT mass spectrometer using electrospray positive ionisation [ES+ve to give MH⁺ molecular ions] or electrospray negative ionisation [ES−ve to give (M−H)- molecular ions] modes.

Biotage™ chromatography refers to purification carried out using equipment sold by Dyax Corporation (either the Flash 40i or Flash 150i) and cartridges pre-packed with KP-SiITM silica.

Mass directed auto-prep HPLC refers to the method where the material was purified by high performance liquid chromatography on a HPLCABZ+5 μm column (5 cm×10 mm i.d.) with 0.1% HCO₂H in water and 95% MeCN, 5% water (0.5% HCO₂H) utilising the following gradient elution conditions: 0-1.0 minutes 5% B, 1.0-8.0 minutes 5-30% B, 8.0-8.9 minutes 30% B, 8.9-9.0 minutes 30→95% B, 9.0-9.9 minutes 95% B, 9.9-10 minutes 95→0% B at a flow rate of 8 mL/minute. The Gilson 202-fraction collector was triggered by a VG Platform Mass Spectrometer on detecting the mass of interest.

Proton NMR (¹H NMR) spectra were recorded at ambient temperature on a Bruker Avance 300 DPX spectrometer using solvent as internal standard and proton chemical shifts are expressed in ppm in the indicated solvent. The following abbreviations are used for multiplicity of NMR signals: s=singlet, d=doublet, t=triplet, q=quadruplet, dd=double doublet, m=multiplet.

SPE (solid phase extraction) refers to the use of cartridges sold by International Sorbent Technology Ltd. SCX is a benzene sulfonic acid stationary phase.

TLC (thin layer chromatography) refers to the use of TLC plates sold by Merck coated with silica gel 60 F254.

### Example 1

### phenylmethyl [6-(4-fluorophenyl)-1-methyl-4H-[1,2,4]triazolo[4,3-a][1,4]benzodiazepin-4-yl]carbamate

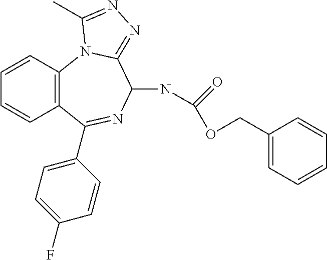

To a solution of Intermediate 1 (204 mg, 0.49 mmol) in dry methanol (3.3 mL) under a nitrogen atmosphere was added hydrazine monohydrate (0.36 mL, 7.3 mmol) and the solution was stirred at room temperature for 2 hours. The solvent was removed under reduced pressure and the residue was dissolved in DCM (15 mL). The organic phase was washed twice with water and concentrated. Under an atmosphere of nitrogen, dry THF (2 mL) was added and the mixture was cooled to 0° C. in an ice bath. DIPEA (0.085 mL, 0.51 mmol) and acetyl chloride (0.036 mL, 5.11 mmol) were added and the mixture was stirred for 30 mins. The solvent was removed in vacuo, the residue dissolved in acetic acid (4.1 mL) and the mixture stirred at reflux for 30 mins. The solvent was removed in vacuo to give a residue which was dissolved in chloroform and washed with saturated NaHCO₃. The organic phase was added to a 10 Si SPE cartridge conditioned with chloroform. The cartridge was washed with 1:1 EtOAc:cyclohexane, then 3:1 EtOAc:cyclohexane and product was eluted with EtOAc. The solvent was removed and product freeze-dried from 1,4-dioxane to give the title compound; LC/MS: m/z 442 [M+H]+, Rt=3.09 min.

Examples 2 to 15 of formula (Ia) (see Table 1) were prepared by methods analogous to that described for Example 1 using the Intermediates indicated in the table and the appropriate acylchloride.

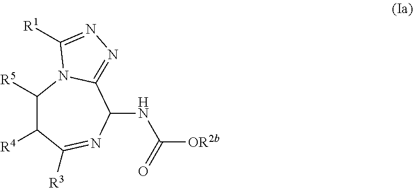

TABLE 1

| Ex | R¹ | R³ | R^{2b} | R⁴/R⁵ | From Int. | Physical data |
|---|---|---|---|---|---|---|
| 2 | Me | 4-Cl-phenyl | benzyl | (R⁴)—(CH)₄—(R⁵) | 2 | LC/MS: m/z |
| | | | | | | 458 [M + H]⁺, |
| | | | | | | Rt = 3.23 min |
| 3 | Et | 4-Cl-phenyl | benzyl | (R⁴)—(CH)₄—(R⁵) | 2 | LC/MS: m/z |
| | | | | | | 396 [M + H]⁺, |
| | | | | | | Rt = 2.27 min |
| 4 | Me | 4-Cl-phenyl | ethyl | (R⁴)—(CH)₄—(R⁵) | 3 | LC/MS: m/z |
| | | | | | | 396 [M + H]⁺, |
| | | | | | | Rt = 2.27 min |
| 5 | Et | 4-Cl-phenyl | ethyl | (R⁴)—(CH)₄—(R⁵) | 3 | LC/MS: m/z |
| | | | | | | 410 [M + H]⁺, |
| | | | | | | Rt = 2.40 min |
| 6 | Me | phenyl | benzyl | (R⁴)—CH—C(OMe)—(CH)₂—(R⁵) | 4 | LC/MS: m/z |
| | | | | | | 454.175 |
| | | | | | | [M + H]⁺, |
| | | | | | | Rt = 2.74 min |
| 7 | Me | 4-MeO-phenyl | benzyl | (R⁴)—(CH)₄—(R⁵) | 5 | LC/MS: m/z |
| | | | | | | 454 [M + H]⁺, |
| | | | | | | Rt = 3.05 min |
| 8 | Me | 4-tolyl | benzyl | (R⁴)—(CH)₄—(R⁵) | 6 | LC/MS: m/z |
| | | | | | | 438 [M + H]⁺, |
| | | | | | | Rt = 3.16 min |
| 9 | Me | 3-MeO-phenyl | benzyl | (R⁴)—(CH)₄—(R⁵) | 7 | LC/MS: m/z |
| | | | | | | 454 [M + H]⁺, |
| | | | | | | Rt = 3.05 min |
| 10 | Me | phenyl | benzyl | (R⁴)—(CH)₂—C(Me)—CH—(R⁵) | 8 | LC/MS: m/z |
| | | | | | | 438 [M + H]⁺, |
| | | | | | | Rt = 3.15 min |
| 11 | Me | phenyl | benzyl | (R⁴)—CH—C(Cl)—(CH)₂—(R⁵) | 9 | LC/MS: m/z |
| | | | | | | 458 [M + H]⁺, |
| | | | | | | Rt = 3.23 min |
| 12 | Me | phenyl | benzyl | (R⁴)—CH—CH—S—(R⁵) | 10 | LC/MS: m/z |
| | | | | | | 430 [M + H]⁺, |
| | | | | | | Rt = 3.00 min |
| 13 | Me | phenyl | benzyl | (R⁴)—S—CH—CH—(R⁵) | 11 | LC/MS: m/z |
| | | | | | | 430 [M + H]⁺, |
| | | | | | | Rt = 2.97 min |
| 14 | Me | 4-Cl-phenyl | benzyl | (R⁴)—CH—C(OMe)—(CH)₂—(R⁵) | 16 | LC/MS: m/z |
| | | | | | | 488 [M + H]⁺, |
| | | | | | | Rt = 2.55 min |
| 15 | Et | phenyl | benzyl | (R⁴)—(CH)₄—(R⁵) | see J. Med. | LC/MS: m/z |
| | | | | | Chem., (1988), | 438 [M + H]⁺, |
| | | | | | 31(1), 176-81 | Rt = 3.11 min |

### Example 16

### phenylmethyl [6-(2-fluorophenyl)-8-iodo-1-methyl-4H-[1,2,4]triazolo[4,3-a][1,4]benzodiazepin-4-yl]carbamate

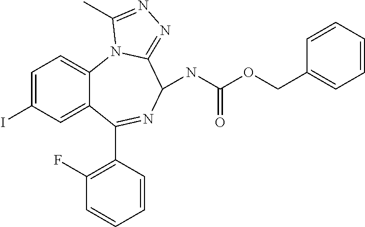

To a solution of Intermediate 17 (15 mg, 0.02 mmol) in dry DCM (3 mL), was added iodine (7.8 mg, 0.03 mmol, 1.5 equiv.), and the mixture was stirred at RT overnight. The organic layer was treated with Na₂S₂O₅ and separated, dried over Na₂SO₄, concentrated, triturated in diisopropyl ether with 2 drops of acetonitrile, filtered and purified by flash chromatography to give the title compound as an orange solid (10 mg); LC/MS, APCI, (M+H)⁺ 568.03, (M−H) 565.98.

### Example 17

### phenylmethyl [8-bromo-6-(2-fluorophenyl)-1-methyl-4H-[1,2,4]triazolo[4,3-a][1,4]benzodiazepin-4-yl]carbamate

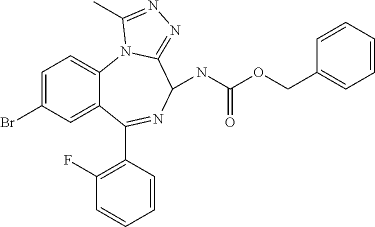

A solution of trimethyl orthoacetate (280 μL, 2.2 mmol) and Intermediate 18 in ethanol (10 mL) was treated with a catalytic amount of concentrated sulfuric acid (2 drops) at room temperature and the mixture was allowed to stir overnight. The reaction mixture was concentrated, the residue was dissolved in DCM, washed with water and brine, dried (Na₂SO₄), filtered and the solvent was evaporated. Diethyl ether was added and the resulting precipitate was filtered to give the title product; m.p. 160-170° C.; HRMS calculated for C₂₅H₁₉BrFN₅O₂ (M+H)⁺: 520.0836; Found: 520.0784.

Examples 18 to 25 of formula (Ia) (see Table 2) were prepared by methods analogous to that described for Example 17 using the Intermediates indicated in the table and the appropriate orthoester. PPTS was used in place of concentrated sulfuric acid for Examples 18, 19, 20, 21, 22, 23, 24 and the reaction was refluxed for 2 h.

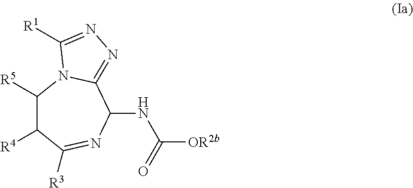

TABLE 2

| | | | | | From | |
|---|---|---|---|---|---|---|
| Ex | R¹ | R³ | R^{2b} | R⁴/R⁵ | Int. | Physical data |
| 18 | Me | phenyl | benzyl | 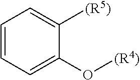 | 19 | HRMS calculated for C₂₇H₂₂N₅O₃ (M + H)⁺ 464.17.22 found 464.1527 |
| 19 | Et | phenyl | benzyl | 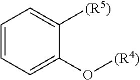 | 19 | LC/MS: m/z 478 [M + H]⁺, Rt = 3.01 min |
| 20 | Me | 4-Cl-phenyl | ethyl | 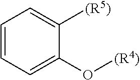 | 20 | HRMS calculated for C₂₇H₁₉ClN₅O₃ (M + H)⁺ 436.1176 found 436.1191 |
| 21 | Et | 4-MeO- | benzyl | (R⁴)—(CH)₄—(R⁵) | 21 | LC/MS: m/z 468 |
| | | phenyl | | | | [M + H]⁺, Rt = 2.57 min |
| 22 | Et | 4-F-phenyl | benzyl | (R⁴)—(CH)₄—(R⁵) | 22 | LC/MS: m/z 456 |
| | | | | | | [M + H]⁺, Rt = 2.61 min |
| 23 | Me | 4-F-phenyl | ethyl | (R⁴)—(CH)₄—(R⁵) | 23 | HRMS (M + H)⁺ |
| | | | | | | calculated for |
| | | | | | | C₂₀H₁₉FN₅O₂ |
| | | | | | | 380.1523 found |
| | | | | | | 380.1514 |
| 24 | Me | phenyl | benzyl | (R⁴)—CH—C(I)—(CH)₂—(R⁵) | 24 | HRMS calculated for |
| | | | | | | C₂₅H₂₁IN₅O₂ (M + H)⁺ |
| | | | | | | 550.0740 found |
| | | | | | | 550.0448 |
| 25 | Me | phenyl | t-butyl | (R⁴)—CH—C(I)—(CH)₂—(R⁵) | 25 | HRMS calculated for |
| | | | | | | C₁₈H₁₅IN₅O₂ (M + H − tBu)⁺ |
| | | | | | | 460.0271 found |
| | | | | | | 460.0056 [M + H − tBu]+ |

### Example 26

### phenylmethyl [6-(2-fluorophenyl)-8-(2-furanyl)-1-methyl-4H-[1,2,4]triazolo[4,3-a][1,4]benzodiazepin-4-yl]carbamate

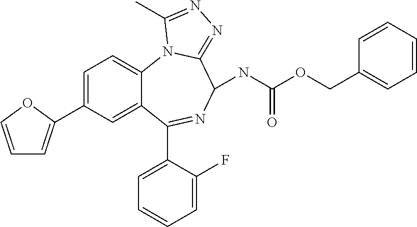

A mixture of Example 17 (0.3 mmole), tributyl(2-furanyl)stannane (535 mg) and Pd(PPh₃)₄ (0.1 equiv) in dry THF (10 mL) was stirred overnight at 40° C. Further tributyl(2-furanyl)stannane (535 mg) and further Pd(PPh₃)₄ (0.1 equiv) were added and the reaction mixture was stirred at 40° C. for a further 6 hours. On cooling, aqueous ammonium chloride solution (50 mL) was added and the mixture was extracted 3 times with DCM (150 mL). The combined organic layers were dried (Na₂SO₄), filtered and concentrated in vacuo to give a residue which was purified by chromatography on silica gel eluting with DCM/MeOH:98/2. Concentration in vacuo and trituration of the residue in a mixture of EtOH/iPrOH/H₂O gave the title compound as a white solid; LC/MS: APCI, m/z 508.14 [M+H]⁺, Rt=2.79 min; HRMS calculated for C₂₉H₂₂FN₅O₃ (M+H)⁺ 508.1785 found 508.1843.

### Example 27

### (+)-phenylmethyl (1-methyl-6-phenyl-4H-[1,2,4]triazolo[4,3-a][1,4]benzodiazepin-4-yl)carbamate

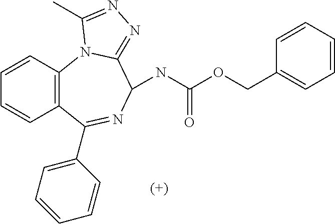

Racemic mixture of phenylmethyl (1-methyl-6-phenyl-4H-[1,2,4]triazolo[4,3-a][1,4]benzo diazepin-4-yl)carbamate [prepared according to the procedure described in the J. Med. Chem., (1988, 31(1), 176-181)] was separated by HPLC using a (R,R) whelk-01 column with Hexane/EtOH as the mobile phase. The sample was prepared in a 80/20 mixture EtOH/Hexane (Note: the sample required heating and filtering prior to addition to the column). The system used for preparative separation was as follows: Column: (R,R) Whel-01 51×250 mm column (2 inch columns); mobile phase: 50/50, Hexane/EtOH; Flow rate: 45.0 mL/min; UV wavelength: 254 nm. The title compound eluted at 49 min as the first peak. [α]_{D}=+44.7 c=1.0525 (g/100 mL)/MeOH. The other enantiomer came off at 58 minutes.

### Example 28

### (+)-ethyl [6-(4-chlorophenyl)-1-methyl-4H-[1,2,4]triazolo[4,3-a][1,4]benzodiazepin-4-yl]carbamate

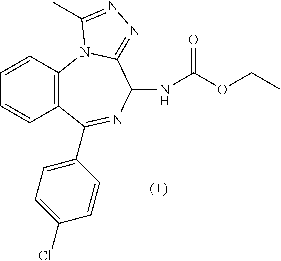

Racemic mixture of ethyl [6-(4-chlorophenyl)-1-methyl-4H-[1,2,4]triazolo[4,3-a][1,4]benzodiazepin-4-yl]carbamate (Example 4) was separated by HPLC using a ChiralPack AD (250*4.6 mm-10 μm) column with Hexane/EtOH as the mobile phase. The sample was prepared in a 60/40 mixture EtOH/Hexane (Note: the sample required heating and filtering prior to addition to the column). The system used for preparative separation was as follows: ChiralPack AD (250*20 mm⁻¹⁰ μm) column; mobile phase: 40/60, Hexane/EtOH; Flow rate: 18.0 mL/min; UV wavelength: 254 nm. The title compound eluted at 16.65 min as the first peak. [α]_{D}=+30.7 c=0.961 (g/100 mL)/MeOH at 25° C. The second enantiomer came off at 35.40 min.

Example 29: ethyl (1-methyl-6-phenyl-4H-[1,2,4]triazolo[4,3-a][1,4]benzodiazepin-4-yl)carbamate

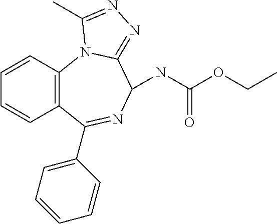

To a solution of Intermediate 28 (140 mg, 0.48 mmole) in dry DCM (10 mL) and pyridine (500 μL) was added one equivalent of ethylchloroformate (46 μL) and the solution was stirred overnight at room temperature. A further equivalent of ethylchloroformate was added and the reaction mixture was allowed to stir at room temperature for an additional 3 hours. The reaction mixture is concentrated in vacuo, dissolved in DCM (150 mL) and washed twice with 1N HCl (30 mL). The organic phase was washed with sodium bicarbonate solution (30 mL) and brine (30 mL), dried (Na₂SO₄), filtered and the filtrate concentrated in vacuo. Addition of diethyl ether gave a a precipitate which was filtered to give the title compound; m.p. 198-202° C.; HRMS (M+H)⁺ calculated for C₂₀H₁₉N₅O₂ 362.1539 found 362.1548.

Examples 30 to 31 of formula (Ig) (see Table 3) were prepared by methods analogous to that described for Example 29 using the Intermediate indicated in the table and triethylamine in the presence of a catalytic amount of DMAP in place of pyridine.

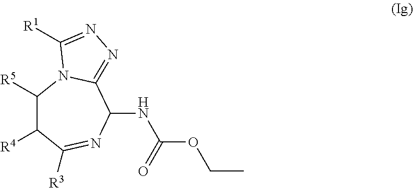

TABLE 3

| Ex | R¹ | R³ | R⁴/R⁵ | From Int. | Physical data |
|---|---|---|---|---|---|
| 30 | Et | 4-F- | (R⁴)—(CH)₄— | 26 | HRMS (M + H)⁺ |
| | | phenyl | (R⁵) | | calculated for |
| | | | | | C₂₁H₂₁FN₅O₂ |
| | | | | | 394.1679 found |
| | | | | | 394.1649 |
| 31 | Me | 4- | (R⁴)—(CH)₄— | 27 | LC/MS: m/z 392 |
| | | MeO- | (R⁵) | | [M + H]+, Rt = 2.03 |
| | | phenyl | | | min |

### Example 32

### (+)-ethyl 1-methyl-6-phenyl-4H-[1,2,4]triazolo[4,3-a][1,4]benzodiazepin-4-ylcarbamate

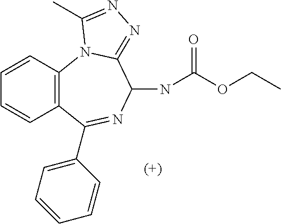

Enantiomers of racemic Example 29 (260 mg) were separated using a 1″ Chiralpak AD column, elution with EtOH/Heptane (80:20), flow rate 15 mL/min. The title compound eluted at Rt 7.5 min (125 mg) as the first eluted isomer [α]_{D}=+46.9, c=0.738 (g/100 mL)/MeOH. The second enantiomer eluted at Rt 13 min.

### Example 33

### 1-(4-Fluorophenyl)ethyl 1-methyl-6-phenyl-4H-[1,2,4]triazolo[4,3-a][1,4]benzodiazepin-4-ylcarbamate

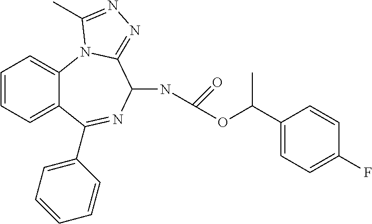

A solution of Intermediate 28 (150 mg, 0.52 mmol), Intermediate 84 (158 mg, 0.52 mmol) and triethylamine (72 μl, 0.52 mmol) in dry MeCN (3 mL) was heated to 75° C. under nitrogen for 4 days. The solvent was evaporated and the residue applied to a 10 g Si SPE cartridge. Elution with cyclohexane, then cyclohexane/DCM (3:1 to 1:1 to 1:3), DCM, DCM/EtOAc (3:1 to 1:1), EtOAc/MeCN (1:1) gave the title compound; 67 mg, 28%; LC/MS: m/z 456 [M+H]⁺, Rt 3.1 min.

Examples 34 to 36 of formula (Ih) were prepared by methods analogous to that described for Example 33 using the starting materials indicated (see Table 4).

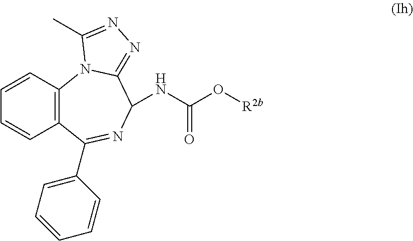

TABLE 4

| | | From | |
|---|---|---|---|
| Ex | R^{2b} | Int. | Physical data |
| 34 | 4-F-phenylmethoxy | 85 | LC/MS: m/z 442 |
| | | | [M + H]⁺, Rt 3.07 min. |
| 35 | 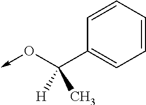 | 86 | Purification by Biotage ™ chromatography (Silica, 40 g) eluting with 1:3 cyclohexane/ EtOAc Second diastereomer: LC/MS: m/z 438 [M + H]⁺, Rt 3.13 min. |
| 36 | 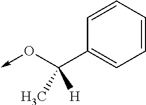 | 87 | Purification by Biotage ™ chromatography (Silica, 40 g) eluting with 1:3 cyclohexane/ EtOAc First diastereomer: LC/MS: m/z 438 [M + H]⁺, Rt 3.09 min. |

### Example 37

### cyclohexyl (1-methyl-6-phenyl-4H-[1,2,4]triazolo[4,3-a][1,4]benzodiazepin-4-ylcarbamate

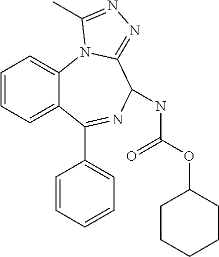

To a solution of 1,1′-carbonyldiimidazole (0.062 g, 0.038 mmol, 1.1 equiv) in THF (0.5 mL) was added Intermediate 28 (0.010 g, 0.035 mmol, 1 equiv) and the mixture was stirred for 16 h under an atmosphere of nitrogen. Cyclohexanol (0.021 g, 0.210 mmol, 5.5 equiv) was added and the mixture was refluxed for 48 h under an atmosphere of nitrogen. The crude material was purified using a Si-SPE cartridge (eluting DCM/MeOH, 95/5), then further purified by mass directed preparative HPLC to give the title compound as a white solid (0.006 g, 42%); LC/MS: m/z 416.20 [M+H]⁺, Rt 3.11 min.

Examples 38 to 45 of formula (Ih) were prepared by methods analogous to that described for Example 37 using the Intermediate 28 and the corresponding alcohol indicated (see Table 5). DIPEA was used in place of pyridine for Example 45, and Examples 38-44 were purified using preparative HPLC in preference to mass directed preparative HPLC.

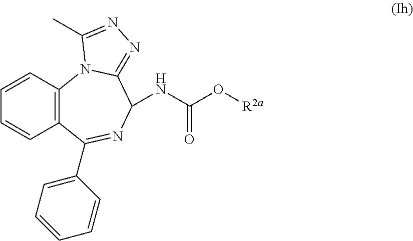

TABLE 5

| Ex | R^{2a} | From Alcohol | [M + H]⁺ | Rt/min |
|---|---|---|---|---|
| 38 | 3-(NMe₂)benzyl | [3-(dimethyl- | 467.36 | 2.94 |
| | | amino)phenyl]- | | |
| | | methanol | | |
| 39 | Me | methanol | 348.31 | 2.53 |
| 40 | CF₃CH₂— | 2,2,2-trifluoro- | 416.26 | 2.93 |
| | | ethanol | | |
| 41 | 2-(imidazol-1-yl)ethyl | 2-(imidazol-1-yl)- | 428.18 | 2.06 |
| | | ethanol | | |
| 42 | 2-(4-Me-thiazol-5- | 2-(4-Me-thiazol- | 459.30 | 2.69 |
| | yl)ethyl | 5-yl)ethanol | | |
| 43 | 2-thienylmethyl | 2- | 430.24 | 3.00 |
| | | thienylmethanol | | |
| 44 | 2-furylmethyl | 2-furylmethanol | 414.31 | 2.90 |
| 45 | 4-methoxybenzyl | [4-(methyloxy)- | 454.29 | 3.04 |
| | | phenyl]methanol | | |

### Example 46

### 2-pyridinylmethyl (1-methyl-6-phenyl-4H-[1,2,4]triazolo[4,3-a][1,4]benzodiazepin-4-yl)carbamate

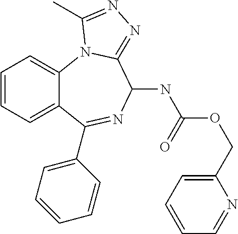

To a solution of 2-pyridinylmethanol (0.011 g, 0.101 mmol, 1.2 equiv) in THF (1 mL) at 0° C. (ice bath) was added a solution of triphosgene (0.010 g, 0.034 mmol, 0.4 equiv) and pyridine (0.010 g, 0.130 mmol, 1.5 equiv) in THF (0.5 mL) and the mixture was stirred for 2.5 h and then allowed to warm to room temperature under an atmosphere of nitrogen. Intermediate 28 (0.025 g, 0.087 mmol, 1 equiv) in THF (2 mL) was added dropwise and the mixture stirred for a further 16 h. PS-TsCl resin was added (0.140 g, 0.280 mmol, 3.2 equiv) and the mixture heated at 50° C. for 2 h, then allowed to cool to room temperature for a further 16 h with stirring. The reaction mixture was filtered, the resin washed with THF (3×8 mL) and the crude material concentrated by vacuum centrifuge. The residue was purified by preparative h.p.l.c. to give the title compound (0.005 g, 14%) as a white solid; LC/MS: m/z 425.29, Rt 2.59 min.

Examples 47 to 52 of formula (Ih) were prepared by methods analogous to that described for Example 46 using the Intermediate 28 and the corresponding alcohol indicated (see Table 6).

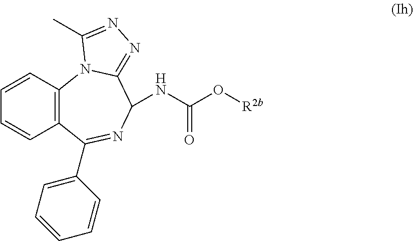

TABLE 6

| Examples | R^{2b} | From Int. | [M + H]⁺ | Rt /min |
|---|---|---|---|---|
| 47 | 4-Cl-benzyl | (4-chloro- | 406.31 | 2.62 |
| | | phenyl)- | | |
| | | methanol | | |
| 48 | cyclopentylmethyl | cyclopentyl- | 416.32 | 3.14 |
| | | methanol | | |
| 49 | cyclopentyl | cyclopentanol | 402.34 | 2.97 |
| 50 | 2-(cyclopropyl)ethyl | 2-cyclo- | 402.34 | 2.99 |
| | | propylethanol | | |
| 51 | 2-(phenyl)ethyl | 2-(phenyl)- | 438.30 | 3.08 |
| | | ethanol | | |
| 52 | cyclobutylmethyl | cyclobutyl- | 402.34 | 3.03 |
| | | methanol | | |

### Example 53

### 6-(methyloxy)-N-(1-methyl-6-phenyl-4H-[1,2,4]-triazolo[4,3-a][1,4]benzodiazepin-4-yl)-1H-indole-2-carboxamide

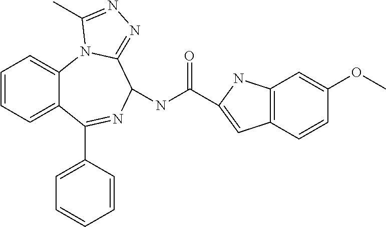

A solution of HOBt (135 mg, 1 mmol), EDC (191 mg, 1 mmol), N,N-diisopropylethylamine (140 μl) and 6-(methoxy)-1H-indole-2-carboxylic acid (Aldrich) (96 mg, 0.6 mmole) in dry THF (10 mL) was stirred at RT for 10 min. Intermediate 28 (145 mg, 0.5 mmol) in DCM (10 mL) was added and stirred for 24 hours. DCM (100 mL) and 8% sodium bicarbonate solution (1 mL) were then added and the organic phase was dried (Na₂SO₄). The mixture was filtered and concentrated in vacuo to give a residue that was triturated with water to give a precipitate which was filtered and washed with iPr₂O (20 mL). Recrystallisation from acetonitrile gave the title compound; m.p. 160-170° C., LC/MS: APCI m/z 463.32 [M+H]⁺, Rt=2.79 min.

Examples 54 to 59 of formula (II) (Table 7) were prepared by methods analogous to that described for Example 53 using the Intermediate 28 and the appropriate carboxylic acides indicated. Triethylamine in the presence of HOBT was used in place of N,N-diisopropylethylamine in example 59.

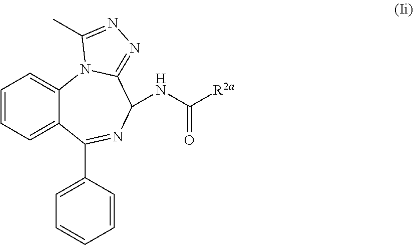

TABLE 7

| Ex | R²a | from Int | Physical data |
|---|---|---|---|
| 54 | 4- | 4-(phenylcarbonyl) | LC/MS: APCl m/z |
| | (phenylcarbonyl) | benzoic acid | 498.22 [M + H]⁺, Rt = |
| | phenyl | (Aldrich) | 2.52 min |
| 55 | benzo[b] | 1-benzofuran-2- | HRMS (M + H)⁺ |
| | furan-2-yl | carboxylic acid | calculated for |
| | | (Aldrich ) | C₂₆H₂₀N₅O₂ 434.4770 |
| | | | found 434.1725 |
| 56 | 2-(4-MeO- | (2E)-3-[4-(methyl- | LC/MS: m/z 450 [M + H]⁺ |
| | phenyl)ethenyl | oxy)phenyl]-2- | Rt 3.0 min. |
| | | propenoic acid | |
| | | (Aldrich) | |
| 57 | 2-(4-Cl- | (2E)-3-(4-chloro- | LC/MS: m/z 454 [M + H]⁺, |
| | phenyl)ethenyl | phenyl)-2- | Rt 3.2 min. |
| | | propenoic acid | |
| | | (Aldrich) | |
| 58 | 2-(thien-2-yl) | (2E)-3-(2-thienyl)- | LC/MS: m/z 426 [M + H]⁺, |
| | ethenyl | 2-propenoic | Rt 2.9 min. |
| | | acid (Aldrich ) | |
| 59 | 4-azidophenyl | 4-Azidobenzoic | HRMS (M + H)⁺ |
| | | acid (TCI-US) | calculated for |
| | | | C₂₄H₁₉N₈O 435.1682 |
| | | | found 435.1677 |

### Example 60

### 5-(methyloxy)-N-(1-methyl-6-phenyl-4H-[1,2,4]triazolo[4,3-a][1,4]benzodiazepin-4-yl)-1-benzothiophene-2-carboxamide

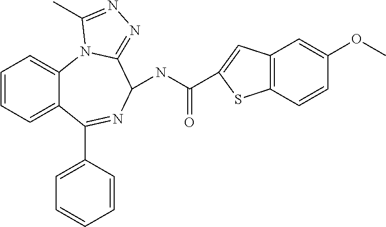

To a solution of 5-(methyloxy)-1-benzothiophene-2-carboxylic acid [see Tetrahedron, 1969, 25(14), 2781-2785] (31 mg, 150 μmol) in anhydrous THF (2 mL) was added of PS carbodiimide argonaut resin (230 mg, 1.31 mmole/g) and the mixture was stirred for 15 minutes. Intermediate 28 (30 mg, 100 μmol) was added and the mixture stirred at room temperature for 16 hours. The mixture was filtered, concentrated in vacuo and the residue was purified by flash chromatography eluting with DCM/MeOH 95/5. Trituration of the residue from diisopropyl ether gave the title compound; LC/MS: APCI m/z 480.13 [M+H]⁺, Rt=2.53 min; ¹H NMR (300 MHz, CDCl₃) δ ppm: 8.17 (d, 1H); 7.91 (s, 1H); 7.68 (m, 2H); 7.52-7.23 (m, 11H); 7.04 (dd, 1H); 6.30 (d, 1H); 3.83 (s, 3H); 2.63 (s, 3H).

Examples 61 to 63 of formula (II) (see table 8) were prepared by methods analogous to that described for Example 60 using the Intermediate 28 and the appropriate carboxylic acides indicated.

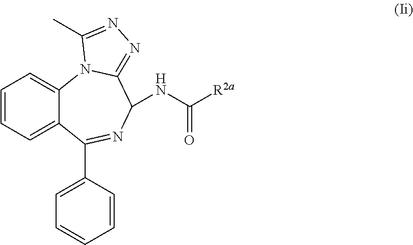

TABLE 8

| Ex | R^{2a} | from Int | Physical data |
|---|---|---|---|
| 61 | 5-Me- | 5-methyl- | LC/MS: APCl m/z 447.13 |
| | indol-2-yl | 1H-indole-2- | [M + H]⁺, Rt 2.53 |
| | | carboxylic acid | |
| | | (Lancaster) | |
| 62 | benzothio- | 1-benzothio- | LC/MS: APCl m/z 450.09 |
| | phen-2-yl | phene-2- | [M + H]⁺, Rt 2.49 |
| | | carboxylic acid | |
| | | (Aldrich) | |
| 63 | 5-MeO-2- | 6-(methyloxy)-2- | HRMS (M + H)⁺ calculated for |
| | naphthyl | naphthalene- | C₂₉H₂₃N₅O₂ 474.1930 found |
| | | carboxylic acid | 474.1825 |
| | | (Lancaster) | |

### Example 64

### (2E)-N-(1-methyl-6-phenyl-4H-[1,2,4]triazolo[4,3-a][1,4]benzodiazepin-4-yl)-3-phenyl-2-propenamide

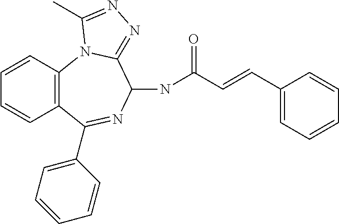

HOBt (23 mg, 0.17 mmol), TBTU (55 mg, 0.17 mmol) and N,N diisopropylethylamine (59 μl, 0.34 mmol, 2 equiv.) were added to (2E)-3-phenylprop-2-enoic acid (Aldrich, 27 mg, 0.17 mmol) in DMF (dry, 2.5 mL) at RT and the mixture was stirred for 10 min. Intermediate 28 (50 mg, 0.17 mmol) was added and the mixture stirred for 5 h. The solvent was evaporated and the residue was dissolved in the minimum volume of DCM and purified using a 5 g Si SPE cartridge. Elution with DCM increasing to DCM/EtOAc (3:1 to 1:1 to 1:3) then EtOAc gave the title compound as a white solid (60 mg, 83%); LC/MS: m/z 420 [M+H]⁺, Rt 3.0 min.

### Example 65

### (2E)-3-(4-fluorophenyl)-N-(1-methyl-6-phenyl-4H-[1,2,4]triazolo[4,3-a][1,4]benzodiazepin-4-yl)-2-propenamide

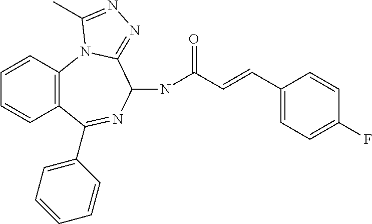

The title compound was prepared in similar fashion to Example 64 from (2E)-3-(4-fluorophenyl)prop-2-enoic acid (Aldrich), LC/MS: m/z 438 [M+H]⁺, Rt 3.1 min.

### Example 66

### N-[2-(methyloxy)phenyl]-N′-(1-methyl-6-phenyl-4H-[1,2,4]triazolo[4,3-a][1,4]benzodiazepin-4-yl)urea

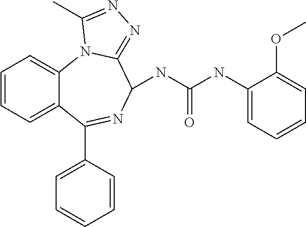

To a solution of 2-methoxyphenylisocyanate (8.7 mg, 0.058 mmol) in DCM (2 mL) was added one equivalent of Intermediate 28 in DCM (1 mL) and the reaction mixture was stirred overnight. The mixture was concentrated in vacuo to give a residue which after crystallisation from MeOH, gave the title compound as a white powder; HRMS (M+H)⁺ calculated for C₂₅H₂₃N₆O₂ (M+H)⁺ 439.1882; found 439.1888.

Examples 67 to 78 of formula (ID were prepared by methods analogous to that described for Example 66 using the starting materials indicated (see Table 9).

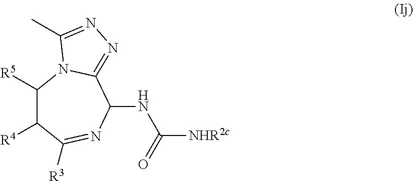

TABLE 9

| Ex | R³ | R⁴/R⁵ | R^{2c} | from Int | Physical data |
|---|---|---|---|---|---|
| 67 | phenyl | (R⁴)— | 4- | 1-iso- | HRMS |
| | | (CH)₄— | methoxy- | cyanato-4- | (M + H)⁺ |
| | | (R⁵) | phenyl | (methyloxy) | calculated |
| | | | | benzene | for |
| | | | | (Aldrich ) | C₂₅H₂₃N₆O₂ |
| | | | | | 439.1882 |
| | | | | | found |
| | | | | | 439.1888; Rt |
| | | | | | 2.27 |
| 68 | phenyl | (R⁴)— | 2- | 1-bromo-2- | APCl MS m/z |
| | | (CH)₄— | bromo- | isocyanato- | 486.62 |
| | | (R⁵) | phenyl | benzene | (M + H)⁺; |
| | | | | (Aldrich) | APCl |
| | | | | | MS m/z |
| | | | | | 485.85 |
| | | | | | (M − H)⁻; |
| | | | | | Rt 2.51 |
| 69 | phenyl | (R⁴)— | 4- | 1-bromo-4- | LC/MS : |
| | | (CH)₄— | bromo- | isocyanato- | APCl |
| | | (R⁵) | phenyl | benzene | m/z 486.66; |
| | | | | (Aldrich) | (M + H)⁺ m/z |
| | | | | | 484.72; |
| | | | | | (M − H)−; |
| | | | | | Rt 2.64 |
| 70 | phenyl | (R⁴)— | phenyl | isocyanato- | LC/MS: APCl |
| | | (CH)₄— | | benzene | m/z: 409.29; |
| | | (R⁵) | | (Aldrich) | (M + H)⁺Rt |
| | | | | | 2.35; |
| | | | | | m.p. > 260° C. |
| 71 | phenyl | (R⁴)— | phenyl- | (isocyanato- | LC/MS: APCl |
| | | (CH)₄— | methyl | methyl)- | m/z: 423.32; |
| | | (R⁵) | | benzene | (M + H)⁺; Rt |
| | | | | (Aldrich) | 2.22; |
| | | | | | m.p. > 260° C. |
| 72 | phenyl | (R⁴)— | phenethyl | (2- | HRMS |
| | | (CH)₄— | | isocyanato- | (M + H)⁺: |
| | | (R⁵) | | ethyl)- | calculated |
| | | | | benzene | for |
| | | | | (Aldrich) | C₂₆H₂₅N₆O |
| | | | | | 437.2090 |
| | | | | | found |
| | | | | | 437.2089; |
| | | | | | Rt 2.32 |
| 73 | phenyl | (R⁴)— | 3,4- | 5-iso- | HRMS |
| | | (CH)₄— | methylene- | cyanato-1,3- | (M + H)⁺ |
| | | (R⁵) | dioxy- | benzo- | calculated |
| | | | phenyl | dioxole | for |
| | | | | (Aldrich) | C₂₅H₂₁N₆O₃ |
| | | | | | 453.1675 |
| | | | | | found |
| | | | | | 453.1559; |
| | | | | | Rt 2.31 |
| 74 | phenyl | (R⁴)— | 4-nitro- | 1-iso | LC/MS: |
| | | (CH)₄— | phenyl | cyanato-4- | APCl m/z: |
| | | (R⁵) | | nitro- | 453.68 |
| | | | | benzene | (M + H)⁺; |
| | | | | (Aldrich) | m/z: |
| | | | | | 451.71 |
| | | | | | (M − H)⁻; |
| | | | | | Rt 2.52 |
| 75 | phenyl | (R⁴)— | 4-methoxy- | 1- | LC/MS |
| | | (CH)₄— | phenyl- | (isocyanato- | APCl |
| | | (R⁵) | methyl | methyl)- | m/z: |
| | | | | 4-(methyloxy) | 452.69 |
| | | | | benzene | (M + H)⁺; |
| | | | | (Aldrich) | Rt 2.22. |
| 76 | phenyl | (R⁴)— | cyclohexyl | Isocyanato- | LC/MS: |
| | | (CH)₄— | | cyclo- | APCl |
| | | (R⁵) | | hexane | m/z: |
| | | | | (Aldrich) | 415.67: |
| | | | | | (M + H)⁺; |
| | | | | | Rt 2.31 |
| | | | | | No NMR |
| 77 | phenyl | (R⁴)— | 2-methyl- | 1- | LC/MS: |
| | | (CH)₄— | phenyl- | (isocyanato- | APCl |
| | | (R⁵) | methyl | methyl)- | m/z: |
| | | | | 2-methyl- | 437.58: |
| | | | | benzene | (M + H)⁺; |
| | | | | (Aldrich) | Rt 2.33 |
| 78 | phenyl | (R⁴)— | 4-bromo- | 1-bromo-4- | HRMS |
| | | (CH)₄— | phenyl- | (isocyanato- | (M + H)⁺: |
| | | (R⁵) | methyl | methyl) | calculated |
| | | | | benzene | for |
| | | | | (Aldrich) | C₂₅H₂₂BrN₆O |
| | | | | | 501.1038 |
| | | | | | found |
| | | | | | 501.0988; |
| | | | | | Rt 2.55 |
| | | | | | No NMR |

### Example 79

### N-(8-iodo-1-methyl-6-phenyl-4H-[1,2,4]triazolo[4,3-a][1,4]benzodiazepin-4-yl)-N′-[4-(phenylcarbonyl)phenyl]urea

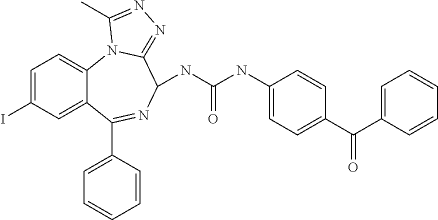

A solution of Intermediate 29 (100 mg, 0.24 mmol), Intermediate 88 (105 mg, 0.29 mmol, 1.2 equiv.) and TEA (1 mL) in THF was stirred at RT for 4 h. Then 0.5 equiv. of Intermediate 29 were added again and the mixture was stirred for 1 h before being concentrated, extracted with ethyl acetate/1 N NaOH, washed with water and brine, dried over Na₂SO₄, concentrated. The crude product was triturated twice in a hot solution of acetonitrile and filtered at this temperature to give the title compound as a cream solid; LC/MS: APCI (M+H)⁺ 639.01: (M−H)=636.96; Rt 2.77.

### Example 80

### N-{1-methyl-6-[4-(methyloxy)phenyl]-4H-[1,2,4]triazolo[4,3-a][1,4]benzo diazepin-4-yl}-N′-[4-(phenylcarbonyl)phenyl]urea

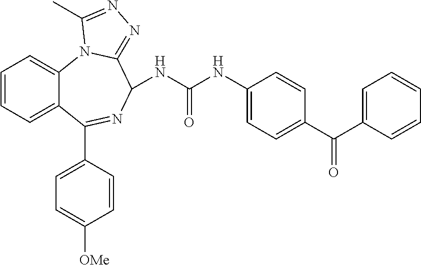

To a suspension of Intermediate 27 (140 mg, 0.44 mmol, 1 equiv.), Intermediate 88 (175 mg, 0.48 mmol, 1.1 equiv.) in DCM (2 mL) at 0° C., was added TEA (74 μL, 0.53 mmol, 1.2 equiv.). The reaction mixture was stirred at this temperature for 5 h30 before being quenched with 1N HCl. The organic layer was washed with NaOH 1 N, brine, dried over Na₂SO₄ and evaporated to dryness. The residue was recrystallized in acetonitrile, and washed twice with diethyl ether to give the title compound as a white solid; m.p. 225° C.; HRMS calculated for C₃₂H₂₇N₆O₃ (M+H)⁺ 543.2145 found 543.2108; Rt=2.82 min.

### Example 81

### N-(4-azidophenyl)-N′-(1-methyl-6-phenyl-4H-[1,2,4]triazolo[4,3-a][1,4]benzodiazepin-4-yl)thiourea

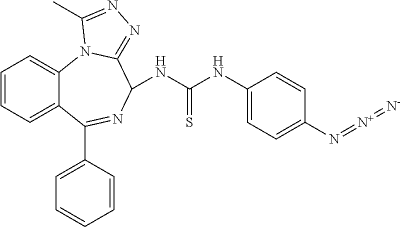

A solution of Intermediate 28 (50 mg, 0.173 mmol) and 4-azidophenylisothiocyanate (30.5 mg, 0.173 mmol; 1 equiv.) in dry DCM (6 mL) was stirred at RT overnight protected from light. The solid formed was filtered to give a cream solid. LC/MS (M+H)⁺ 466.12; (M−H) 464.10.

Examples 82 to 84 of formula (Id) were prepared by methods analogous to that described for Example 81 using the starting materials indicated (see Table 10).

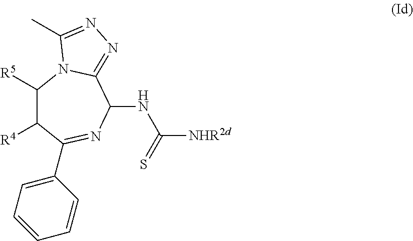

TABLE 10

| Ex. | R^{2d} | R⁴/R⁵ | From Int. | Physical data |
|---|---|---|---|---|
| 82 | 4-azidophenyl | (R⁴)— | 4-Azidophenyl- | LC/MS (M + H)⁺ |
| | | CH—C(I)— | isothiocyanate | 592.06; (M − H) |
| | | (CH)₂—(R⁵) | (Aldrich) and | 590.04 Rt = 2.90 |
| | | | Intermediate 29 | min |
| 83 | 4-(phenyl | (R⁴)—(CH)₄ | Benzophenone- | HRMS |
| | carbonyl)phenyl | —(R⁵) | 4- | calculated for |
| | | | isothiocyanate | C₃₁H₂₅N₆OS |
| | | | (Sigma) and | (M + H)⁺ |
| | | | Intermediate 28 | 529.1810; |
| | | | | found 529.1868 |
| 84 | 4-(phenyl | (R⁴)— | Benzophenone- | LC/MS (M + H)⁺ |
| | carbonyl)phenyl | CH—C(I)— | 4-isothiocyanate | 655.08; (M − H) |
| | | (CH)₂—(R⁵) | (Sigma) and | 653.04 Rt = |
| | | | Intermediate 29 | 3.09 min |

### Example 85

### 3-bromo-N-(1-methyl-6-phenyl-4H-benzo[f][1,2,4]triazolo[4,3-a][1,4]diazepin-4-yl)benzamide

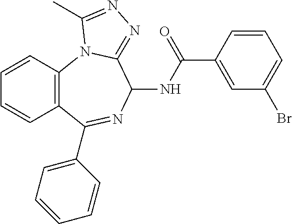

To a vial containing a solution of 1-methyl-6-phenyl-4H-[1,2,4]triazolo[4,3-a][1,4]benzodiazepin-4-amine (50 mg, 0.173 mmol), and 3-bromobenzoyl chloride (45.5 mg, 0.207 mmol), in DCM (1 mL) was added TEA (0.036 mL, 0.259 mmol). The mixture was stirred at RT overnight and concentrated to dryness. Purification of the residue by Agilent HPLC (20-100% MeCN/water+0.05% TFA) and concentration gave the title compound (22.8 mg, 0.048 mmol). LC/MS (M(⁷⁹Br)+H)⁺ 472, (M(⁸¹Br)+H)⁺ 474; RT 0.89 min.

### Example 86

### N-(1-methyl-6-phenyl-4H-benzo[f][1,2,4]triazolo[4,3-a][1,4]diazepin-4-yl)-2-naphthamide

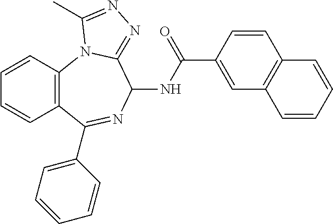

To a vial containing a solution of 1-methyl-6-phenyl-4H-[1,2,4]triazolo[4,3-a][1,4]benzodiazepin-4-amine (50 mg, 0.173 mmol), and 2-naphthalenecarbonyl chloride (39.5 mg, 0.207 mmol), in DCM (1 mL) was added TEA (0.036 mL, 0.259 mmol). The mixture was stirred at RT overnight and concentrated to dryness. Purification of the residue by Agilent HPLC (20-100% MeCN/water+0.05% TFA) and concentration gave the title compound (29.6 mg, 0.067 mmol). LC/MS (M+H)⁺ 444; RT 0.90 min.

Further Examples of the invention include:

| Ex- | |
|---|---|
| am- | |
| ple | |
| No. | Structure |
| 87 | 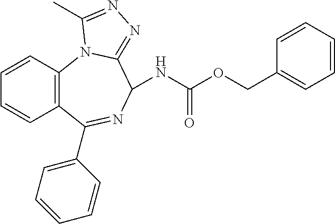 |
| 88 | 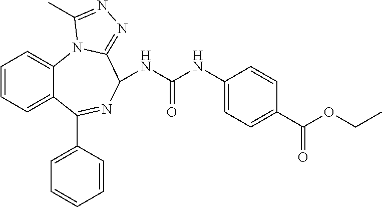 |
| 89 | 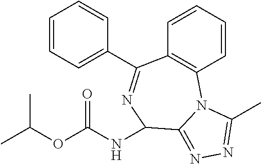 |
| 90 | 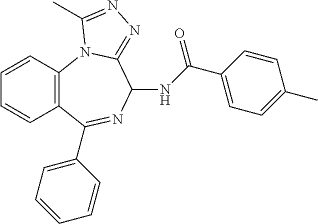 |
| 91 | 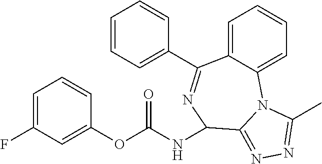 |
| 92 | 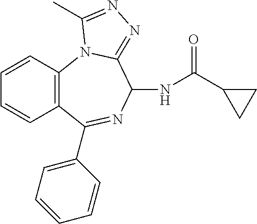 |
| 93 | 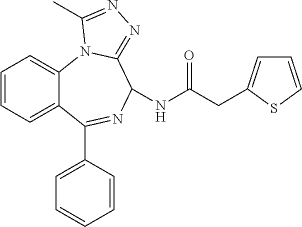 |
| 94 | 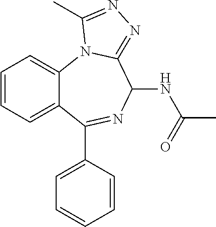 |
| 95 | 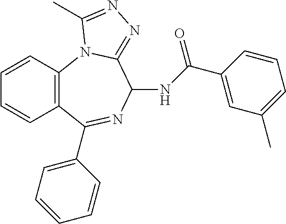 |
| 96 | 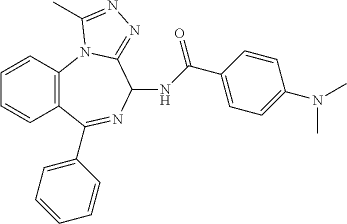 |
| 97 | 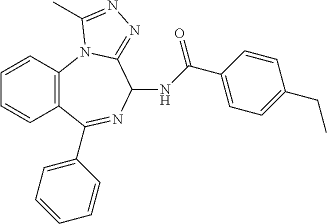 |
| 98 | 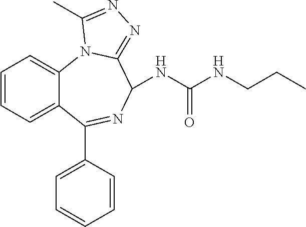 |
| 99 | 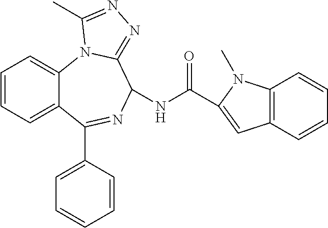 |
| 100 | 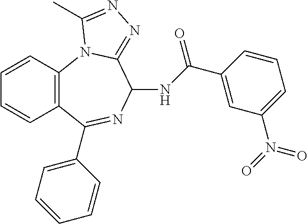 |
| 101 | 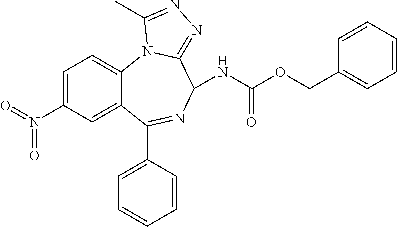 |
| 102 | 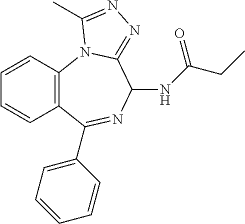 |
| 103 | 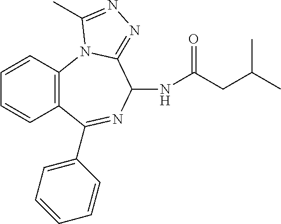 |
| 104 | 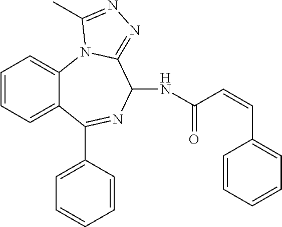 |
| 105 | 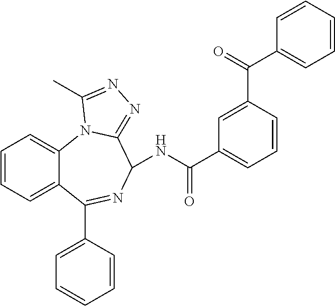 |
| 106 | 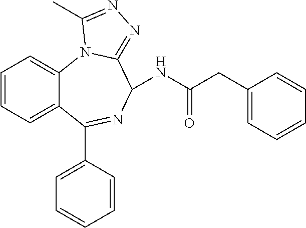 |
| 107 | 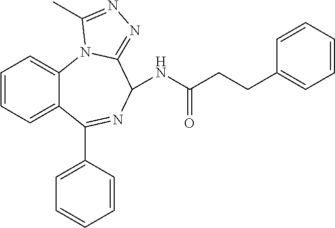 |
| 108 | 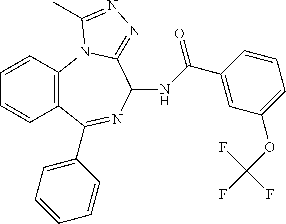 |
| 109 | 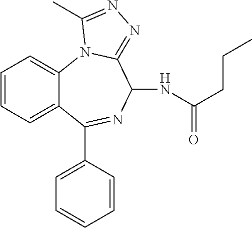 |
| 110 | 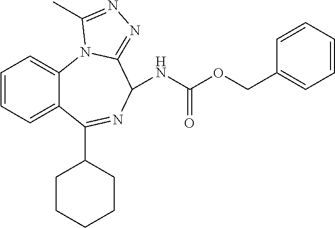 |
| 111 | 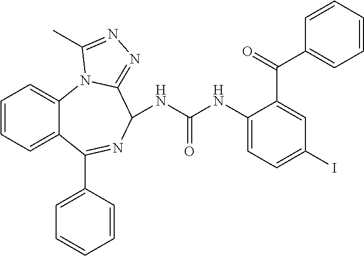 |
| 112 | 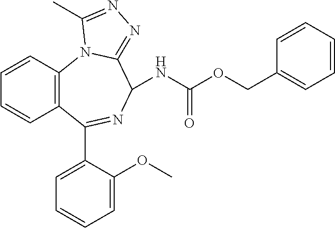 |
| 113 | 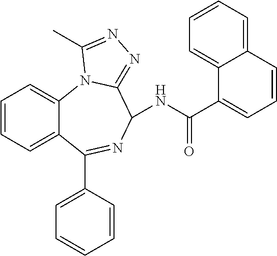 |
| 114 | 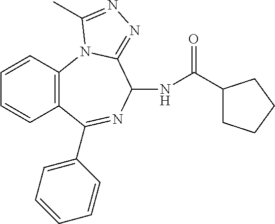 |
| 115 | 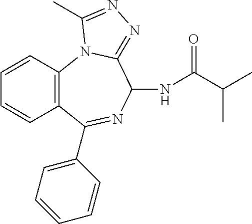 |
| 116 | 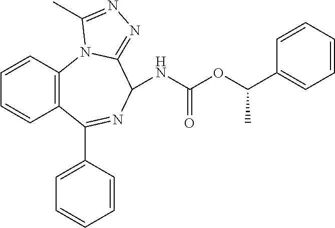 |
| 117 | 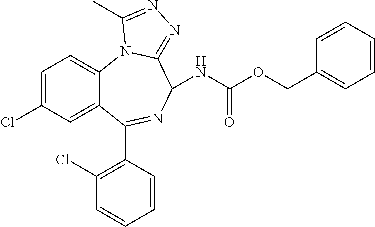 |
| 118 | 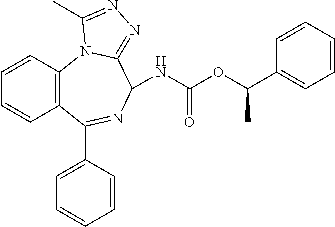 |
| 119 | 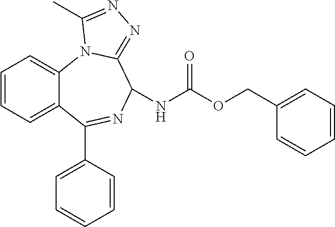 |
| 120 | 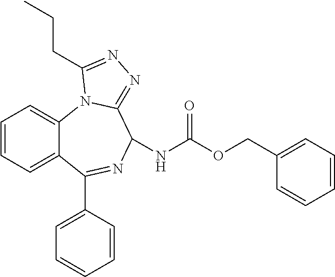 |
| 121 | 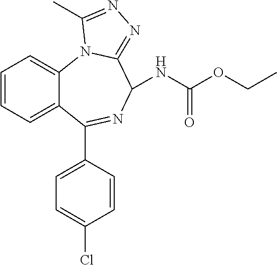 |
| 122 | 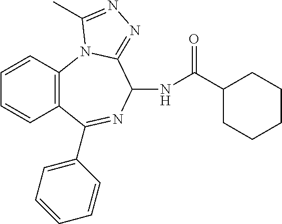 |
| 123 | 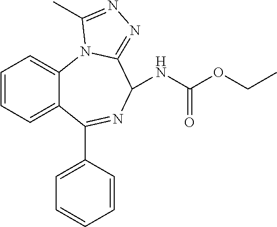 |
| 124 | 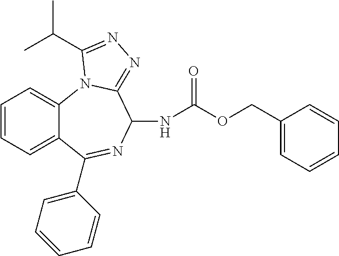 |
| 125 | 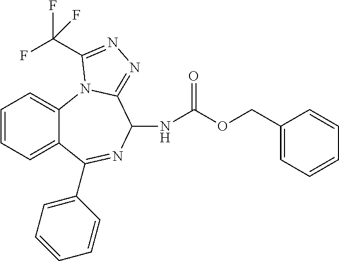 |
| 126 | 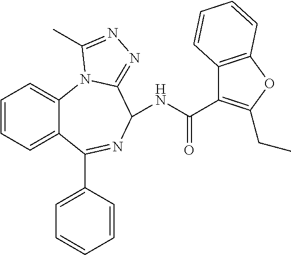 |
| 127 | 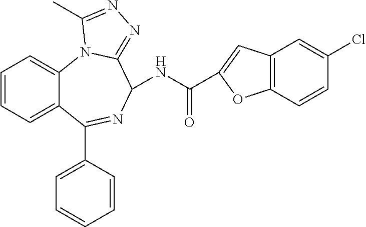 |
| 128 | 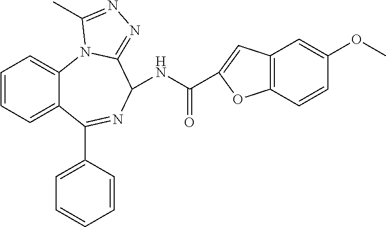 |
| 129 | 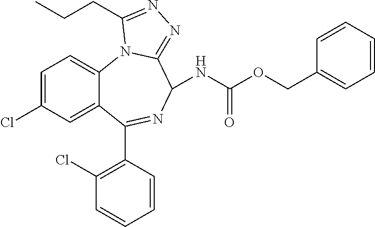 |
| 130 | 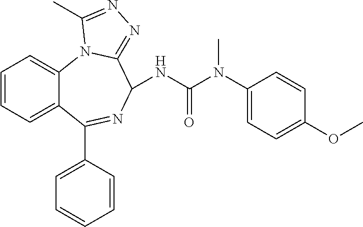 |
| 131 | 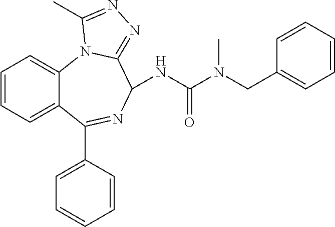 |
| 132 | 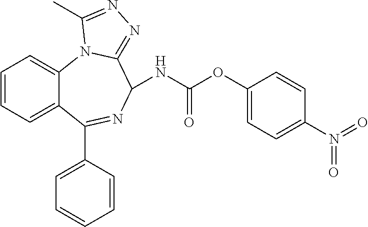 |
| 133 | 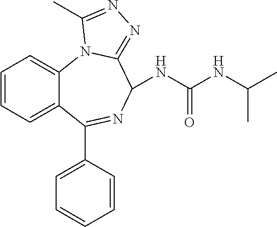 |
| 134 | 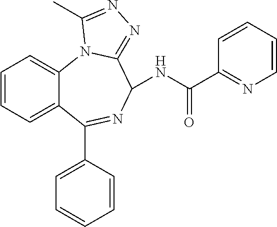 |
| 135 | 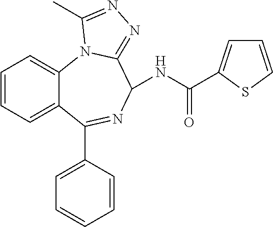 |
| 136 | 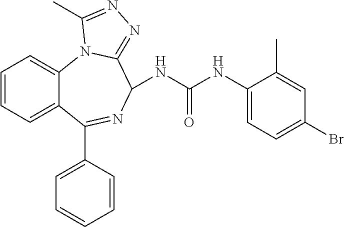 |
| 137 | 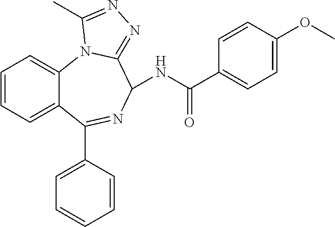 |
| 138 | 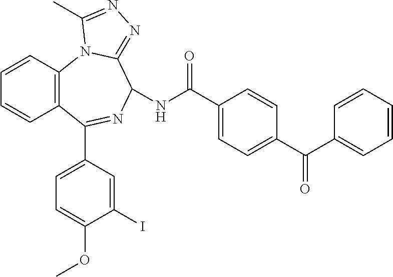 |
| 139 | 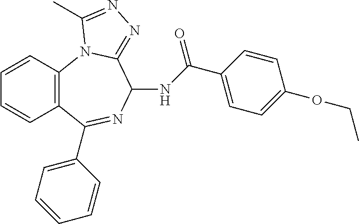 |
| 140 | 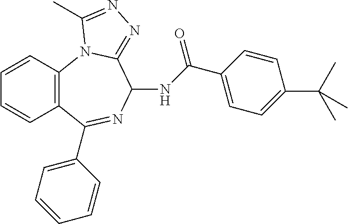 |
| 141 | 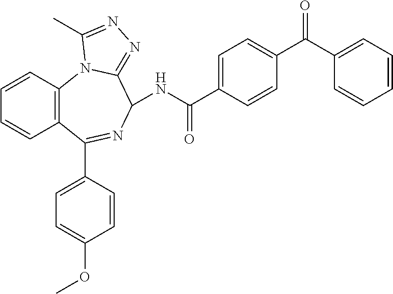 |
| 142 | 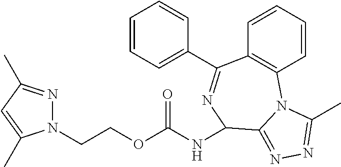 |
| 143 | 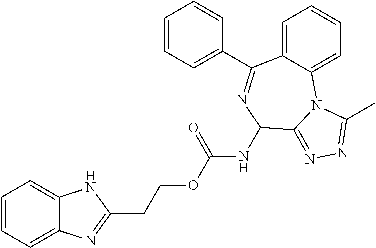 |
| 144 | 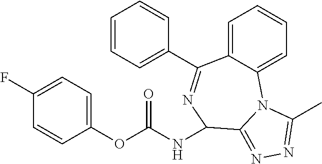 |
| 145 | 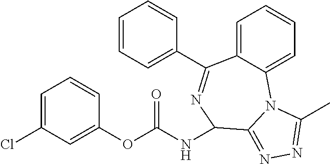 |
| 146 | 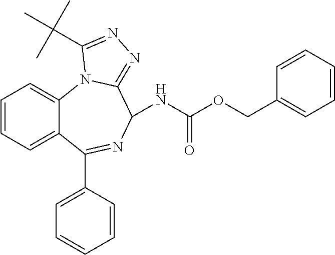 |
| 147 | 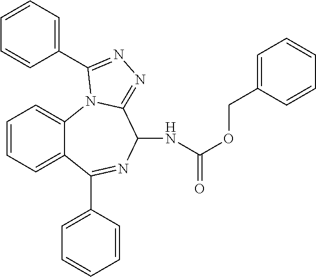 |
| 148 | 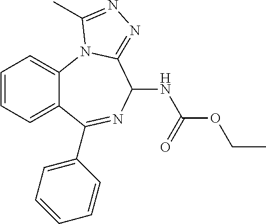 |
| 149 | 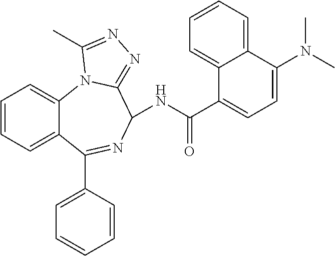 |
| 150 | 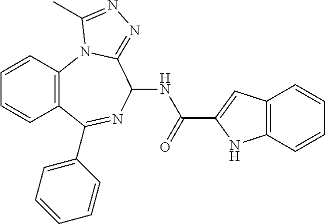 |

### Intermediates

### Intermediate 1: phenylmethyl [5-(4-fluorophenyl)-2-thioxo-2,3-dihydro-1H-1,4-benzodiazepin-3-yl]carbamate

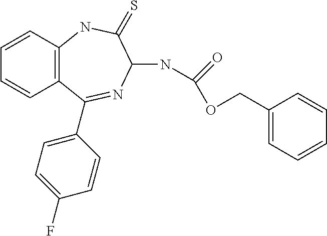

Lawesson's reagent (252 mg, 0.6 mmol, 0.6 equiv) was added to a suspension of Intermediate 37 (419 mg, 1.0 mmol) in toluene (5 mL) and the reaction mixture was heated to reflux under nitrogen for 4 h and then allowed to cool to RT. The resulting solid was filtered off, washed with toluene (40 mL) and then Et₂O (20 mL) to give the title compound (204 mg, 47%) as a cream solid; LC/MS: m/z 419 [M+H]⁺, Rt 3.6 min.

Intermediates 2 to 16 of formula (III) were prepared by methods analogous to that described for Intermediate 1 from the starting materials indicated (see Table 11).

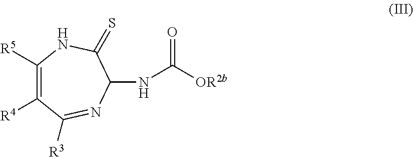

TABLE 11

| Int | R³ | R⁴/R⁵ | R^{2b} | From Int. | Physical data |
|---|---|---|---|---|---|
| 2 | 4-Cl- | (R⁴)—(CH)₄—(R⁵) | benzyl | 38 | LC/MS: m/z |
| | phenyl | | | | 436 [M + H]⁺, |
| | | | | | Rt 3.7 min. |
| 3 | 4-Cl- | (R⁴)—(CH)₄—(R⁵) | ethyl | 35 | LC/MS: m/z |
| | phenyl | | | | 374.30 [M + H]⁺, |
| | | | | | Rt 2.72 min |
| 4 | phenyl | (R⁴)—CH—C(OMe)—(CH)₂—(R⁵) | benzyl | 45 | LC/MS: m/z |
| | | | | | 432.09 [M + H]⁺, |
| | | | | | Rt 2.84 min |
| 5 | 4-MeO- | (R⁴)—(CH)₄—(R⁵) | benzyl | 39 | LC/MS: m/z |
| | phenyl | | | | 431 [M + H]⁺, |
| | | | | | Rt 3.5 min. |
| 6 | 4-tolyl | (R⁴)—(CH)₄—(R⁵) | benzyl | 40 | LC/MS: m/z |
| | | | | | 438 [M + H]⁺, |
| | | | | | Rt 3.16 min |
| 7 | 3-MeO- | (R⁴)—(CH)₄—(R⁵) | benzyl | 41 | LC/MS: m/z |
| | phenyl | | | | 432 [M + H]⁺, |
| | | | | | Rt 3.46 min. |
| 8 | phenyl | (R⁴)—(CH)₂—C(Me)—CH—(R⁵) | benzyl | 42 | LC/MS: m/z |
| | | | | | 416 [M + H]⁺, |
| | | | | | Rt 3.6 min. |
| 9 | phenyl | (R⁴)—CH—C(Cl)—(CH)₂—(R⁵) | benzyl | available from | LC/MS: m/z |
| | | | | Neosystem | 436 [M + H]⁺, |
| | | | | | Rt 3.7 min. |
| 10 | phenyl | (R⁴)—CH—CH—S—(R⁵) | benzyl | 43 | LC/MS: m/z |
| | | | | | 408 [M + H]⁺, |
| | | | | | Rt 3.5 min. |
| 11 | phenyl | (R⁴)—S—CH—CH—(R⁵) | benzyl | 44 | LC/MS: m/z |
| | | | | | 408 [M + H]⁺, |
| | | | | | Rt 3.4 min. |
| 12 | 2-F- | (R⁴)—CHC(Br)—(CH)₂—(R⁵) | benzyl | available from | LC/MS: m/z |
| | phenyl | | | Neosystem | 498.21 [M − H]⁺, |
| | | | | | Rt 3.07 min. |
| 13 | phenyl | 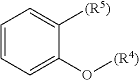 | benzyl | 47 | HRMS (M + H)⁺ calculated for C₂₅H₂₀N₃O₃S 442.1225 found 442.1074 |
| 14 | 4-Cl- Phenyl | 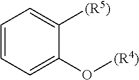 | ethyl | 48 | LC/MS: m/z 414 [M(³⁵Cl) + H]⁺, Rt 2.71 min |
| 15 | 4-OMe- | (R⁴)—(CH)₄—(R⁵) | benzyl | 49 | HRMS (M + H)⁺ |
| | phenyl | | | | calculated for |
| | | | | | C₂₄H₂₂N₃O₃S 432.1382 |
| | | | | | found 432.1431. |
| 16 | 4-Cl- | (R⁴)—CH—C(OMe)—(CH)₂—(R⁵) | benzyl | 46 | LC/MS: m/z |
| | Phenyl | | | | 465.97 [M + H]+, |
| | | | | | Rt 2.87 min |

### Intermediate 17: phenylmethyl [6-(2-fluorophenyl)-1-methyl-8-(tributylstannanyl)-4H-[1,2,4]triazolo[4,3-a][1,4]benzodiazepin-4-yl]carbamate

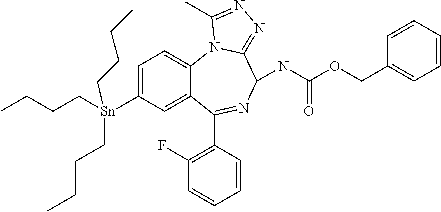

To a solution of Example 17 (600 mg, 1.15 mmol) in dry toluene (15 mL), was added (Bu₃Sn)₂ (1 g, 1.73 mmol, 1.5 equiv.) and Pd(PPh₃)₄ (cat). The reaction was carried under microwaves (P=100 W, T=200° C., 10 min Pmax=10 bars) and purified by chromatography (DCM/MeOH 98/2); APCI (M+H)=732.2, Rt=4.29 min.

### Intermediate 18: phenylmethyl {7-bromo-5-(2-fluorophenyl)-2-[(1Z)-hydrazino]-3H-1,4-benzodiazepin-3-yl}carbamate

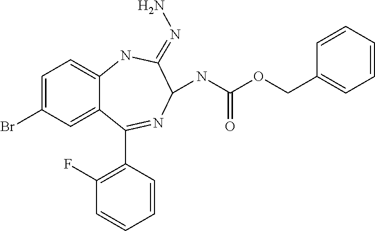

To a solution of phenylmethyl [7-bromo-5-(2-fluorophenyl)-2-oxo-2,3-dihydro-1H-1,4-benzodiazepin-3-yl]carbamate (Neosystem; 950 mg, 1.9 mmol) in MeOH (25 mL) was added hydrazine hydrate (925 μl, 19 mmol) and the mixture was stirred at room temperature for 2 hours. The mixture was concentrated to half volume and extracted with EtOAc (2×50 mL). The extracts were dried (sodium sulfate) and concentration under reduced pressure to give the title compound as a crude product which was used in the next step without purification; LC/MS APCI m/z: 496.23 and 498.23 (Br isotopes), Rt: 2.83 min.

Intermediates 19 to 25 of formula (II) were prepared by methods analogous to that described for Intermediate 18 using the starting materials indicated (see Table 12).

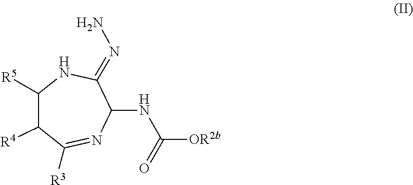

TABLE 12

| Int | R³ | R^{2b} | R⁴/R⁵ | From Int. | Physical data |
|---|---|---|---|---|---|
| 19 | phenyl | benzyl | 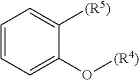 | 13 | HRMS calculated for C₂₅H₂₁N₅O₃ (M + H)⁺ 440.1722 found 440.1628. |
| 20 | 4-Cl- phenyl | ethyl | 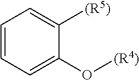 | 14 | LC/MS: m/z 412 [M(³⁵Cl) + H]⁺, Rt 2.64 min |
| 21 | 4-MeO- | benzyl | (R⁴)—(CH)₄—(R⁵) | 5 | HRMS (M + H)⁺ calculated |
| | phenyl | | | | for C₂₄H₂₄N₅O₃ 430.1879 |
| | | | | | found 430.1891. |
| 22 | 4-F- | benzyl | (R⁴)—(CH)₄—(R⁵) | 1 | ¹H NMR (300 MHz, |
| | phenyl | | | | DMSO d6) δ ppm: 7.72 |
| | | | | | (d, 1H, J = 8.5 Hz), 7.56- |
| | | | | | 7.46 (m, 4H), 7.44-7.17 |
| | | | | | (m, 10H), 7.12 (m, 1H), |
| | | | | | 7.04 (m, 1H), 5.37 (d, |
| | | | | | 1H, J = 8.3 Hz), 5.06 (s, 2H). |
| 23 | 4-F- | ethyl | (R⁴)—(CH)₄—(R⁵) | 30 | LC/MS: m/z 356 [M + H]⁺, |
| | phenyl | | | | Rt 2.11 min. |
| 24 | phenyl | benzyl | (R⁴)—CH—C(I)—(CH)₂—(R⁵) | 31 | LC/MS APCI m/z: |
| | | | | | 526.28 [M + H]⁺ and |
| | | | | | 524.29 [M + H]⁻, Rt: 2.97 min. |
| 25 | phenyl | t-butyl | (R⁴)—CH—C(I)—(CH)₂—(R⁵) | 32 | LC/MS: 492.0684 |
| | | | | | [M + H]+, Rt 3.10 min. |

### Intermediate 26: 1-ethyl-6-(4-fluorophenyl)-4H-[1,2,4]triazolo[4,3-a][1,4]benzodiazepin-4-amine

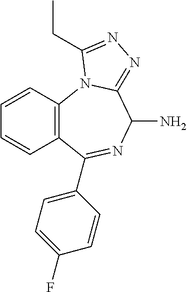

To a suspension of Example 22 (100 mg, 0.22 mmol) in MeOH (1 mL) at RT was added Pd on carbon (100 mg, 10%, 94 μmol, 0.4 equiv.) and 1,4-cyclohexadiene (0.11 mL, 1.1 mmol, equiv.). The reaction mixture was stirred for 1.5 h before being filtered on Celite and concentrated to give the title compound (60 mg, 85%) as a colorless oil; R_{f}=0.54 (DCM/MeOH:90/10); HRMS (M+H)⁺ calculated for C₁₈H₁₇FN₅ 322.1468 found 322.1393.

### Intermediate 27: 1-methyl-6-[4-(methyloxy)phenyl]-4H-[1,2,4]triazolo[4,3-a][1,4]benzo diazepin-4-amine

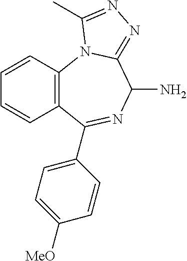

To a suspension of Example 21 (0.85 g, 1.9 mmol) in MeOH (10 mL) at RT was added Pd on carbon (0.85 g, 10%, 0.8 mmol, 0.4 equiv.) and 1,4-cyclohexadiene (0.95 mL, 9.4 mmol, 5 equiv.). The reaction mixture was stirred for 2 h before being filtered on Celite and concentrated to give the title compound (0.3 g, 85%) as a white solid; R_{f}=0.24 (DCM/MeOH:95/5); LC/MS: m/z 303 [M−NH₂]⁺, Rt 1.61 min.

### Intermediate 28: 1-methyl-6-phenyl-4H-[1,2,4]triazolo[4,3-a][1,4]benzodiazepin-4-amine

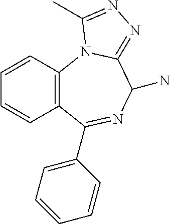

To a solution of phenylmethyl (1-methyl-6-phenyl-4H-[1,2,4]triazolo[4,3-a][1,4]benzodiazepin-4-yl)carbamate [see J. Med. Chem., (1988), 31(1), 176-181)] (4 g, 9.5 mmol) in methanol (100 mL) under nitrogen was added palladium/carbon catalyst (4 g, 10%) followed by 1,4 cyclohexadiene (6 mL) and the reaction mixture was stirred at RT for 4 hours. The mixture was filtered through Celite and the filtrate was evaporated under reduced pressure to afford the title compound which was used directly in the next step without further purification; ¹H NMR (300 MHz, CDCl₃) δ ppm: 7.6-7.2 (m, 9H), 4.9 (br s, 1H), 2.55 (s, 3H); [APCI MS] m/z 273.2 (MH⁺—NH₃).

### Intermediate 29: 8-iodo-1-methyl-6-phenyl-4H-[1,2,4]triazolo[4,3-a][1,4]benzodiazepin-4-amine

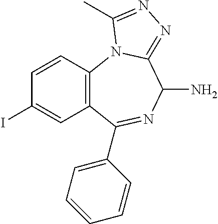

A solution of Example 25 (550 mg, 1.06 mmol) in DCM/TFA (8/2) was refluxed for 1 h. The resulting mixture was basified with NaOH 1N, extracted with DCM, dried over Na₂SO₄, concentrated and triturated in diethyl ether to give a white powder; LC/MS: 416.0374 [M+H]⁺, Rt 2.34 min.

### Intermediate 30: ethyl [5-(4-fluorophenyl)-2-thioxo-2,3-dihydro-1H-1,4-benzodiazepin-3-yl]carbamate

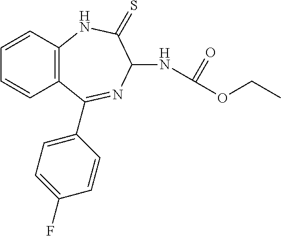

Lawesson's reagent (1.6 g, 4.0 mmol, 0.6 equiv.) was added to a suspension Intermediate 50 (2.3 g, 9.7 mmol) in toluene (24 mL). The reaction mixture was heated to reflux under nitrogen for 2 h before being concentrated under reduced pressure. Purification by flash-chromatography (DCM/MeOH:95/5) gave the title compound (2.31 g, 96%) as a yellow solid; R_{f}=0.44 (DCM/MeOH:98/2); LC/MS: m/z 358 [M+H]⁺, Rt 2.48 min; HRMS calculated for C₁₈H₁₇FN₃O₂S (M+H)⁺ 358.1025 found 358.0956.

### Intermediate 31: phenylmethyl (7-iodo-5-phenyl-2-thioxo-2,3-dihydro-1H-1,4-benzo diazepin-3-yl)carbamate

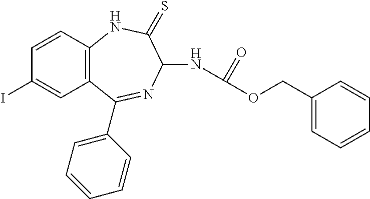

A solution of Intermediate 51 (5 g, 9.8 mmol) in toluene (100 mL) and Lawesson's reagent (4.3 g, 10.8 mmol, 1.1 equiv.) was heated at 100° C. for 3 h. The product was extracted with EtOAc/H₂O, dried over Na₂SO₄, concentrated and purified by flash chromatography to give the title compound as a cream foam; LC/MS: m/z 528.16 [M+H]⁺, 526.16 [M−H], Rt 3.13 min.

### Intermediate 32: 1,1-dimethylethyl (7-iodo-5-phenyl-2-thioxo-2,3-dihydro-1H-1,4-benzodiazepin-3-yl)carbamate

The title compound was prepared in similar fashion to Intermediate 31 from Intermediate 33; LC/MS: 437.9585 [M+H-tBu]⁺, Rt 3.38 min.

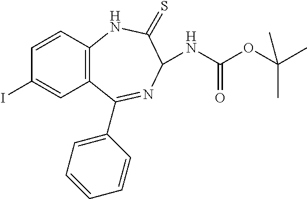

### Intermediate 33: 1,1-dimethylethyl (7-iodo-2-oxo-5-phenyl-2,3-dihydro-1H-1,4-benzo diazepin-3-yl)carbamate

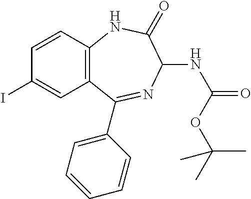

To a solution of Intermediate 34 (2.5 g, 6.6 mmol) and TEA (1.4 mL, 9.95 mmol, 1.5 equiv.) in dry THF was added dropwise a solution of BOC₂O (1.44 g, 6.6 mmol, 1 equiv.) in THF. The reaction mixture was stirred at RT for 1 h. The resulting mixture was extracted with EtOAc/water, the organic layer was washed with NaHCO₃sat., brine, dried and concentrated; HRMS calculated for C₁₆H₁₃IN₃O₃ (M−H-tBu)⁺ 422.0002 found 421.9796.

### Intermediate 34: 3-amino-7-iodo-5-phenyl-1,3-dihydro-2H-1,4-benzodiazepin-2-one

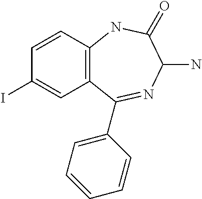

To a solution HBr/AcOH 30% (50 mL) was carefully added Intermediate 51 (5 g, 0.98 mmol). The reaction mixture was stirred at 80° C. for 1 h. After return to RT, the precipitate was filtered, washed twice with diethyl ether; then it was dissolved in EtOAc/H₂O, basified with NaOH 1N. The organic layer containing an insoluble was diluted in ethanol and evaporated; HRMS calculated for C₁₅H₁₃1N₃O (M+H)⁺ 378.0103 found 378.9972.

Intermediate 35: ethyl [5-(4-chlorophenyl)-2-oxo-2,3-dihydro-1H-1,4-benzodiazepin-3-yl]-carbamate

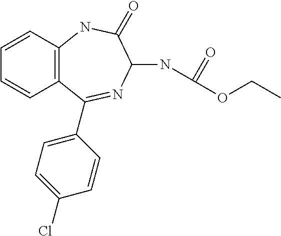

To a suspension of Intermediate 36 (5.34 g, 18.7 mmol) in THF (100 mL), was added TEA (2.87 mL, 20.57 mmol, 1.1 equiv.), and was cooled to 0° C. Then, a solution of ethylchloroformate (1.97 mL, 20.57 mmol, 1.1 equiv.) in THF (10 mL) was added dropwise to the reaction mixture. After stirring 2 h, the reaction mixture was evaporated, diluted in DCM, washed with water, dried and evaporated to dryness. The residue was purified by flash chromatography (DCM/MeOH 98/2) to give the title compound as a white solid; LC/MS: m/z 358.13 [M+H]+, Rt 2.42 min.

### Intermediate 36: 3-amino-5-(4-chlorophenyl)-1,3-dihydro-2H-1,4-benzodiazepin-2-one

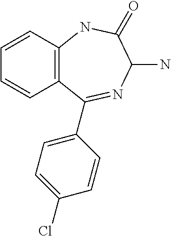

To a suspension of Intermediate 38 (7.5 g, 17.86 mmol) in AcOH (35 mL), was added HBr/AcOH 37% (35 mL, 178.6 mmol, 10 equiv.) and heated at 80° C. for 1 h. After allowing the resulting mixture to cool to RT, a yellow solid was formed, which was filtered and washed with diisopropyl ether. The solid was then stirred for 1 h in a mixture of DCM (250 mL) and NaOH 1N (250 mL). The solid was filtered and washed with water. The organic layer obtained was washed with water, dried, and evaporated to dryness; LC/MS: m/z 286.04 [M+H]+, Rt 1.87 min

### Intermediate 37: phenylmethyl [5-(4-fluorophenyl)-2-oxo-2,3-dihydro-1H-1,4-benzodiazepin-3-yl]carbamate

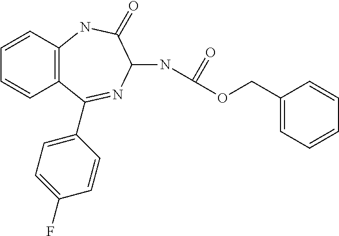

Ammonium acetate (470 mg, 6.1 mmol, 5 equiv.) was added to a solution of Intermediate 52 (515 mg, 1.22 mmol) in glacial acetic acid (10 mL) and stirred at RT for 24 h. The solvent was evaporated and the residue co-evaporated with toluene (2×20 mL). The mixture was basified with 2N NaOH (ca. 20 mL) and extracted with EtOAc (3×50 mL) and dried (Na₂SO₄) to give the title compound; LC/MS: m/z 404 [M+H]+, Rt 3.25 min; m.p. >260° C.;

HRMS calculated for C₂₅H₂₀N₃O₄ (M+H)⁺ 426.1454 found 426.1436.

Intermediates 38 to 46 of formula (V) were prepared by methods analogous to that described for Intermediate 37 from the starting materials indicated (see Table 13).

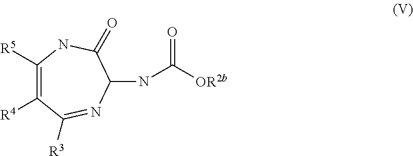

TABLE 13

| Int | R³ | R⁴/R⁵ | R^{2b} | From Int. | Physical data |
|---|---|---|---|---|---|
| 38 | 4-Cl—phenyl | (R⁴)—(CH)₄—(R⁵) | benzyl | 53 | LC/MS: m/z 420 |
| | | | | | [M + H]⁺, Rt |
| | | | | | 3.41 min. |
| 39 | 4-MeO—phenyl | (R⁴)—(CH)₄—(R⁵) | benzyl | 54 | LC/MS: m/z 416 |
| | | | | | [M + H]⁺, Rt |
| | | | | | 3.19 min. |
| 40 | 4-tolyl | (R⁴)—(CH)₄—(R⁵) | benzyl | 55 | LC/MS: m/z 400 |
| | | | | | [M + H]⁺, Rt |
| | | | | | 3.32 min. |
| 41 | 3-MeO—phenyl | (R⁴)—(CH)₄—(R⁵) | benzyl | 56 | LC/MS: m/z 416 |
| | | | | | [M + H]⁺, Rt |
| | | | | | 3.20 min. |
| 42 | phenyl | (R⁴)—(CH)₂—C(Me)—CH—(R⁵) | benzyl | 57 | LC/MS: m/z 400 |
| | | | | | [M + H]⁺, Rt |
| | | | | | 3.30 min. |
| 43 | phenyl | (R⁴)—CH—CH—S—(R⁵) | benzyl | 58 | LC/MS: m/z 392 |
| | | | | | [M + H]⁺, Rt |
| | | | | | 3.15 min. |
| 44 | phenyl | (R⁴)—S—CH—CH—(R⁵) | benzyl | 59 | LC/MS: m/z 392 |
| | | | | | [M + H]⁺, Rt |
| | | | | | 3.11 min. |
| 45 | phenyl | R⁴)—CH—C(OMe)—(CH)₂—(R⁵) | benzyl | 60 | LC/MS: m/z |
| | | | | | 416.09 [M + H]⁺, |
| | | | | | Rt 2.57 min. |
| 46 | 4-Cl—phenyl | R⁴)— CH—C(OMe)—(CH)₂—(R⁵) | benzyl | 61 | LC/MS: m/z |
| | | | | | 449.98 [M + H]⁺, |
| | | | | | Rt 2.81 min. |

### Intermediate 47: phenylmethyl (2-oxo-5-phenyl-2,3-dihydro-1H-[1]benzofuro[3,2-e][1,4]diazepin-3-yl)carbamate

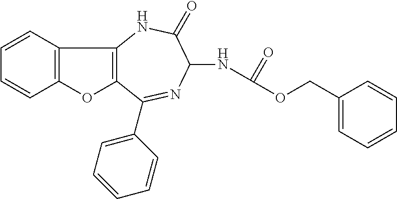

7N ammonia in MeOH (50 mL) was added to Intermediate 75 (assumed 21.1 mmol) at RT under nitrogen and the mixture was stirred at RT for 4 h. Acetic acid (20 mL, 0.35 mol, 17 equiv.) was then added dropwise. MeOH (50 mL) was then added and the reaction mixture was stirred for 2 days. The mixture was evaporated to dryness under reduced pressure. The solid was suspended in toluene (50 mL) and concentrated again (this procedure is repeated twice) in order to remove excess of AcOH. The resulting solid was then diluted with DCM (100 ml) and washed with water (1×50 mL) and saturated sodium bicarbonate solution (50 mL) before being concentrated under reduced pressure. Purification by flash-chromatography (DCM 100% to DCM/MeOH:95/5) gave the title compound as a yellow foam (1.9 g, 22%); R_{f}=0.50 (DCM/MeOH:95/5); m.p. >260° C.; HRMS calculated for C₂₅H₂₀N₃O₄ (M+H)⁺ 426.1454 found 426.1436.

Intermediate 48: ethyl [5-(4-chlorophenyl)-2-oxo-2,3-dihydro-1H-[1]benzofuro[3,2-e][1,4]diazepin-3-yl]carbamate

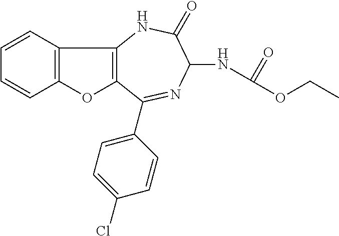

7N ammonia in MeOH (15 mL) was added to Intermediate 76 (assumed 73.6 mmol) at RT under nitrogen and the mixture was stirred at RT for 3 h before AcOH (90 mL, 1.6 mol, 17 equiv.) was added dropwise. MeOH (50 mL) was then added and the reaction mixture was stirred for 2 days before being filtered. The solid was washed with toluene (50 mL) and was triturated in hot CH₃CN before being filtered again to give the title compound as a green-yellow solid (11.4 g, 39%); R_{f}=0.20 (DCM/MeOH:95/5); m.p. >260° C.; LC/MS: m/z 398 [M(³⁵Cl)+H]⁺, Rt 2.64 min.

### Intermediate 49: phenylmethyl {5-[4-(methyloxy)phenyl]-2-oxo-2,3-dihydro-1H-1,4-benzodiazepin-3-yl}carbamate

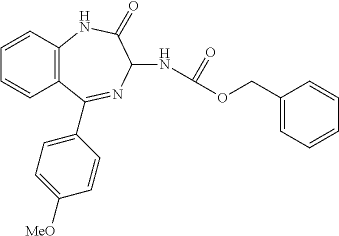

Ammonium acetate (22.4 g, 0.29 mol, 5 equiv.) was added to a solution of Intermediate 66 (58.1 mmol) in glacial acetic acid (400 mL) at RT and the reaction mixture was left to stir at RT for 15 h. The solvent was evaporated and the residue co-evaporated with toluene (2×200 mL). The resulting solid was filtered off and washed with toluene (2×50 mL) and the filtrate was washed with saturated aqueous NaHCO₃ (3×75 mL), dried over Na₂SO₄ and evaporated. Purification by flash-chromatography (DCM/MeOH:98/2) afforded the title compound (16.2 g, 67% over 3 steps) as a pale yellow solid; R_{f}=0.45 (DCM/MeOH:95/5); ¹H NMR (300 MHz, CDCl₃) δ ppm: 7.55-7.07 (m, 13H), 6.86 (d, 2H, J=8.6 Hz), 5.38 (d, 1H, J=8.3 Hz), 5.20 (s, 2H), 3.79 (s, 3H); HRMS (M+H)⁺ calculated for C₂₄H₂₂N₃O₄ 416.1610 found 416.1544.

### Intermediate 50: ethyl [5-(4-fluorophenyl)-2-oxo-2,3-dihydro-1H-1,4-benzodiazepin-3-yl]-carbamate

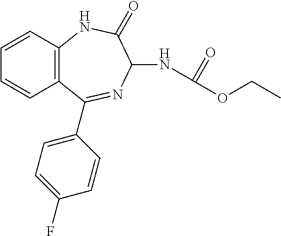

7N ammonia in MeOH (50 mL) was added to Intermediate 77 (assumed 50.2 mmol) at RT under nitrogen and the mixture was stirred for 2.5 h before AcOH (30 mL, 0.52 mol, 10 equiv.) was added dropwise. The reaction mixture was stirred for 2 days before being concentrated under reduced pressure to dryness. The resulting solid was suspended in DCM (100 mL) and washed with saturated sodium bicarbonate solution (150 mL) and brine (100 mL), dried over Na₂SO₄ and the solvent removed in vacuo. Purification by flash-chromatography (DCM/MeOH:95/5) gave the title compound as a white solid (2.3 g, 13% over 3 steps); R_{f}=0.22 (DCM/MeOH:95/5); ¹H NMR (300 MHz, DMSO d6) δ ppm: 10.86 (br s, 1H), 8.18 (d, 1H, J=8.5 Hz), 7.64 (m, 1H), 7.57-7.49 (m, 2H), 7.36-7.21 (m, 5H), 5.00 (d, 1H, J=8.5 Hz), 4.02 (q, 2H, J=7.1 Hz), 1.19 (t, 3H, J=7.1 Hz); LC/MS: m/z 342 [M+H]⁺, Rt 2.18 min; HRMS (M+H)⁺ calculated for C₁₈H₁₇FN₃O₃ 342.1254 found 342.1163.

### Intermediate 51: phenylmethyl (7-iodo-2-oxo-5-phenyl-2,3-dihydro-1H-1,4-benzodiazepin-3-yl)carbamate

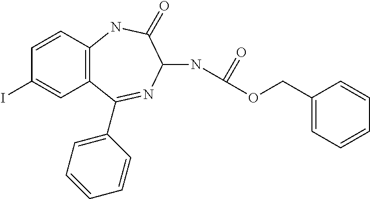

Ammonium acetate (30 g) was added to a solution of Intermediate 63 (4.5 g, 8.8 mmol) in glacial acetic acid (200 mL) and stirred at RT for 3 h. The precipitate formed was filtered, washed three times with water, and twice with diethyl ether to give the title compound as a white powder; LC/MS: m/z 512.17 [M+H]+, Rt=2.91 min.

### Intermediate 52: phenylmethyl [1-amino-2-({2-[(4-fluorophenyl)carbonyl]phenyl}amino)-2-oxoethyl]carbamate

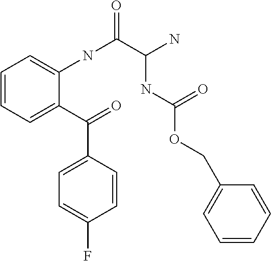

7N ammonia in MeOH (25 mL) was added to Intermediate 64 (633 mg, 1.2 mmol) at RT under nitrogen and the mixture was stirred at RT for 1 h. The reaction mixture was diluted with EtOAc (100 mL) and washed with 1N NaOH solution (2×50 mL). The aqueous layer was extracted with EtOAc (50 mL), and the combined organics were dried (Na₂SO₄) and evaporated to give the title compound as a yellow foam (515 mg, 100%); LC/MS: m/z 421 [M+H]⁺, Rt 2.7 min.

Intermediates 53 to 62 of general formula (VIa) were prepared by methods analogous to that described for Intermediate 52 using the starting materials indicated (see Table 14).

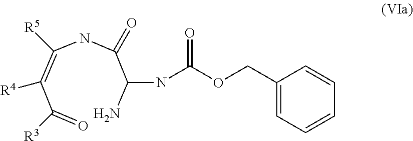

TABLE 14

| Int | R³ | R⁴/R⁵ | from Int. | Physical data |
|---|---|---|---|---|
| 53 | 4-Cl—phenyl | (R⁴)—(CH)₄—(R⁵) | 65 | LC/MS: m/z 436 [M − H]⁻, |
| | | | | Rt 2.83 min |
| 54 | 4-MeO—phenyl | (R⁴)—(CH)₄—(R⁵) | 66 | LC/MS: m/z 432 [M + H]⁺, |
| | | | | Rt 2.74 min. |
| 55 | 4-tolyl | (R⁴)—(CH)₄—(R⁵) | 67 | LC/MS: m/z 417 [M − H]⁻, |
| | | | | Rt 2.75 min |
| 56 | 3-MeO—phenyl | (R⁴)—(CH)₄—(R⁵) | 68 | LC/MS: m/z 432 [M − H]⁻, |
| | | | | Rt 2.68 min. |
| 57 | phenyl | (R⁴)—(CH)₂—C(Me)—CH—(R⁵) | 69 | LC/MS: m/z 416 [M − H]⁻, |
| | | | | Rt 2.85 min. |
| 58 | phenyl | (R⁴)—CH—CH—S—(R⁵) | 70 | LC/MS: m/z 408 [M − H]⁻, |
| | | | | Rt 2.80 min. |
| 59 | phenyl | (R⁴)—S—CH—CH—(R⁵) | 71 | LC/MS: m/z 408 [M − H]⁻, |
| | | | | Rt 2.68 min. |
| 60 | phenyl | R⁴)—CH—C(OMe)—(CH)₂—(R⁵) | 72 | LC/MS: m/z 433 [M − H]⁻, |
| | | | | Rt 2.53 min. |
| 61 | 4-Cl—phenyl | R⁴)—CH—C(OMe)—(CH)₂—(R⁵) | 73 | LC/MS: m/z 448.089 (468- |
| | | | | 20), Rt 3.16 min. |
| 62 | 4-OMe—phenyl | (R⁴)—(CH)₄—(R⁵) | 74 | HRMS (M + H)⁺ calculated |
| | | | | for C₂₄H₂₄N₃O₅ 434.1716 |
| | | | | found 434.1679. |

### Intermediate 63: phenylmethyl (1-amino-2-{[4-iodo-2-(phenylcarbonyl)phenyl]amino}-2-oxoethyl)carbamate

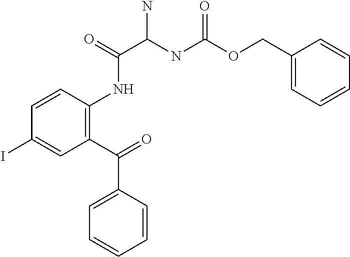

To a solution of Intermediate 82 (30 g, 92.8 mmol) in anhydrous THF (500 mL) at 0° C., was added dropwise oxalyle chloride (55 mL, 111.5 mmol, 1.2 equiv.) followed by DMF (5 mL). After stirring at 0° C. for 3 h, a solution of NMM (12.3 mL, 111.5 mmol, 1.2 equiv.) and 1H-1,2λ⁵,3-benzotriazol-2-yl({[(phenylmethyl)oxy]carbonyl}amino)acetic acid (30.2 mg, 92.8 mmol, 1 equiv.) in THF was added. The reaction mixture was stirred at 0° C. for 1 h and overnight at RT. The resulting mixture was hydrolyzed with water (200 mL), then the organic layer was extracted with ethyl acetate, washed with brine, dried and concentrated. The residue was partially dissolved in MeOH/NH₃ 7N (300 mL) and stirred at 0° C. for 1 h. The solid was filtered, washed with diethyl ether and dried to give the title compound.

### Intermediate 64: phenyl methyl [1-(1H-1,2,3-benzotriazol-1-yl)-2-({2-[(4-fluorophenyl)carbonyl]phenyl}amino)-2-oxoethyl]carbamate

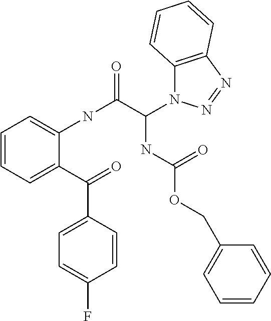

(2-Aminophenyl)(4-fluorophenyl)methanone (prepared according to WO00/05195) (440 mg, 2.0 mmol) and 1H-1,2,3-benzotriazol-1-yl{[(benzyloxy)carbonyl]amino}acetic acid ylcarbamate (see J. Org. Chem., (1990), 55, 2206) (1.0 g, 3.8 mmol 1.5 equiv.) in dry DCM (30 mL) were cooled to 0° C. under nitrogen. EDC (3.07 mmol,) and 4-DMAP (30 mg) were added, followed by N,N-diisopropylethylamine (534 μl, 1.5 equiv) after 10 min to aid solution. The resulting solution was allowed to warm to RT and stirred for 1 h. The reaction was washed with 8% sodium bicarbonate solution (2×40 mL) and brine (2×40 mL) and dried (aqueous extraction cartridge). The compound was purified using a 10 g Si SPE cartridge, eluting with DCM to DCM/EtOAc (4:1) to give the title compound as a yellow foam (633 mg, 59%); LC/MS: m/z 524 [M+H]⁺, Rt 3.6 min.

Intermediates 65 to 72 of formula (VIIa) were prepared by methods analogous to that described for Intermediate 64 using the corresponding aminobenzophenone (see Table 15) for which previous synthesis have already been reported in the literature.

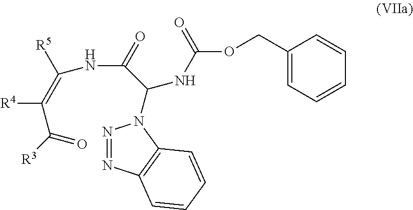

TABLE 15

| Int | R³ | R⁴/R⁵ | from Int | Physical data |
|---|---|---|---|---|
| 65 | 4-Cl—-phenyl | (R⁴)—(CH)₄—(R⁵) | (2-aminophenyl)[4- | LC/MS: m/z 538 |
| | | | (chloro)phenyl]- | [M − H]⁻; Rt 3.61 |
| | | | methanone (TCl-US) | min. |
| 66 | 4-MeO—phenyl | (R⁴)—(CH)₄—(R⁵) | (2-aminophenyl)[4- | LC/MS: m/z 534 |
| | | | (methyloxy)phenyl]- | [M − H]⁻; |
| | | | methanone (J. | Rt 3.46 min. |
| | | | Chem. Soc. (Perkins 1) | |
| | | | (1983), (9), 2077- | |
| | | | 87). | |
| 67 | 4-tolyl | (R⁴)—(CH)₄—(R⁵) | (2-aminophenyl)(4- | LC/MS: m/z 518 |
| | | | methylphenyl)- | [M − H]⁻, |
| | | | methanone (ACROS) | Rt 3.58 min. |
| 68 | 3-MeO—phenyl | (R⁴)—(CH)₄—(R⁵) | (2-aminophenyl)[3- | LC/MS: m/z 536 |
| | | | (methyloxy)phenyl]- | [M + H]⁺ Rt. 3.51 |
| | | | methanone (J. | min. |
| | | | Chem. Soc. (Perkins 1), | |
| | | | (1972), (20), 2524-6) | |
| 69 | phenyl | (R⁴)—(CH)₂—C(Me)— | (2-amino-4-methyl- | LC/MS: m/z 518 |
| | | CH—(R⁵) | phenyl)(phenyl)- | [M − H]⁻, |
| | | | methanone (Aldrich) | Rt 3.62 min. |
| 70 | phenyl | (R⁴)—CH—CH—S—(R⁵) | (2-amino-3-thienyl)- | LC/MS: m/z 512 |
| | | | (phenyl)-methanone | [M + H]⁺ , Rt |
| | | | (J. Med. Chem., | 3.66 min |
| | | | (2002), 45, 387) | |
| 71 | phenyl | (R⁴)—S—CH—CH—(R⁵) | (3-amino-2-thienyl)- | LC/MS: m/z 512 |
| | | | (phenyl)methanone | [M + H]⁺, Rt 3.56 |
| | | | (Monatsh. Chem. | min. |
| | | | (1973), 104(5), 1343-7) | |
| 72 | phenyl | R⁴)—CH—C(OMe)— | [2-amino-5- | No LC/MS |
| | | (CH)₂—(R⁵) | (methyloxy)phenyl] | |
| | | | (phenyl)methanone | |
| | | | (J.Org.Chem. (1991), | |
| | | | 56, 3752-3755) | |

### Intermediate 73: phenylmethyl (1-(1H-1,2,3-benzotriazol-1-yl)-2-{[2-[(4-chlorophenyl)carbonyl]-4-(methyloxy)phenyl]amino}-2-oxoethyl)carbamate

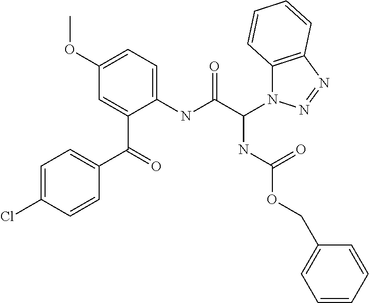

To a suspension of 1H-1,2,3-benzotriazol-1-yl({[(phenylmethyl)oxy]carbonyl}amino)acetic acid (17.7 g, 54.29 mmol) in THF (170 mL) at −10° C., was added dropwise oxalyl chloride (27 mL, 65.15 mmol, 1.2 equiv.). After 5 min, DMF was added (500 μL), the reaction mixture was stirred at 0° C. for 3 h. Then, at this temperature, a solution of Intermediate 80 (11.8 g, 54.29 mmol, 1 equiv.) and N-methyl morpholine (5 mL, 54.29 mmol, 1 equiv.) in THF (100 mL) was added dropwise. The resulting mixture was stirred at RT for 2 days. The precipitate formed was filtered, washed with water and dried to give the title compound as a yellow solid which was used in the next step without further purification.

### Intermediate 74: phenylmethyl {1-(1H-1,2,3-benzotriazol-1-yl)-2-[(2-{[4-(methyloxy)phenyl]carbonyl}phenyl)amino]-2-oxoethyl}carbamate

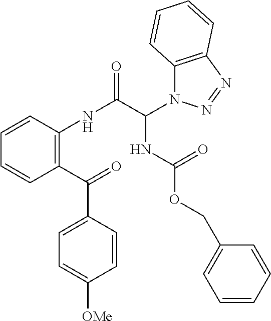

Intermediate 78 (13.2 g, 58.1 mmol) and 1H-1,2,3-benzotriazol-1-yl{[(benzyloxy)carbonyl]amino}acetic acid (28.4 g, 87.0 mmol 1.5 equiv.) in dry DCM (790 mL) were cooled to 0° C. under nitrogen. EDCI (16.7 g, 87.0 mmol, 1.5 equiv.) and 4-DMAP (0.7 g, 5.8 mmol, 0.1 equiv.) were added at 0° C. and the resulting solution was stirred for 1 h. The reaction was washed with saturated sodium bicarbonate solution (2×300 mL) and brine (2×300 mL). The organic layer was dried over Na₂SO₄ and the solvent removed in vacuo to give the title compound as a crude product which was used in the next step without purification;

LC/MS: m/z 417 [M-Bt]⁺, Rt 2.90 min.

### Intermediate 75: phenylmethyl (1-(1H-1,2,3-benzotriazol-1-yl)-2-oxo-2-{[2-(phenyl carbonyl)-1-benzofuran-3-yl]amino}ethyl)carbamate

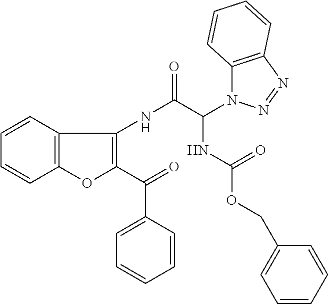

1H-1,2,3-benzotriazol-1-yl{[(benzyloxy)carbonyl]amino}acetic acid (prepared according to J. Org. Chem. 1990, 55, 2206) (6.9 g, 21.1 mmol, 1.0 equiv.) in dry THF (60 mL) was cooled to 0° C. under nitrogen and oxalyl chloride (11.6 mL, 23.2 mmol, 1.1 equiv.) was added dropwise followed by the addition of dry DMF (4 drops). The reaction mixture was stirred for 2 h at this temperature before a solution of Intermediate 81 (5.0 g, 21.1 mmol) and NMM (2.3 mL, 21.1 mmol, 1.0 equiv.) in dry THF (20 mL) was added dropwise. The resulting solution was allowed to warm to RT and stirred for 18 h before being filtered. The filtrate was concentrated under reduced pressure to give the title compound as a crude product which was used in the next step without purification.

### Intermediate 76: ethyl [1-(1H-1,2,3-benzotriazol-1-yl)-2-({2-[(4-chlorophenyl)carbonyl]-1-benzofuran-3-yl}amino)-2-oxoethyl]carbamate

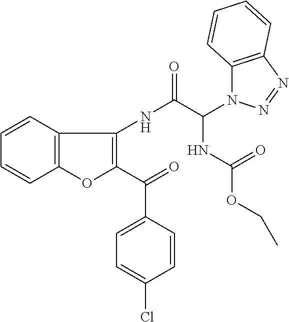

1H-1,2,3-benzotriazol-1-yl{[(ethyloxy)carbonyl]amino}acetic acid (prepared according to J. Org. Chem. 1990, 55, 2206) (25.3 g, 95.7 mmol, 1.3 equiv.) in dry THF (500 mL) was cooled to 0° C. under nitrogen and oxalyl chloride (51.5 mL, 0.1 mol, 1.4 equiv.) was added dropwise followed by the addition of dry DMF (4 drops). The reaction mixture was stirred for 2 h at this temperature before a solution of Intermediate 82 (20.0 g, 73.6 mmol) and NMM (10.5 mL, 95.7 mmol, 1.3 equiv.) in dry THF (400 mL) was added dropwise. The resulting solution was allowed to warm to RT and stirred for 18 h before being filtered. The filtrate was concentrated under reduced pressure to give the title compound as a crude product which was used in the next step without purification.

### Intermediate 77: ethyl [1-(1H-1,2,3-benzotriazol-1-yl)-2-({2-[(4-fluorophenyl)carbonyl]phenyl}amino)-2-oxoethyl]carbamate

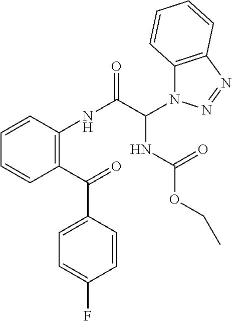

(2-Aminophenyl)(4-fluorophenyl)methanone (prepared according to WO00/05195) (10.8 g, 50.2 mmol) and 1H-1,2,3-benzotriazol-1-yl{[(ethyloxy)carbonyl]amino}acetic acid (19.9 g, 75.3 mmol 1.5 equiv.) in dry DCM (680 mL) were cooled to 0° C. under nitrogen. EDCI (14.4 g, 75.3 mmol, 1.5 equiv.) and i-Pr₂NEt (13.1 mL, 75.3 mmol, 1.5 equiv.) were added at 0° C. and the resulting solution was allowed to warm up to RT and stirred for 18 h. The reaction was washed with a saturated sodium bicarbonate solution (2×200 mL) and brine (2×200 mL). The organic layer was dried over Na₂SO₄ and the solvent removed in vacuo to give the title compound as a crude product which was used in the next step without purification.

### Intermediate 78: (2-aminophenyl)[4-(methyloxy)phenyl]methanone

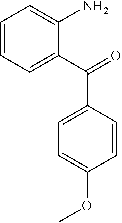

To a solution of 2-amino-N-methyl-N-(methyloxy)benzamide acid (prepared according to J. Org. Chem. 1991, 56, 3750) (25.6 g, 0.14 mol) and 4-bromoanisol (17.8 mL, 0.14 mol) in dry THF (830 mL) at −78° C. was added dropwise n-BuLi (182.0 mL, 1.6M in hexanes, 0.29 mol, 2.05 equiv.) for 2 h. When addition was done, the reaction mixture was stirred at this temperature for 0.5 h before being quenched with aqueous 1.2N HCl (256 mL). The aqueous layer was extracted with EtOAc (3×300 mL) and the combined organic layers were washed with water (500 mL), brine (500 mL), dried over Na₂SO₄, filtered and concentrated under reduced pressure. Purification by flash-chromatography (cyclohexane/ether:8/2 then 1/1) gave the title compound (22.7 g, 70%) as a dark orange oil; ¹H NMR (300 MHz, CDCl₃) δ ppm: 7.56 (m, 2H), 7.34 (m, 1H), 7.15 (m, 1H), 6.83 (m, 2H), 6.61 (m, 1H), 6.50 (m, 1H), 5.74 (br s, 2H), 3.75 (s, 3H).

### Intermediate 79: [2-amino-5-(methyloxy)phenyl](4-chlorophenyl)methanone

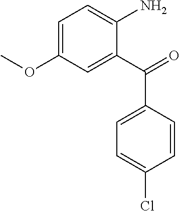

To a solution of Intermediate 83 (40.0 g, 0.21 mol, 1 equiv.) in a toluene/ether (2/1) mixture (760 mL) at 0° C. was added dropwise a solution of 4-chlorophenylmagnesium bromide (170 mL, 1M in Et₂O, 0.17 mol, 0.8 equiv.). The reaction mixture was allowed to warm to RT and stirred for 1 h before being quenched with 1N HCl (200 mL). The aqueous layer was extracted with EtOAc (3×150 mL) and the combined organics were washed with brine (100 mL), dried over Na₂SO₄, filtered and concentrated under reduced pressure. The crude compound was then dissolved in EtOH (400 mL) and 6N HCl (160 mL) was added. The reaction mixture was refluxed for 2 h before being concentrated to one-third in volume. The resulting solid was filtered and washed twice with ether before being suspended in EtOAc and neutralised with 1N NaOH. The aqueous layer was extracted with EtOAc (3×150 mL) and the combined organics were washed with brine (150 mL), dried over Na₂SO₄, filtered and concentrated under reduced pressure. The title compound was obtained as a yellow solid (39 g, 88% yield); LC/MS: m/z 262 [M+H]+, Rt 2.57 min.

### Intermediate 80: (3-amino-1-benzofuran-2-yl)(phenyl)methanone

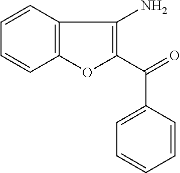

To a solution of 2-cyanophenol (24.5 g, 0.2 mol) and 2-bromoacetophenone (40.9 g, 0.2 mol) in acetone (1 L) was added potassium carbonate (85.2 g, 0.6 mol, 3 equiv.). The reaction mixture was heated to 60° C. under nitrogen for 18 h and then allowed to cool to RT. K₂CO₃ was filtered off, washed with acetone (100 ml) and filtrate was concentrated under reduced pressure to give the title compound (48.8 g, 100%) as a yellow solid; m.p. 122-124° C.; ¹H NMR (300 MHz, CDCl₃) δ ppm: 8.17 (dd, 2H, J=7.7 and 1.8 Hz), 7.56 (d, 1H, J=7.9 Hz), 7.51-7.36 (m, 5H), 7.19 (m, 1H), 5.20 (br s, 2H); LC/MS: m/z 238 [M+H]⁺, Rt 2.59 min.

### Intermediate 81: (3-amino-1-benzofuran-2-yl)(4-chlorophenyl)methanone

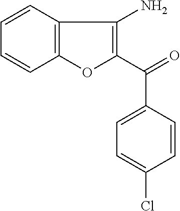

To a solution of 2-cyanophenol (15.0 g, 0.12 mol) and 2-bromo-4′-chloroacetophenone (29.4 g, 0.12 mol) in acetone (500 mL) was added potassium carbonate (52.2 g, 0.38 mol, 3 equiv.). The reaction mixture was heated to reflux under nitrogen for 18 h and then allowed to cool to RT. K₂CO₃ was filtered off, washed with acetone (100 ml) and DCM (100 mL) and filtrate was concentrated under reduced pressure. The crude solid was then washed with cold CH₃CN to give after filtration the title compound (21.6 g, 63%) as a yellow solid; R_{f}=0.80 (DCM/MeOH:98/2); m.p. 202-204° C.; ¹H NMR (300 MHz, CDCl₃) δ ppm: 8.20 (d, 2H, J=8.4 Hz), 7.63 (d, 1H, J=7.9 Hz), 7.62-7.41 (m, 4H), 7.28 (m, 1H), 6.05 (br s, 2H); LC/MS: m/z 272 [M+H]⁺, Rt 2.97 min.

### Intermediate 82: (2-amino-5-iodophenyl)(phenyl)methanone

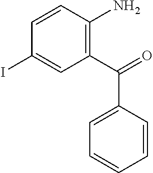

To a solution of 2-aminobenzophenone (50 g, 263 mmol) in anhydrous DCM (800 mL) at −70° C. was added iodine monochloride (49.4 g, 305 mmol, 1.2 equiv.). The reaction mixture was stirred at −60° C. for 5 h, then overnight at RT. The resulting mixture was hydrolyzed with saturated aqueous Na₂SO₄ and then extracted with DCM. The resulting solid was filtered to give the title compound which was used without further purification.

### Intermediate 83: 2-methyl-6-(methyloxy)-4H-3,1-benzoxazin-4-one

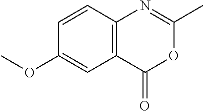

A solution of 5-methoxyanthranilic acid (Lancaster) (41.8 g, 0.25 mol) was heated under reflux in acetic anhydride (230 mL) for 3.5 h before being concentrated under reduced pressure. The crude compound was then concentrated twice in the presence of toluene and then filtered, The resulting solid was washed twice with ether to yield to the title compound (33.7 g, 71% yield) as a brown solid; LC/MS: m/z 192 [M+H]⁺, Rt 1.69 min.

### Intermediate 84: 1-(4-Fluorophenyl)ethyl 4-nitrophenyl carbonate

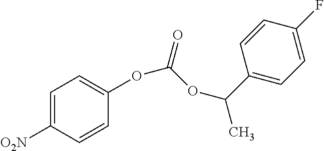

A solution of 4-nitrophenylchloroformate (673 mg, 3.34 mmol) in dry DCM (10 mL) was added dropwise to a solution of 1-(4-fluorophenyl)ethanol (468 mg, 3.34 mmol) and pyridine (283 μl, 3.5 mmol) in dry DCM (10 mL) at 0° C. under nitrogen and the mixture was stirred at 0° C. for one hour and then at RT for 2 days. The mixture was washed with 2N HCl and the layers separated. The organic extract was diluted with cyclohexane and applied to a 10 g Si SPE cartridge. Elution with DCM/cyclohexane (1:1) followed by DCM gave the title compound (180 mg, 18%), LC/MS: m/z 306 [M+H]⁺, Rt 3.5 min.

Intermediates 85 to 87 of formula (X) were prepared by methods analogous to that described for Intermediate 84 using the starting materials indicated (see Table 16).

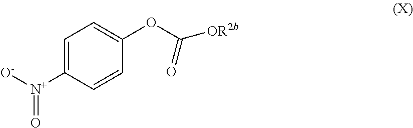

TABLE 16

| Int | R¹ | from Int. | Physical data |
|---|---|---|---|
| 85 | 4-fluorobenzyl | 4- | Purification by Biotage TM |
| | | fluoro- | chromatography (Silica, 40 g) |
| | | benzyl- | eluting with 17:3 |
| | | alcohol | cyclohexane/EtOAc |
| | | | colourless gum; LC/MS: |
| | | | m/z (no ion) [M + H]⁺; |
| | | | Rt 3.39 min. |
| 86 | 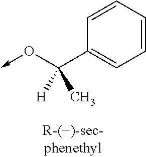 | R-(+)-sec- phenethyl alcohol | Purification by Biotage TM chromatography (Silica, 40 g) eluting with 17:3 cyclohexane/ EtOAc; pale yellow gum; LC/MS: m/z (no ion) [M + H]⁺; Rt 3.46 min. |
| 87 | 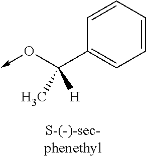 | S-(−)-sec- phenethyl alcohol | Purification by Biotage TM chromatography (Silica, 40 g) eluting with 17:3 cyclohexane/EtOAc; pale yellow gum; LC/MS: m/z (no ion) [M + H]⁺, Rt 3.46 min. |

### Intermediate 88: 4-nitrophenyl [3-(phenylcarbonyl)phenyl]carbamate

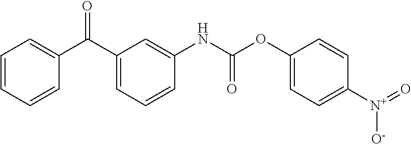

To a solution of 4-aminobenzophenone (3 g, 15.2 mmol) in DCM (250 mL) was added DMAP (1 g). A solution of 4-nitrophenyl chloridocarbonate (9.2 g, 3 equiv.) in DCM was added then dropwise and the reaction mixture was stirred at RT for 1 h. The precipitate was filtered, washed with DCM and diisopropyl ether to give a cream solid (1.8 g) which was used without further purification.

Experimental details of LC/MS methods D and F as referred to herein are as follows:

LC/MS (Method D) was conducted on a Supelcosil LCABZ+PLUS column (3 μm, 3.3 cm×4.6 mm ID) eluting with 0.1% HCO₂H and 0.01 M ammonium acetate in water (solvent A), and 95% acetonitrile and 0.05% HCO₂H in water (solvent B), using the following elution gradient 0-0.7 minutes 0% B, 0.7-4.2 minutes 0→100% B, 4.2-5.3 minutes 100% B, 5.3-5.5 minutes 100→0% B at a flow rate of 3 mL/minute. The mass spectra (MS) were recorded on a Fisons VG Platform mass spectrometer using electrospray positive ionisation [(ES+ve to give [M+H]⁺ and [M+NH₄]⁺ molecular ions] or electrospray negative ionisation [(ES−ve to give [M−H]- molecular ion] modes. Analytical data from this apparatus are given with the following format: [M+H]⁺ or [M−H]⁻.

LC/MS (Method F) was conducted on an Sunfire C18 column (30 mm×4.6 mm i.d. 3.5 μm packing diameter) at 30 degrees centigrade, eluting with 0.1% v/v solution of Trifluoroacetic Acid in Water (Solvent A) and 0.1% v/v solution of Trifluoroacetic Acid in Acetonitrile (Solvent B) using the following elution gradient 0-0.1 min 3% B, 0.1-4.2 min 3-100% B, 4.2-4.8 min 100% B, 4.8-4.9 min 100-3% B, 4.9-5.0 min 3% B at a flow rate of 3 ml/min. The UV detection was an averaged signal from wavelength of 210 nm to 350 nm and mass spectra were recorded on a mass spectrometer using positive electrospray ionization. Ionisation data was rounded to the nearest integer.

LC/HRMS: Analytical HPLC was conducted on a Uptisphere-hsc column (3 μm 33×3 mm id) eluting with 0.01M ammonium acetate in water (solvent A) and 100% acetonitrile (solvent B), using the following elution gradient 0-0.5 minutes 5% B, 0.5-3.75 minutes 5→100% B, 3.75-4.5 100% B, 4.5-5 100→5% B, 5-5.5 5% B at a flow rate of 1.3 mL/minute. The mass spectra (MS) were recorded on a micromass LCT mass spectrometer using electrospray positive ionisation [ES+ve to give MH⁺ molecular ions] or electrospray negative ionisation [ES−ve to give (M−H)⁻ molecular ions] modes.

TLC (thin layer chromatography) refers to the use of TLC plates sold by Merck coated with silica gel 60 F254.

Silica chromatography techniques include either automated (Flashmaster or Biotage SP4) techniques or manual chromatography on pre-packed cartridges (SPE) or manually-packed flash columns.

When the name of a commercial supplier is given after the name of a compound or a reagent, for instance “compound X (Aldrich)” or “compound X/Aldrich”, this means that compound X is obtainable from a commercial supplier, such as the commercial supplier named.

### Reference compound A: 2-methyl-6-(methyloxy)-4H-3,1-benzoxazin-4-one

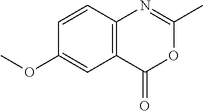

A solution of 5-methoxyanthranilic acid (Lancaster) (41.8 g, 0.25 mol) was refluxed in acetic anhydride (230 mL) for 3.5 h before being concentrated under reduced pressure. The crude compound was then concentrated twice in the presence of toluene before being filtered and washed twice with ether to yield to the title compound (33.7 g, 71% yield) as a brown solid; LC/MS (Method D): m/z 192 [M+H]⁺, Rt 1.69 min.

### Reference compound B: [2-amino-5-(methyloxy)phenyl](4-chlorophenyl)methanone

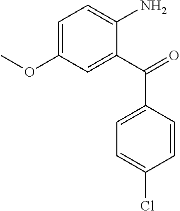

To a solution of 2-methyl-6-(methyloxy)-4H-3,1-benzoxazin-4-one (for a preparation see Reference compound A) (40.0 g, 0.21 mol) in a toluene/ether (2/1) mixture (760 mL) at 0° C. was added dropwise a solution of 4-chlorophenylmagnesium bromide (170 mL, 1M in Et₂O, 0.17 mol). The reaction mixture was allowed to warm to room temperature and stirred for 1 h before being quenched with 1N HCl (200 mL). The aqueous layer was extracted with EtOAc (3×150 mL) and the combined organics were washed with brine (100 mL), dried over Na₂SO₄, filtered and concentrated under reduced pressure. The crude compound was then dissolved in EtOH (400 mL) and 6N HCl (160 mL) was added. The reaction mixture was refluxed for 2 h before being concentrated to one-third in volume. The resulting solid was filtered and washed twice with ether before being suspended in EtOAc and neutralised with 1N NaOH. The aqueous layer was extracted with EtOAc (3×150 mL) and the combined organics were washed with brine (150 mL), dried over Na₂SO₄, filtered and concentrated under reduced pressure. The title compound was obtained as a yellow solid (39 g, 88% yield); LC/MS (Method D): m/z 262 [M+H]+, Rt 2.57 min.

### Reference Compound C: Methyl N¹-[2-[(4-chlorophenyl)carbonyl]-4-(methyloxy)phenyl]-N²-{[(9H-fluoren-9-ylmethyl)oxy]carbonyl}-L-α-asparaginate

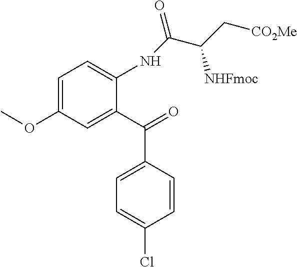

Methyl N-{[(9H-fluoren-9-ylmethyl)oxy]carbonyl}-L-α-aspartyl chloride (Int. J. Peptide Protein Res. 1992, 40, 13-18) (93 g, 0.24 mol) was dissolved in CHCl₃ (270 mL) and [2-amino-5-(methyloxy)phenyl](4-chlorophenyl)methanone (for a preparation see Reference compound B) (53 g, 0.2 mol) was added. The resulting mixture was stirred at 60° C. for 1 h before being cooled and concentrated at 60% in volume. Ether was added at 0° C. and the resulting precipitate was filtered and discarded. The filtrate was concentrated under reduced pressure and used without further purification.

### Reference compound D: Methyl [(3S)-5-(4-chlorophenyl)-7-(methyloxy)-2-oxo-2,3-dihydro-1H-1,4-benzodiazepin-3-yl]acetate

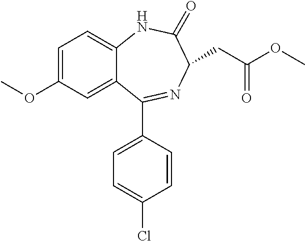

To a solution of Methyl N1-[2-[(4-chlorophenyl)carbonyl]-4-(methyloxy)phenyl]-N2-{[(9H-fluoren-9-ylmethyl)oxy]carbonyl}-L-α-asparaginate (for a preparation see Reference compound C) (assumed 0.2 mol) in DCM (500 mL) was added Et₃N (500 mL, 3.65 mol) and the resulting mixture was refluxed for 24 h before being concentrated. The resulting crude amine was dissolved in 1,2-DCE (1.5 L) and AcOH (104 mL, 1.8 mol) was added carefully. The reaction mixture was then stirred at 60° C. for 2 h before being concentrated in vacuo and dissolved in DCM. The organic layer was washed with 1N HCl and the aqueous layer was extracted with DCM (×3). The combined organic layers were washed twice with water, and brine, dried over Na₂SO₄, filtered and concentrated under reduced pressure. The crude solid was recrystallised in MeCN leading to the title compound (51 g) as a pale yellow solid. The filtrate could be concentrated and recrystallised in MeCN to give to another 10 g of the desired product R_{f}=0.34 (DCM/MeOH:95/5). HRMS (M+H)⁺ calculated for C₁₉H₁₈³⁵ClN₂O₄ 373.0955; found 373.0957.

### Reference compound E: Methyl [(3S)-5-(4-chlorophenyl)-7-(methyloxy)-2-thioxo-2,3-dihydro-1H-1,4-benzodiazepin-3-yl]acetate

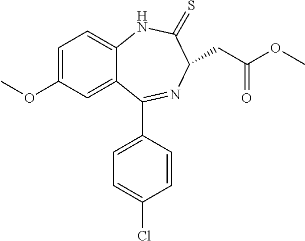

A suspension of P₄S₁₀ (36.1 g, 81.1 mmol) and Na₂CO₃ (8.6 g, 81.1 mmol) in 1,2-DCE (700 mL) at room temperature was stirred for 2 h before Methyl [(3S)-5-(4-chlorophenyl)-7-(methyloxy)-2-oxo-2,3-dihydro-1H-1,4-benzodiazepin-3-yl]acetate (for a preparation see Reference compound D) (16.8 g, 45.1 mmol) was added. The resulting mixture was stirred at 70° C. for 2 h before being cooled and filtered. The solid was washed twice with DCM and the filtrate washed with sat. NaHCO₃ and brine. The organic layer was dried over Na₂SO₄, filtered and concentrated under reduced pressure. The crude product was purified by flash-chromatography on silica gel (DCM/MeO:99/1) to afford the title compound (17.2 g, 98% yield) as a yellowish solid. LC/MS (Method D): m/z 389 [M(³⁵Cl)+H]⁺, Rt 2.64 min

HRMS (M+H)⁺ calculated for C₁₉H₁₈³⁵ClN₂O₃S 389.0727; found 389.0714.

### Reference compound F: Methyl [(3S)-2-[(1Z)-2-acetylhydrazino]-5-(4-chlorophenyl)-7-(methyloxy)-3H-1,4-benzodiazepin-3-yl]acetate

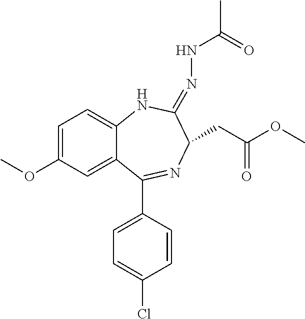

To a suspension of Methyl [(3S)-5-(4-chlorophenyl)-7-(methyloxy)-2-thioxo-2,3-dihydro-1H-1,4-benzodiazepin-3-yl]acetate (for a preparation see Reference compound E (9.0 g, 23.2 mmol) in THF (300 mL) at 0° C. was added hydrazine monohydrate (3.4 mL, 69.6 mmol) dropwise. The reaction mixture was stirred for 5 h between 5° C. and 15° C. before being cooled at 0° C. Et₃N (9.7 mL, 69.6 mmol) was then added slowly and acetyl chloride (7.95 mL, 69.6 mmol) was added dropwise. The mixture was then allowed to warm to room temperature for 16 h before being concentrated under reduced pressure. The crude product was dissolved in DCM and washed with water. The organic layer was dried over Na₂SO₄, filtered and concentrated in vacuo to give the crude title compound (9.7 g, 98% yield) which was used without further purification. R_{f}=0.49 (DCM/MeOH:90/10).

### Reference compound G: Methyl [(4S)-6-(4-chlorophenyl)-1-methyl-8-(methyloxy)-4H-[1,2,4]triazolo[4,3-a][1,4]benzodiazepin-4-yl]acetate

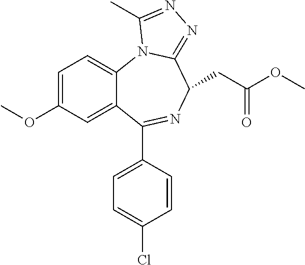

The crude Methyl [(3S)-2-[(1Z)-2-acetylhydrazino]-5-(4-chlorophenyl)-7-(methyloxy)-3H-1,4-benzodiazepin-3-yl]acetate (for a preparation see Reference compound F) (assumed 9.7 g) was suspended in THF (100 ml) and AcOH (60 mL) was added at room temperature. The reaction mixture was stirred at this temperature for 2 days before being concentrated under reduced pressure. The crude solid was triturated in i-Pr₂O and filtered to give the title compound (8.7 g, 91% over 3 steps) as an off-white solid.

HRMS (M+H)⁺ calculated for C₂₁H₂₀ClN₄O₃ 411.1229; found 411.1245.

### Reference compound H: [(4S)-6-(4-Chlorophenyl)-1-methyl-8-(methyloxy)-4H-[1,2,4]triazolo[4,3-a][1,4]benzodiazepin-4-yl]acetic acid

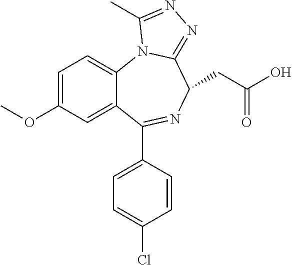

To a solution of Methyl [(4S)-6-(4-chlorophenyl)-1-methyl-8-(methyloxy)-4H-[1,2,4]triazolo[4,3-a][1,4]benzodiazepin-4-yl]acetate (for a preparation see Reference compound G) (7.4 g, 18.1 mmol) in THF (130 mL) at room temperature was added 1N NaOH (36.2 mL, 36.2 mmol). The reaction mixture was stirred at this temperature for 5 h before being quenched with 1N HCl (36.2 mL) and concentrated in vacuo. Water is then added and the aqueous layer was extracted with DCM (×3) and the combined organic layers were dried over Na₂SO₄, filtered and concentrated under reduced pressure to give the title compound (7 g, 98% yield) as a pale yellow solid.

### Reference compound H: 1,1-dimethylethyl [5-({[(4S)-6-(4-chlorophenyl)-1-methyl-8-(methyloxy)-4H-[1,2,4]triazolo[4,3-a][1,4] benzodiazepin-4-yl]acetyl}amino)pentyl]carbamate

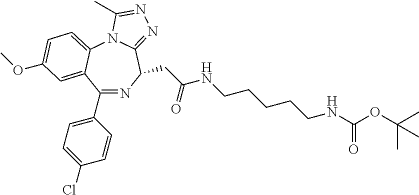

A mixture of [(4S)-6-(4-chlorophenyl)-1-methyl-8-(methyloxy)-4H-[1,2,4]triazolo[4,3-a][1,4]benzodiazepin-4-yl]acetic acid (for a preparation see Reference compound G) (1.0 g, 2.5 mmol), HATU (1.9 g, 5 mmol) and DIPEA (0.88 ml, 5 mmol) was stirred for 80 minutes at room temperature, to this was added 1,1-dimethylethyl (4-aminobutyl)carbamate (1.05 ml, 5.0 mmol, available from Aldrich). The reaction mixture was stirred at room temperature for 2 h before it was concentrated. The residue was taken up in dichloromethane and washed with 1N HCl. The aqueous layer was extracted with dichloromethane twice. Organic layer was washed with 1N sodium hydroxide, followed by a saturated solution of sodium chloride, dried over sodium sulphate and concentrated. The residue was purified by flash-chromatography on silica using dichloromethane/methanol 95/5 to give the title compound as a yellow solid (1.2 g). LC/MS (Method D): rt=3.04 min.

### Reference compound J: N-(5-aminopentyl)-2-[(4S)-6-(4-chlorophenyl)-1-methyl-8-(methyloxy)-4H-[1,2,4]triazolo[4,3-a][1,4]benzodiazepin-4-yl]acetamide trifluoroacetate

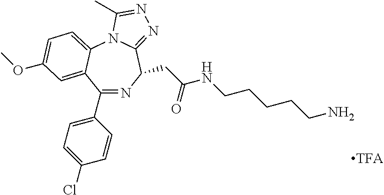

To a solution of 1,1-dimethylethyl [5-({[(4S)-6-(4-chlorophenyl)-1-methyl-8-(methyloxy)-4H-[1,2,4]triazolo[4,3-a][1,4]benzodiazepin-4-yl]acetyl}amino)pentyl]carbamate (for a preparation see Reference compound H) (0.2 g, 0.34 mmol) in dichloromethane (3 ml) was added trifluoroacetic acid (0.053 ml, 0.68 mmol) dropwise at 0° C. The reaction mixture was stirred for 3 h from 0° C. to room temperature. The reaction mixture was concentrated to dryness to afford the title compound as a hygroscopic yellow oil (200 mg) LC/MS (Method D): rt=2.33 min.

HRMS (M+H)⁺ calculated for C₂₅H₂₉ClN₆O₂ 481.2119; found 481.2162.

### Reference compound K: Mixture of 5- and 6-isomers of Alexa Fluor 488-N-(5-aminopentyl)-2-[(4S)-6-(4-chlorophenyl)-1-methyl-8-(methyloxy)-4H-[1,2,4]triazolo[4,3-a][1,4]benzodiazepin-4-yl]acetamide

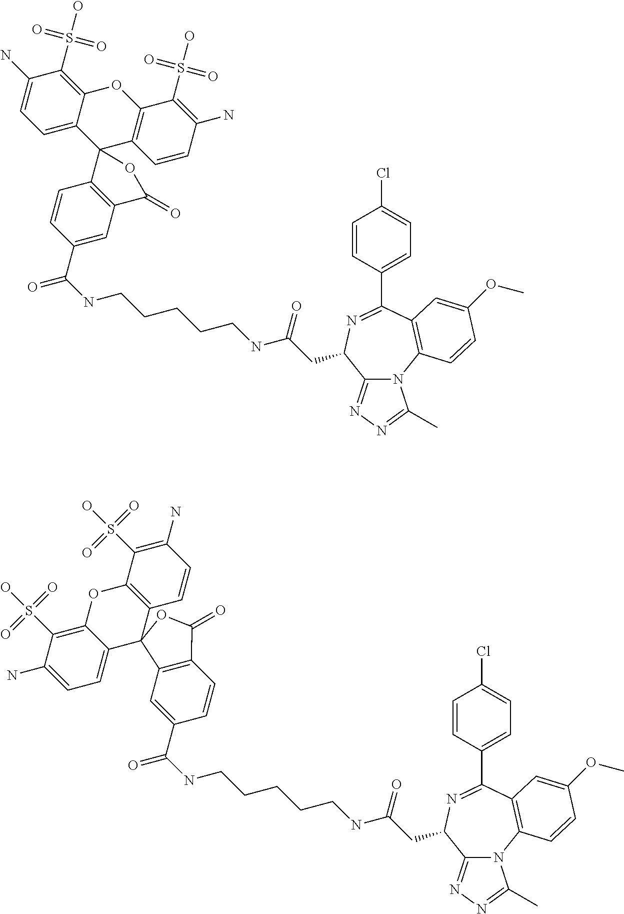

N-(5-aminopentyl)-2-[(4S)-6-(4-chlorophenyl)-1-methyl-8-(methyloxy)-4H-[1,2,4]triazolo[4,3-a][1,4]benzodiazepin-4-yl]acetamide trifluoroacetate (for a preparation see Reference compound J)(7.65 mg, 0.013 mmol) was dissolved in N,N-Dimethylformamide (DMF) (300 μl) and added to Alexa Fluor 488 carboxylic acid succinimidyl ester (5 mg, 7.77 μmol, mixture of 5 and 6 isomers, available from Invitrogen, product number A-20100) in an Eppendorf centrifuge tube. Hunig's base (7.0 μl, 0.040 mmol) was added and the mixture vortex mixed overnight. After 18 h the reaction mixture was evaporated to dryness and the residue redissolved in DMSO/water (50%, <1 ml total), applied to a preparative Phenomenex Jupiter C18 column and eluted with a gradient of 95% A: 5% B to 100% B (A=0.1% trifluoroacetic acid in water, B=0.1% TFA/90% acetonitrile/10% water) at a flow rate of 10 ml/min over 150 minutes. Impure fractions were combined and re-purified using the same system. Fractions were combined and evaporated to yield the title product (2.8 mg) as a mixture of the 2 regioisomers shown. LC/MS (Method F): MH+=999, rt=1.88 min.

### Biological Test Methods

### Fluorescence Anisotropy Binding Assay

The binding of the compounds of formula (I) to Bromodomain 2, 3 and 4 was assessed using a Fluorescence Anisotropy Binding Assay.

The Bromodomain protein, fluorescent ligand (Reference compound K see above) and a variable concentration of test compound are incubated together to reach thermodynamic equilibrium under conditions such that in the absence of test compound the fluorescent ligand is significantly (>50%) bound and in the presence of a sufficient concentration of a potent inhibitor the anisotropy of the unbound fluorescent ligand is measurably different from the bound value.

All data was normalized to the mean of 16 high and 16 low control wells on each plate. A four parameter curve fit of the following form was then applied:

y=a+((b−a)/(1+(10̂x/10̂c)̂d)

Where ‘a’ is the minimum, ‘b’ is the Hill slope, ‘c’ is the pIC50 and ‘c’ is the maximum.

Recombinant Human Bromodomains (Bromodomain 2 (1-473), Bromodomain 3 (1-435) and Bromodomain 4 (1-477)) were expressed in E. coli cells (in pET15b vector) with a six-His tag at the N-terminal. The His-tagged Bromodomain was extracted from E. coli cells using 0.1 mg/ml lysozyme and sonication. The Bromodomain was then purified by affinity chromatography on a HisTRAP HP column, eluting with a linear 10-500 mM Imidazole gradient, over 20 Cv. Further purification was completed by Superdex 200 prep grade size exclusion column. Purified protein was stored at −80 C in 20 mM HEPES pH 7.5 and 100 mM NaCl.

Protocol for Bromodomain 2: All components were dissolved in buffer composition of 50 mM HEPES pH7.4, 150 mm NaCl and 0.5 mM CHAPS with final concentrations of Bromodomain 2, 75 nM, fluorescent ligand 5 nM. 10 μl of this reaction mixture was added using a micro multidrop to wells containing 100 nl of various concentrations of test compound or DMSO vehicle (1% final) in Greiner 384 well Black low volume microtitre plate and equilibrated in the dark for 60 mins at room temperature. Fluorescence anisotropy was read in Envision (λex=485 nm, λEM=530 nm; Dichroic −505 nM).

Protocol for Bromodoamin 3: All components were dissolved in buffer of composition 50 mM HEPES pH7.4, 150 mm NaCl and 0.5 mM CHAPS with final concentrations of Bromodomain 3, 75 nM, fluorescent ligand 5 nM. 10 μl of this reaction mixture was added using a micro multidrop to wells containing 100 nl of various concentrations of test compound or DMSO vehicle (1% final) in Greiner 384 well Black low volume microtitre plate and equilibrated in the dark for 60 mins at room temperature. Fluorescence anisotropy was read in Envision (λex=485 nm, λEM=530 nm; Dichroic −505 nM).

Protocol for Bromodomain 4: All components were dissolved in buffer of composition 50 mM HEPES pH7.4, 150 mm NaCl and 0.5 mM CHAPS with final concentrations of Bromodomain 4, 75 nM, fluorescent ligand 5 nM. 10 μl of this reaction mixture was added using a micro multidrop to wells containing 100 nl of various concentrations of test compound or DMSO vehicle (1% final) in Greiner 384 well Black low volume microtitre plate and equilibrated in the dark for 60 mins at room temperature. Fluorescence anisotropy was read in Envision (λex=485 nm, λEM=530 nm; Dichroic −505 nM).

Examples 3-12, 15, 17, 18, 20, 23, 24, 25, 27-34, 36, 44, 53, 54, 56-59, 61, 64, 65, 70, 71, 75, 76, 79, 81 and 85-125 were tested in the assays described above and were found to have a pIC50 ≦5.0 in one or more of the BRD2, BRD3 and BRD4 assays with the exception of example 44, and Examples 117-125 which had a pIC50≧5.0. Examples 4, 6-9, 12, 24, 27, 28, 29, 31, 32, 34, 36, 53, 54, 56, 57, 58, 61, 64, 65, 70, 71, 75, 85-89 and 97 had a pIC50≧6.0 in one or more of the BRD2, BRD3 and BRD4 assays described above.

### LPS Stimulated Whole Blood Measuring TNFα Levels Assay

Activation of monocytic cells by agonists of toll-like receptors such as bacterial lipopolysaccharide (LPS) results in production of key inflammatory mediators including TNFα. Such pathways are widely considered to be central to the pathophysiology of a range of auto-immune and inflammatory disorders.

Compounds to be tested are diluted to give a range of appropriate concentrations and 1 ul of the dilution stocks is added to wells of a 96 plate. Following addition of whole blood (130 μl) the plates are incubated at 37 degrees (5% CO2) for 30 min before the addition of 10 ul of 2.8 ug/ml LPS, diluted in complete RPMI 1640 (final concentration=200 ng/ml), to give a total volume of 140 μl per well. After further incubation for 24 hours at 37 degrees, 140 μl of PBS are added to each well. The plates are sealed, shaken for 10 minutes and then centrifuged (2500 rpm×10 min). 100 μl of the supernatant are removed and TNFα levels assayed by immunoassay (typically by MesoScale Discovery technology) either immediately or following storage at −20 degrees. Dose response curves for each compound was generated from the data and an IC50 value was calculated.

Examples 27, 28, 32, 53, 64 and 65 were tested in the above assay and were found to have a pIC50≧5.0.

These data demonstrate that the bromodomain inhibitors tested in the above assay inhibited the production of the key inflammatory mediator TNFα. This suggests that such compounds have an anti-inflammatory profile.

All publications, including but not limited to patents and patent applications, cited in this specification are herein incorporated by reference as if each individual publication were specifically and individually indicated to be incorporated by reference herein as though fully set forth.

What is claimed is:

1. A compound of formula (I) or a salt thereof 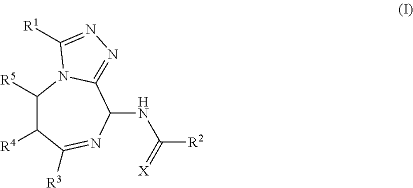
where
X is O or S;
R¹ is C₁₋₆alkyl, haloC₁₋₆alkyl, —(CH₂)ₙOR^{1a} or —(CH₂)ₘNR^{1b}R^{1c}; wherein R^{1a} is hydrogen, C₁₋₆alkyl or haloC₁₋₆alkyl; R^{1b} and R^{1c}, which may be the same or different, are hydrogen, C₁₋₆alkyl or haloC₁₋₆alkyl; and m and n, which may be the same or different, are 1, 2 or 3;
R² is R^{2a}, —OR^{2b} or —NR^{2c}R^{2d}; wherein R^{2a} and R^{2b} are carbocyclyl, carbocyclylC₁₋₄alkyl, heterocyclyl or heterocyclylC₁₋₄alkyl, or R^{2a} is carbocyclylethenyl or heterocyclylethenyl, wherein any of the carbocyclyl or heterocyclyl groups defined for R^{2a} or R^{2b} are optionally substituted by one or more groups independently selected from halogen, C₁₋₆alkyl, haloC₁₋₆alkyl, C₁₋₆alkoxy, haloC₁₋₆alkoxy, nitro, cyano, dimethylamino, benzoyl and azido; or two adjacent groups on any of the carbocyclyl or heterocyclyl groups defined for R^{2a} or R^{2b} together with the interconnecting atoms form a 5 or 6-membered ring which ring may contain 1 or 2 heteroatoms independently selected from O, S and N; or R^{2a} and R^{2b} are C₁₋₆alkyl or haloC₁₋₆alkyl; and R^{2c} and R^{2d}, which may be the same or different, are carbocyclyl, carbocyclylC₁₋₄alkyl, heterocyclyl or heterocyclylC₁₋₄alkyl, wherein any of the carbocyclyl or heterocyclyl groups defined for R^{2c} and R^{2d} are optionally substituted by one or more groups independently selected from: halogen, C₁₋₆alkyl, haloC₁₋₆alkyl, C₁₋₆alkoxy, haloC₁₋₆alkoxy, nitro, cyano and —CO₂C₁₋₄alkyl; or two adjacent groups on any of the carbocyclyl or heterocyclyl groups defined for R^{2c} and R^{2d} together with the interconnecting atoms form a 5 or 6-membered ring which ring may contain 1 or 2 heteroatoms independently selected from: O, S and N; or R^{2c} and R^{2d} are hydrogen, C₁₋₆alkyl or haloC₁₋₆alkyl;
R³ is carbocyclyl or heterocyclyl, either of which is optionally substituted independently by one or more halogen, C₁₋₆alkyl, haloC₁₋₆alkyl, C₁₋6alkoxy, haloC₁₋₆alkoxy, nitro or cyano; or R³ is C₁₋₆alkyl; and
R⁴ and R⁵ together with the interconnection carbon atoms form a benzene or aromatic heterocyclic ring, each of which is optionally substituted.

2. A compound or a salt thereof according to claim 1 in which R¹ is methyl.

3. A compound or a salt thereof according to claim 1 in which R² is —OR^{2b}.

4. A compound or a salt thereof according to claim 3 in which R^{2b} is C₁₋₆alkyl, benzyl or phenylC₁₋₆alkyl wherein benzyl is optionally substituted by fluoro.

5. A compound or a salt thereof according to claim 4 in which R^{2b} is ethyl, isopropyl, benzyl, 4-fluorobenzyl or —CH(CH₃)phenyl.

6. A compound or a salt thereof according to claim 1 in which R^{2a} is carbocycylethenyl optionally substituted by one or more groups independently selected from halogen, C₁₋₆alkyl, haloC₁₋₆alkyl, C₁₋₆alkoxy, haloC₁₋₆alkoxy, nitro, cyano, dimethylamino, benzoyl and azido.

7. A compound or a salt thereof according to claim 6 in which R^{2a} is carbocycylethenyl optionally substituted by one group selected from fluoro, chloro and methoxy.

8. A compound or a salt thereof according to claim 1 in which R^{2a} is carbocycyl or heterocyclyl optionally substituted by one or more groups independently selected from C₁₋₆alkyl, C₁₋₆alkoxy and benzoyl.

9. A compound or a salt thereof according to claim 8 in which R^{2a} is phenyl, napthylenyl or indolyl optionally substituted by one group selected from methyl, methoxy and benzoyl.

10. A compound or a salt thereof according to claim 1 in which R² is —NR^{2c}R^{2d}.

11. A compound or a salt thereof according to claim 10 in which R^{2c} is hydrogen and R^{2d} is phenyl or benzyl optionally substituted by one group selected from halogen, C₁₋₆alkyl, C₁₋₆alkoxy and —CO₂C₁₋₄alkyl.

12. A compound or a salt thereof according to claim 11 in which R^{2d} is substituted by one group selected from bromine, ethyl, methoxy and —CO₂CH₂CH₃.

13. A compound or a salt thereof according to claim 1 in which R³ is phenyl optionally substituted by one or more groups independently selected from halogen, C₁₋₆alkyl, haloC₁₋₆alkyl, C₁₋₆alkoxy, haloC₁₋₆alkoxy, nitro and cyano.

14. A compound or a salt thereof according to claim 13 in which R³ is unsubstituted phenyl.

15. A compound or a salt thereof according to claim 13 in which R³ is phenyl substituted by one group selected from methyl, chloro and methoxy.

16. A compound or a salt thereof according to claim 1 in which R⁴ and R⁵, together with the interconnecting atoms, form a benzene, a thiophene or a furan ring, any of which are optionally substituted by one or more groups independently selected from halogen, C₁₋₆alkyl, C₂₋₆alkenyl, haloC₁₋₆alkyl, C₁₋₆alkoxy, haloC₁₋₆alkoxy, nitro, cyano and heterocyclyl.

17. A compound or a salt thereof according to claim 16 in which R⁴ and R⁵, together with the interconnecting atoms, form a benzene ring, which is optionally substituted by halogen.

18. A compound or a salt thereof according to claim 1 which is the S-enantiomer.

19. A compound or a salt thereof according to claim 1 which is a compound of any one of Examples 1-84 or a salt thereof.

20. A compound or a salt thereof according to claim 1 which is a compound of any one of Examples 85-125 or a salt thereof.

21. A compound or a salt thereof according to claim 1, wherein said compound or salt is selected from the group consisting of
ethyl [6-(4-chlorophenyl)-1-methyl-4H-[1,2,4]triazolo[4,3-a][1,4]benzodiazepin-4-yl]carbamate;
phenylmethyl [1-methyl-8-(methyloxy)-6-phenyl-4H-[1,2,4]triazolo[4,3-a][1,4]benzodiazepin-4-yl]carbamate;
phenylmethyl {1-methyl-6-[4-(methyloxy)phenyl]-4H-[1,2,4]triazolo[4,3-a][1,4]benzodiazepin-4-yl}carbamate;
phenylmethyl [1-methyl-6-(4-methylphenyl)-4H-[1,2,4]triazolo[4,3-a][1,4]benzodiazepin-4-yl]carbamate;
phenylmethyl {1-methyl-6-[3-(methyloxy)phenyl]-4H-[1,2,4]triazolo[4,3-a][1,4]benzodiazepin-4-yl}carbamate;
phenylmethyl (9-methyl-4-phenyl-6H-thieno[3,2-f][1,2,4]triazolo[4,3-a][1,4]diazepin-6-yl)carbamate;
phenylmethyl (8-iodo-1-methyl-6-phenyl-4H-[1,2,4]triazolo[4,3-a][1,4]benzodiazepin-4-yl)carbamate;
(+)-phenylmethyl (1-methyl-6-phenyl-4H-[1,2,4]triazolo[4,3-a][1,4]benzodiazepin-4-yl)carbamate;
(+)-ethyl [6-(4-chlorophenyl)-1-methyl-4H-[1,2,4]triazolo[4,3-a][1,4]benzodiazepin-4-yl]carbamate;
ethyl (1-methyl-6-phenyl-4H-[1,2,4]triazolo[4,3-a][1,4]benzodiazepin-4-yl)carbamate;
ethyl {1-methyl-6-[4-(methyloxy)phenyl]-4H-[1,2,4]triazolo[4,3-a][1,4]benzodiazepin-4-yl}carbamate;
(+)-ethyl 1-methyl-6-phenyl-4H-[1,2,4]triazolo[4,3-a][1,4]benzodiazepin-4-ylcarbamate;
(4-fluorophenyl)methyl (1-methyl-6-phenyl-4H-[1,2,4]triazolo[4,3-a][1,4]benzodiazepin-4-yl)carbamate;
(1S)-1-phenylethyl (1-methyl-6-phenyl-4H-[1,2,4]triazolo[4,3-a][1,4]benzodiazepin-4-yl)carbamate;
6-(methyloxy)-N-(1-methyl-6-phenyl-4H-[1,2,4]triazolo[4,3-a][1,4]benzodiazepin-4-yl)-1H-indole-2-carboxamide;
N-(1-methyl-6-phenyl-4H-[1,2,4]triazolo[4,3-a][1,4]benzodiazepin-4-yl)-4-(phenylcarbonyl)benzamide;
(2E)-3-[4-(methyloxy)phenyl]-N-(1-methyl-6-phenyl-4H-[1,2,4]triazolo[4,3-a][1,4]benzodiazepin-4-yl)-2-propenamide;
(2E)-3-(4-chlorophenyl)-N-(1-methyl-6-phenyl-4H-[1,2,4]triazolo[4,3-a][1,4]benzodiazepin-4-yl)-2-propenamide;
(2E)-N-(1-methyl-6-phenyl-4H-[1,2,4]triazolo[4,3-a][1,4]benzodiazepin-4-yl)-3-(2-thienyl)-2-propenamide;
5-methyl-N-(1-methyl-6-phenyl-4H-[1,2,4]triazolo[4,3-a][1,4]benzodiazepin-4-yl)-1H-indole-2-carboxamide;
(2E)-N-(1-methyl-6-phenyl-4H-[1,2,4]triazolo[4,3-a][1,4]benzodiazepin-4-yl)-3-phenyl-2-propenamide;
(2E)-3-(4-fluorophenyl)-N-(1-methyl-6-phenyl-4H-[1,2,4]triazolo[4,3-a][1,4]benzodiazepin-4-yl)-2-propenamide;
N-(1-methyl-6-phenyl-4H-[1,2,4]triazolo[4,3-a][1,4]benzodiazepin-4-yl)-N′-phenylurea;
N-(1-methyl-6-phenyl-4H-[1,2,4]triazolo[4,3-a][1,4]benzodiazepin-4-yl)-N′-(phenylmethyl)urea;
N-{[4-(methyloxy)phenyl]methyl}-N′-(1-methyl-6-phenyl-4H-[1,2,4]triazolo[4,3-a][1,4]benzodiazepin-4-yl)urea;
3-bromo-N-(1-methyl-6-phenyl-4H-benzo[f][1,2,4]triazolo[4,3-a][1,4]diazepin-4-yl)benzamide;
N-(1-methyl-6-phenyl-4H-benzo[f][1,2,4]triazolo[4,3-a][1,4]diazepin-4-yl)-2-naphthamide;
phenylmethyl (1-methyl-6-phenyl-4H-[1,2,4]triazolo[4,3-a][1,4]benzodiazepin-4-yl)carbamate;
ethyl 4-({[(1-methyl-6-phenyl-4H-[1,2,4]triazolo[4,3-a][1,4]benzodiazepin-4-yl)amino]carbonyl}amino)benzoate;
1-methylethyl (1-methyl-6-phenyl-4H-[1,2,4]triazolo[4,3-a][1,4]benzodiazepin-4-yl)carbamate;
4-ethyl-N-(1-methyl-6-phenyl-4H-[1,2,4]triazolo[4,3-a][1,4]benzodiazepin-4-yl)benzamide; and
or a salt salts thereof.

22. A compound according to claim 21 selected from the group consisting of
(+)-phenylmethyl (1-methyl-6-phenyl-4H-[1,2,4]triazolo[4,3-a][1,4]benzodiazepin-4-yl)carbamate;
(+)-ethyl [6-(4-chlorophenyl)-1-methyl-4H-[1,2,4]triazolo[4,3-a][1,4]benzodiazepin-4-yl]carbamate;
(+)-ethyl 1-methyl-6-phenyl-4H-[1,2,4]triazolo[4,3-a][1,4]benzodiazepin-4-ylcarbamate;
6-(methyloxy)-N-(1-methyl-6-phenyl-4H-[1,2,4]triazolo[4,3-a][1,4]benzodiazepin-4-yl)-1H-(2E)-N-(1-methyl-6-phenyl-4H-[1,2,4]triazolo[4,3-a][1,4]benzodiazepin-4-yl)-3-phenyl-2-propenamide; and
(2E)-3-(4-fluorophenyl)-N-(1-methyl-6-phenyl-4H-[1,2,4]triazolo[4,3-a][1,4]benzodiazepin-4-yl)-2-propenamide; and
salts or salt thereof.

23. (canceled)

24. A pharmaceutical composition which comprises a compound or a salt thereof according to claim 1 and one or more pharmaceutically acceptable carriers, diluents or excipients.

25. A combination pharmaceutical product comprising a compound or a salt thereof according to claim 1 together with one or more other therapeutically active agents.

26-30. (canceled)

31. A method of treating diseases or conditions for which a bromodomain inhibitor is indicated, in a subject in need thereof which comprises administering a therapeutically effective amount of a compound or a salt thereof according to claim 1.

32. A method for treatment according to claim 31, wherein the disease or condition is a chronic autoimmune and/or inflammatory condition.

33. A method for treatment according to claim 31, wherein the disease or condition is cancer.

34. A method for treatment according to claim 31, wherein the subject is a human.

35. A method for inhibiting a bromodomain which comprises contacting the bromodomain with a compound or a salt thereof according to claim 1.
